(19) United States
(12) Patent Application Publication
(10) Pub. No.: US 2022/0317125 A1
(43) Pub. Date: Oct. 6, 2022

(54) MELANOMA BIOMARKERS

(71) Applicant: ONCIMMUNE GERMANY GMBH, Dortmund (DE)

(72) Inventors: Phil GUNNING, Nottingham (GB); Petra BUDDE, Dortmund (DE); Hans-Dieter ZUCHT, Dortmund (DE); Manuel BRÄUTIGAM, Dortmund (DE)

(21) Appl. No.: 17/596,683

(22) PCT Filed: Jun. 19, 2020
(86) PCT No.: PCT/EP2020/067245

§ 371 Date: Dec. 16, 2021

(30) Foreign Application Priority Data
Jun. 19, 2019 (GB) 1908780.8

(51) Int. Cl. G01N 33/574; G01N 33/68

(52) U.S. Cl. CPC G01N 33/5743; G01N 33/57488; G01N 33/6854

(57) ABSTRACT

The present invention relates to autoantibody biomarkers associated with melanoma. The autoantibody biomarkers can be used to detect or diagnose melanoma and can also be used to inform treatment of melanoma patients, particularly treatment with checkpoint inhibitors. The autoantibody biomarkers can be used in a variety of methods including: methods of selecting melanoma patients for treatment; methods of predicting responsiveness to treatment; methods of predicting survival responsive to treatment; and methods of predicting the risk of immune-related adverse events (irAEs) in patients treated with checkpoint inhibitors.

### FIELD OF THE INVENTION

The present invention relates to autoantibody biomarkers associated with melanoma. The autoantibody biomarkers can be used to detect or diagnose melanoma and can also be used to inform treatment of melanoma patients, particularly treatment with checkpoint inhibitors. The autoantibody biomarkers can be used in a variety of methods including: methods of selecting melanoma patients for treatment; methods of predicting responsiveness to treatment; methods of predicting survival responsive to treatment; and methods of predicting the risk of immune-related adverse events (irAEs) in patients treated with checkpoint inhibitors.

### BACKGROUND TO THE INVENTION

Melanoma, also known as malignant melanoma, is a type of skin cancer that originates from the pigment-containing melanocytes. The main factors that predispose to the development of melanoma seem to be connected with overexposure to ultraviolet sunlight and a history of sunburn.

Melanoma is the least common but the most deadly skin cancer, accounting for about 1% of all cases. According to the World Health Organization (WHO), about 132,000 melanoma skin cancers occur globally each year (http://www.who.int/uv/faq/skincancer/en/index1.html).

The survival rate for patients with melanoma depends on the thickness of the primary melanoma, whether the lymph nodes are involved, and whether the patient has developed metastasis at distant sites. The majority of patients initially present with stage I or II (localized melanoma), 8% have stage III (regional disease); and 4% have stage IV disease (distant metastases).

Surgery is the main treatment option for most cases, and usually cures early-stage melanomas.

For many decades, patients with metastatic melanoma had a very poor prognosis with a median survival time of 8-9 months. Standard of care for unresectable stage III disease or stage IV melanoma was classical therapies such as chemotherapy and radiation.

Recent progress in tumor immunology research has led to a fourth therapy option that consists of approaches to stimulate the human immune system to identify and destroy developing tumors (cancer immunotherapy or immune-oncology treatment).

An effective immune response to cancer is dependent on the capacity to detect the tumor as foreign. Many tumor cells express abnormal proteins and molecules, which in theory should be recognized by the immune system. Proteins, which are present in the tumor and elicit an immune response, are called tumor-associated antigens (TAA). The group of TAA comprises mutated proteins, overexpressed or aberrantly expressed proteins, proteins produced by oncogenic viruses, germline-expressed proteins, glycoproteins or proteins, which are produced in small quantities or are not exposed to the immune system. The immune response to TAA includes cellular processes as well as the production of antibodies against TAA that lead to the elimination of tumor cells.

However, following prolonged antigen exposure the tumor can develop immune escape mechanisms that induce functionally exhausted T effector cells. Such immune escape mechanisms include down-regulation of MHC class I molecules on tumor cells to evade antigen-presentation to T effector cells. Another immune escape mechanism of tumor cells is the upregulation of PD-1 ligand (PD-L1, also called B7-H1) on tumor cells, which inhibits the function of tumor-infiltrating T cells. Such negative regulators of immune response pathways are collectively called immune checkpoints.

The development of therapeutic antibodies that modulate immune inhibitory pathways has been a major breakthrough in the treatment of melanoma. Currently, antibodies targeting the cytotoxic T-lymphocyte-associated antigen 4 (CTLA-4) and programmed death 1 (PD-1)/PD-L1 pathway have demonstrated improved survival in patients with advanced melanoma.

Immune checkpoints are negative regulators of T-cell immune function when bound to their respective ligands CD80/86 and programmed cell-death ligand 1 and 2 (PD-L1/PD-L2).

In addition, drugs targeting other checkpoints such as lymphocyte activation gene 3 protein (LAG3), T cell immunoglobulin mucin 3 (TIM-3), and IDO (Indoleamine 2,3-dioxygenase) are in development.

Ipilimumab (Yervoy), an inhibitor of CTLA-4, is approved for the treatment of advanced or unresectable melanoma. Nivolumab (Opdivo) and pembrolizumab (Keytruda), both PD-1 inhibitors, are approved to treat patients with advanced or metastatic melanoma.

Anti-PD-L1 inhibitor avelumab (Bavencio) has received orphan drug designation by the European Medicines Agency for the treatment of gastric cancer in January 2017. The US Food and Drug Administration (FDA) approved it in March 2017 for Merkel-cell carcinoma, an aggressive type of skin cancer.

Despite the fact that checkpoint inhibitors have greatly improved the survival of advanced metastatic melanoma, non-responsiveness is also observed with only about 30% of patients appearing to benefit from ipilimumab (anti-CTLA-4) treatment (Callahan et al., 2013). Compared with ipilimumab, nivolumab and pembrolizumab (targeting PD-1) have shown increased efficacy in metastatic melanoma. Efficacy may be even further increased when using a combination of nivolumab with ipilimumab, which is also approved for metastatic melanoma and has demonstrated a 2-year overall survival rate of 63.8% (Hodi et al., 2016).

The potent ability of checkpoint inhibitors to activate the immune system can result in tissue specific inflammation characterized as immune-related adverse events (irAEs). The main side effects include diarrhea, colitis, hepatitis, skin toxicities, arthritis, diabetes, endocrinopathies such as hypophysitis and thyroid dysfunction (Spain et al., 2016). In particular, the combination therapy of nivolumab with ipilimumab led to a rate of high-grade irAEs of 55%, compared with 27% or 16% for nivolumab or ipilimumab monotherapy, respectively (Larkin et al., 2015).

Although infrequent, one of the most concerning effects of ipilimumab and combination therapies of ipilimumab, is the development of severe and even life-threatening colitis. Therefore, biomarkers are needed to predict both clinical efficacy and toxicity. Such biomarkers may guide patient selection for both monotherapy and combination therapy (Topalian et al., 2016).

There are apparent differences between the CTLA-4 and PD-1 pathways of the immune response. CTLA-4 acts more globally on the immune response by stopping potentially autoreactive T cells at the initial stage of naive T-cell activation, typically in lymph nodes. The PD-1 pathway regulates previously activated T cells at the later stages of an immune response, primarily in peripheral tissues (Buchbinder and Desai, 2016).

Substantial efforts have been undertaken to identify biomarkers for predicting which patient will respond best to immune checkpoint inhibition. Given the mechanism of action of inhibiting the PD-1 pathway, several studies have evaluated the expression of the PD-L1 ligand in the tumor as a biomarker of clinical response. However, differences regarding the predictive value of PD-L1 expression have been found. This limits the current use of PD-L1 as a biomarker for predicting clinical response. The differences in the utility of PD-L1 as a biomarker may be caused by differences in the assay type used in different studies and by variable expression of PD-L1 during therapy (Manson et al., 2016).

Since checkpoint inhibition is typically viewed as enhancing the activity of effector T cells in the tumor and tumor environment, other biomarker approaches have focused on identifying TAA recognized by T cells. However, this approach is limited to exploratory analyses and is not practical in a routine laboratory setting because it requires patient-specific MHC reagents (Gulley et al., 2014).

A largely overlooked immune cell type in the context of immunotherapies are B cells, which can exert both anti-tumor and tumor-promoting effects by providing co-stimulatory signals and inhibitory signals for T cell activation, cytokines, and antibodies (Chiaruttini et al., 2017).

Furthermore, B cells also express the immune checkpoint regulators PD-1, PD-L1, and CTLA-4 (Chiaruttini et al., 2017). Thus, administration of agents that modulate immune checkpoint molecules may also have effects on B cell activation and autoantibody production.

B cells produce anti-tumor antibodies, which can mediate antibody-dependent cellular cytotoxicity (ADCC) of tumor cells and activation of the complement cascade. It is well established that many cancer types induce an antibody response, which can be used for diagnostic purposes. Although some cancer patients show an antibody response to neo-antigens restricted to the tumor, the majority of antibodies in cancer patients are directed to self-antigens and are therefore autoantibodies (Bei et al., 2009). Breakthrough of tolerance and elevated levels of autoantibodies to self-antigens are also a prominent feature of many autoimmune diseases.

Thus, autoantibodies hold the potential to serve as biomarkers of a sustained humoral anti-tumor response/non-response and irAE in cancer patients treated with immunotherapeutic approaches.

Compared to biomarker strategies involving the identification of TAA-specific T-cells, the identification of autoantibodies can be performed using modern multiplex high-throughput screening approaches using minimal amounts of serum (Budde et al., 2016).

### SUMMARY OF INVENTION

The present application reports the identification of autoantibody biomarkers associated with melanoma. The autoantibody biomarkers described herein have been linked to treatment of melanoma patients, particularly treatment of melanoma patients with checkpoint inhibitors. The autoantibody biomarkers can be used to inform treatment decisions and/or to predict different aspects of patient response to treatment with checkpoint inhibitors.

In a first aspect, the present invention provides methods of selecting melanoma patients for treatment with one or more checkpoint inhibitors. In accordance with this first aspect, provided herein is a method of selecting a melanoma patient for treatment with one or more checkpoint inhibitors, the method comprising:

(a) determining in a sample obtained from the patient the level(s) of autoantibodies specifically binding to one or more of the antigens selected from the following:

ACTB, AMPH, AQP4, BAG6, BICD2, BIRC5, C15orf48, C17orf85, CALR, CCNB1, CENPH, CENPV, CEP131, CTAG1B, CTSW, EIF3E, EOMES, FGFR1, FLNA, FRS2, GNAI2, GPHN, GRP, GSK3A, HES1, IGF2BP2, IL23A, IL36RN, KRT19, MAZ, MIF, MLLT6, MUM1, NCOA1, NOVA2, NRIP1, PAPOLG, PPP1R2, PTPRR, RALY, SDCBP, SIVA1, SNRNP70, SNRPA, SNRPD1, SPA17, SSB, SUM02, TEX264, TMEM98, TRAF3IP3, XRCC5 and XRCC6; and

(b) comparing the level(s) of autoantibodies determined in (a) with a predetermined cut-off value for autoantibodies specifically binding to the same one or more antigens,

wherein if the level of autoantibodies determined in the patient sample is higher than the predetermined cut-off value, the patient is selected for treatment with the checkpoint inhibitor(s).

Further provided is a method of selecting a melanoma patient for treatment with one or more checkpoint inhibitors, the method comprising:

(a) determining in a sample obtained from the patient the level(s) of autoantibodies specifically binding to one or more of the antigens selected from the following:

ABCB8, AKT2, AMPH, AP1S1, AP2B1, ATG4D, ATP13A2, BTBD2, BTRC, CAP2, CASP10, CASP8, CFB, CREB3L1, CTSW, EGFR, EIF4E2, ELMO2, EOMES, ERBB3, FADD, FGA, FN1, FOXO1, FRS2, GABARAPL2, HSPA1B, HSPB1, IL23A, IL3, IL4R, KDM4A, KLKB1, KRT7, L1CAM, LAMB2, LAMC1, LEPR, LGALS3BP, MAGEB4, MAGED2, MAPT, MITF, MUC12, MUM1, OGT, PCDH1, PDCD6IP, PECAM1, PIAS3, PLIN2, PPL, PPP1R12A, PRKCI, RAPGEF3, RELT, RPLP0, RPLP2, SIGIRR, SIPA1L1, SPA17, SPTB, SPTBN1, SUFU, TEX264, TMEM98, TOLLIP, TONSL, TP53, TPM2, TRIP4, UBAP1, UBE2Z, UBTF, XRCC5 and XRCC6; and

(b) comparing the level(s) of autoantibodies determined in (a) with a predetermined cut-off value for autoantibodies specifically binding to the same one or more antigens,

wherein if the level of autoantibodies determined in the patient sample is not higher than the predetermined cut-off value, the patient is selected for treatment with the checkpoint inhibitor(s).

Further provided is a method of selecting a melanoma patient for treatment with one or more checkpoint inhibitors, the method comprising:

(a) determining in a sample obtained from a patient the level(s) of autoantibodies specifically binding to one or more of the antigens selected from the following:

ARRB1, BCL7B, CCDC51, CEACAM5, CSNK2A1, DFFA, DHFR, FGFR1, GNG12, GRAMD4, GRK6, HDAC1, LAMC1, MSH2, MIF, MMP3, RPS6KA1, S100A8, S100A14, SHC1 and USB1; and

(b) comparing the level(s) of autoantibodies determined in (a) with a predetermined cut-off value for autoantibodies specifically binding to the same one or more antigens,

wherein if the level of autoantibodies determined in the patient sample is lower than the pre-determined cut-off value, the patient is selected for treatment with the checkpoint inhibitor(s).

Further provided is a method of selecting a melanoma patient for treatment with one or more checkpoint inhibitors, the method comprising:

(a) determining in a sample obtained from a patient the level(s) of autoantibodies specifically binding to one or more of the antigens selected from the following:

CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1; and

(b) comparing the level(s) of autoantibodies determined in (a) with a predetermined cut-off value for autoantibodies specifically binding to the same one or more antigens,

wherein if the level of autoantibodies determined in the patient sample is not lower than the pre-determined cut-off value, the patient is selected for treatment with the checkpoint inhibitor(s).

In a further aspect, the present invention provides a method of treating melanoma in a subject, the method comprising administering to the subject one or more checkpoint inhibitors, wherein the subject is selected for treatment in accordance with the methods of the first aspect of the invention.

In a further aspect, the present invention provides methods of predicting melanoma patients' responsiveness to treatment with a checkpoint inhibitor. Provided herein is a method of predicting a melanoma patient's responsiveness to treatment with a checkpoint inhibitor, the method comprising:

(a) determining in a sample obtained from a patient the level(s) of autoantibodies specifically binding to one or more of the antigens selected from the following:

ACTB, AQP4, BIRC5, C15orf48, C17orf85, CALR, CCNB1, CENPH, CENPV, CEP131, CTAG1B, EOMES, FGA, FLNA, FRS2, GNAI2, GPHN, GSK3A, HES1, IGF2BP2, IL17A, IL36RN, MAZ, MLLT6, NOVA2, NRIP1, PAPOLG, PPP1R2, PTPRR, RALY, SIVA1, SNRNP70, SNRPA, SNRPD1, SSB, TEX264, TRAF3IP3, XRCC5 and XRCC6, and

(b) comparing the level(s) of autoantibodies determined in (a) with a predetermined cut-off value for autoantibodies specifically binding to the same one or more antigens,

wherein if the level of autoantibodies determined in the patient sample is higher than the predetermined cut-off value, improved responsiveness is predicted.

Further provided is a method of predicting a melanoma patient's responsiveness to treatment with a checkpoint inhibitor, the method comprising:

(a) determining in a sample obtained from a patient the level(s) of autoantibodies specifically binding to one or more of the antigens selected from the following:

GRK6, MIF, FGFR1 GRAMD4, GNG12, CCDC51, USB1, RPS6KA1, BCL7B, S100A14, MMP3, SHC1, CSNK2A1, DFFA, S100A8, HDAC1, MSH2, CEACAM5, DHFR, LAMC1 and ARRB1; and

(b) comparing the level of autoantibodies determined in (a) with a predetermined cut-off value for autoantibodies specifically binding to the same one or more antigens,

wherein if the level of autoantibodies determined in the patient sample is lower than the predetermined cut-off value, improved responsiveness is predicted.

In a further aspect, the present invention provides methods of predicting survival in melanoma patients responsive to treatment with checkpoint inhibitors. Provided herein is a method of predicting survival in a melanoma patient responsive to treatment with a checkpoint inhibitor, the method comprising:

(a) determining in a sample obtained from a patient the level(s) of autoantibodies specifically binding to one or more of the antigens selected from the following:

ACTB, AQP4, BIRC5, C15orf48, C17orf85, CALR, CCNB1, CENPH, CENPV, CEP131, CTAG1B, EOMES, FGA, FLNA, FRS2, GNAI2, GPHN, GSK3A, HES1, IGF2BP2, IL17A, IL36RN, MAZ, MLLT6, NOVA2, NRIP1, PAPOLG, PPP1R2, PTPRR, RALY, SIVA1, SNRNP70, SNRPA, SNRPD1, SSB, TEX264, TRAF3IP3, XRCC5 and XRCC6; and

(b) comparing the level(s) of autoantibodies determined in (a) with a predetermined cut-off value for autoantibodies specifically binding to the same one or more antigens,

wherein if the level of autoantibodies determined in the patient sample is higher than the predetermined cut-off value, improved survival is predicted.

Further provided is a method of predicting survival in a melanoma patient responsive to treatment with a checkpoint inhibitor, the method comprising:

(a) determining in a sample obtained from a patient the level(s) of autoantibodies specifically binding to one or more of the antigens selected from the following:

GRK6, MIF, FGFR1 GRAMD4, GNG12, CCDC51, USB1, GRAMD4, RPS6KA1, BCL7B, S100A14, MMP3, SHC1, CSNK2A1, DFFA, S100A8, HDAC1, MSH2, CEACAM5, DHFR, LAMC1 and ARRB1; and

(b) comparing the level of autoantibodies determined in (a) with a predetermined cut-off value for autoantibodies specifically binding to the same one or more antigens,

wherein if the level of autoantibodies determined in the patient sample is lower than the predetermined cut-off value, improved survival is predicted.

In a further aspect, the present invention provides methods of predicting the risk of immune-related adverse events (irAEs) in melanoma patients treated with one or more checkpoint inhibitors. Provided herein is a method of predicting the risk of immune-related adverse events (irAEs) in a melanoma patient treated with one or more checkpoint inhibitors, the method comprising:

(a) determining in a sample obtained from a patient the level(s) of autoantibodies specifically binding to one or more of the antigens selected from the following:

TEX264, CREB3L1, HSPA1B, SPTB, MUC12, ERBB3, CASP10, FOXO1, FRS2, PPP1R12A, CAP2, EOMES, CREB3L1, PLIN2, SIPA1L1, ABCB8, MAPT, XRCC5, XRCC6, UBAP1, TRIP4, EIF4E2, FADD, OGT, HSPB1, CAP2, ATP13A2, SIGIRR, HSPA1B, SPTB, PDCD6IP, RAPGEF3, PECAM1, PPL, TONSL, ELMO2, LAMB2, BTRC, SUFU, LGALS3BP, KLKB1, EGFR, TOLLIP, MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17; and

(b) comparing the level of autoantibodies determined in (a) with a predetermined cut-off value for autoantibodies specifically binding to the same one or more antigens,

wherein if the level of autoantibodies determined in the patient sample is higher than the predetermined cut-off value, the patient is determined to be at higher risk of irAEs.

Further provided is a method of predicting the risk of immune-related adverse events (irAEs) in a melanoma patient treated with one or more checkpoint inhibitors, the method comprising:

(a) determining in a sample obtained from a patient the level(s) of autoantibodies specifically binding to one or more of the antigens selected from the following:

SUM02, GRP, SDCBP, AMPH, GPHN, BAG6, BICD2, TMEM98, MUM1, CTSW, NCOA1, MIF, SPA17, FGFR1, KRT19; and

(ii) comparing the level of autoantibodies determined in (a) with a predetermined cut-off value for autoantibodies specifically binding to the same one or more antigens,

wherein if the level of autoantibodies determined in the patient sample is higher than the predetermined cut-off value, the patient is determined to be at lower risk of irAEs.

Further provided is a method of predicting the risk of immune-related adverse events (irAEs) in a melanoma patient treated with one or more checkpoint inhibitors, the method comprising:

(a) determining in a sample obtained from a patient the level(s) of autoantibodies specifically binding to one or more of the antigens selected from the following:

CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1; and

(b) comparing the level of autoantibodies determined in (a) with a predetermined cut-off value for autoantibodies specifically binding to the same one or more antigens, wherein if the level of autoantibodies determined in the patient sample is lower than the predetermined cut-off value, the patient is determined to be at higher risk of irAEs.

The autoantibody biomarkers described herein can also be used to detect or diagnose melanoma.

In a further aspect, the present invention provides a method of detecting melanoma in a mammalian subject by detecting an autoantibody in a sample obtained from the mammalian subject,

wherein the autoantibody specifically binds to an antigen selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, and NOVA2,

wherein the presence of autoantibodies at a level above a pre-determined cut-off value is indicative of melanoma;

and/or

wherein the autoantibody specifically binds to an antigen selected from: SNRPA, NRIP1, UBAP1, TEX264, PLIN2, LAMC1, CENPH, USB1, ABCB8, C15orf48/NMES1, and MAGED1, wherein the presence of autoantibodies at a level below a pre-determined cut-off value is indicative of melanoma.

In a still further aspect, the present invention provides a method of diagnosing melanoma in a mammalian subject by detecting an autoantibody in a sample obtained from the mammalian subject,

wherein the autoantibody specifically binds to an antigen selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, and NOVA2,

wherein the subject is diagnosed as having melanoma if the presence of autoantibodies is at a level above a pre-determined cut-off value; and/or

wherein the autoantibody specifically binds to an antigen selected from: SNRPA, NRIP1, UBAP1, TEX264, PLIN2, LAMC1, CENPH, USB1, ABCB8, C15orf48/NMES1, and MAGED1, wherein the subject is diagnosed as having melanoma if the presence of autoantibodies is at a level below a pre-determined cut-off value.

The present invention also provides kits suitable for performing the methods of the invention.

Further provided are uses of the antigens described herein in the methods of the preceding aspects.

### BRIEF DESCRIPTION OF DRAWINGS

FIG. 1 illustrates a design of the cancer screen. KEGG Pathway Analysis (Kyoto Encyclopedia of Genes and Genomes) of human proteins and antigens included in the cancer autoantibody screen. Proteins were selected to represent the following three categories: natural and autoimmune antigens, tumor-associated antigens, immune-related pathways and dysregulated pathways in autoimmune diseases, cancer signaling pathways, and proteins or genes overexpressed in different cancer types. The individual categories are listed on the x-axis, with the number of proteins per category is indicated at the y-axis.

FIG. 2 illustrates the number of analyzed patients and serum samples per immune-oncology treatment, or therapy. Pre-treatment samples were collected before initiation of therapy, and post-treatment samples were collected at approximately 3 and 6 months following treatment.

FIG. 3 illustrates the best response according to RECIST 1.1 for 193 melanoma patients in percentage per immune-oncology therapy. PD: progressive disease; SD: stable disease; PR: partial response; and CR: complete response.

FIG. 4 illustrates immune-related adverse events (irAEs) for 193 melanoma patients in percentage per immune-oncology therapy. The graph shows the percentage of all irAEs per treatment as well as detailed information of specific irAEs.

FIG. 5 illustrates Box-and-Whisker plots and ROC curves of three autoantibodies in melanoma patients and healthy controls (HC). Box-and-Whisker plots and ROC (Receiver Operating Characteristics) curves of IgG autoantibody reactivities against CREB3L, CXCL5, and NME1 in serum samples of melanoma patients and healthy controls. Numbers at the y-axis indicate the log 2 Luminex Median Fluorescence Intensity values (MFI).

FIG. 6 illustrates Box-and-Whisker plots of baseline autoantibodies predicting DCR or PD to all forms of checkpoint inhibitor treatment. Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI). Pre-treatment samples of patients treated with different checkpoint inhibitors (FIG. 2) are jointly analyzed.

FIG. 7 illustrates Box-and-Whisker plots and ROC curves of two baseline autoantibodies predicting irAEs in melanoma patients. Box-and-Whisker plots and ROC curves show a comparison of pre-treatment IgG autoantibody levels of patients who develop or do not develop irAEs following treatment with checkpoint inhibitors. Pre-treatment samples of patients treated with different checkpoint inhibitors (FIG. 2) are jointly analyzed.

FIG. 8 illustrates Box-and-Whisker Plots of baseline autoantibodies predicting DCR or PD to ipilimumab. Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI). Pre-treatment samples of patients treated with anti-CTLA-4 blocker ipilimumab are analyzed.

FIG. 9 illustrates Box-and-Whisker plots of baseline autoantibodies predicting irAE in ipilimumab-treated patients. Box-and-Whisker plots show a comparison of pre-treatment IgG autoantibody levels of patients who develop or do not develop irAEs following treatment with checkpoint inhibitors. Pre-treatment (T0 samples) of patients treated with anti-CTLA-4 blocker ipilimumab are analyzed.

FIG. 10 illustrates Box-and-Whisker plots of baseline autoantibodies predicting DCR or PD to pembrolizumab. Box-and-Whisker plots show a comparison of pre-treatment IgG autoantibody levels of patients with progressive disease (PD) and those achieving disease control rate (DCR). DCR is defined as CR, PR, or SD. Numbers at the y-axis indicate the log 2 Luminex Median Fluorescence Intensity values (MFI). Baseline (T0) samples of patients treated with anti-PD-1/PD-L1 pathway blocker pembrolizumab are analyzed.

FIG. 11 illustrates Box-and-Whisker Plots of baseline autoantibodies predicting irAE in pembrolizumab-treated patients. Box-and-Whisker plots show a comparison of pre-treatment IgG autoantibody levels of patients who develop or do not develop irAEs following treatment with checkpoint inhibitors. Pre-treatment (T0 samples) of patients treated with anti-CTLA-4 blocker pembrolizumab are analyzed.

FIG. 12 illustrates study samples and data analysis workflow. For data mining patients were regrouped into the following modeling cohorts: “all treatments”=complete patient cohort; “ipi-ever”=patients treated with ipi-mono, ipi/nivo or pembro with prior ipi; “ipi-mono”=ipi-mono cohort; “pembro-never-ipi”=pembro-treated patients without prior ipi.

FIG. 13 illustrates summary statistics for 47 autoantibodies predicting irAE and colitis. Autoantibodies predicting an adverse event (colitis are irAE) are highlighted in black, whereas those predicting a reduced risk are shown in white.

FIG. 14 illustrates Kaplan Meier curves with confidence intervals of baseline autoantibodies and their targets predicting colitis. Serum autoantibody levels were dichotomized and Kaplan Meier curves for patients with high and low autoantibody levels plotted. X-axis: Time (days), and Y-axis: Event probability.

FIG. 15 illustrates Kaplan Meier curves with confidence intervals of pre-treatment autoantibodies and their targets predicting irAE. Serum autoantibody levels were dichotomized and Kaplan Meier curves for patients with high and low autoantibody levels plotted. X-axis: Time (days), and Y-axis: Event probability.

FIG. 16 illustrates optimized marker combinations for prediction of colitis (A) and irAE (B). Filled circles: Positive predictive autoantibodies, grey circles: negative predictive autoantibodies

### DETAILED DESCRIPTION

A. Definitions

Unless defined otherwise, technical and scientific terms used herein have the same meaning as commonly understood by one of ordinary skill in the art to which this invention belongs.

The terms “a”, “an”, and “the” do not denote a limitation of quantity, but rather denote the presence of “at least one” of the referenced item. In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The term “about” or “approximately” means within a statistically meaningful range of a value.

Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

As used herein, “autoantibody” means an antibody produced by the immune system of a subject that is directed to and specifically binds to an “autoantigen”, “self-antigen” or an “antigenic epitope” thereof. The terms “specifically bind” and/or “specifically recognize” as used herein, refer to the higher affinity of a binding molecule for a target molecule compared to the binding molecule's affinity for non-target molecules. A binding molecule that specifically binds a target molecule does not substantially recognize or bind non-target molecules, e.g., an antibody “specifically binds” and/or “specifically recognizes” another molecule, meaning that this interaction is dependent on the presence of the binding specificity of the molecule structure, e.g., an antigenic epitope.

As used herein, the term “autoantibody biomarker” refers to an autoantibody, the levels of which are associated with a particular phenotype, response or outcome. Autoantibody biomarkers in accordance with the present invention are associated with melanoma and/or the response of melanoma patients to treatment with checkpoint inhibitors. As described herein, the levels of autoantibody biomarkers can be detected in samples obtained from subjects/patients and the levels can be compared with pre-determined cut-off values. This assessment of autoantibody biomarkers can be used to detect/diagnose melanoma as well as inform decisions relating to treatment of melanoma patients with checkpoint inhibitors.

As used herein, the terms “diagnose” or “diagnosis” or “diagnosing” refer to determining the nature or the identity of a condition or disease or disorder, e.g., melanoma, detecting and/or classifying the melanoma in a subject. A diagnosis may be accompanied by a determination as to the severity of the melanoma.

As used herein, the term “sample” refers to a sample obtained from a mammalian subject or a patient for evaluation in vitro. The sample can be any sample that is expected to contain antibodies and/or immune cells. The sample can be taken from blood, e.g., serum, peripheral blood, peripheral blood mononuclear cells (PBMC), whole blood or whole blood pre-treated with an anticoagulant such as heparin, ethylenediamine tetraacetic acid, plasma or serum. A sample may be pre-treated prior to use, such as by preparing plasma from blood, diluting viscous liquids, or the like. Methods of treating a sample may also involve separation, filtration, distillation, concentration, inactivation of interfering components, and/or the addition of reagents.

As used herein, the terms “treat,” “treatment,” “treating,” or “amelioration” refer to therapeutic treatments, wherein the object is to reverse, alleviate, ameliorate, inhibit, slow down or stop the progression or severity of melanoma, an associated condition and/or a symptom thereof. The term “treating” includes reducing or alleviating at least one adverse effect or symptom of melanoma. Treatment is generally “effective” if one or more symptoms or clinical markers are reduced. Alternatively, or in addition, treatment is “effective” if the progression of a disease is reduced or halted.

As used herein, the term “disease control rate” or “DCR” can be used as a measure of clinical response to treatment in a cohort of patients, for example clinical response to a checkpoint inhibitor. The DCR is the percentage of patients achieving complete response (CR), or partial response (PR) or stable disease (SD).

As used herein, the term “checkpoint inhibitor” refers to an agent that inhibits an immune checkpoint protein or pathway so as to stimulate or promote the body's anti-tumour response.

Preferred checkpoint inhibitors in accordance with the present invention include CTLA-4 inhibitors and inhibitors of the PD-L1/PD-1 pathway.

CTLA-4 (cytotoxic T-lymphocyte-associated protein 4), also known as CD152 (cluster of differentiation 152), is a protein receptor that functions as an immune checkpoint and downregulates immune responses. CTLA-4 is constitutively expressed in regulatory T cells but only upregulated in conventional T cells after activation—a phenomenon which is particularly notable in cancers. CTLA-4 acts as an “off” switch when bound to CD80 or CD86 on the surface of antigen-presenting cells. CTLA-4 has been identified as an interesting target for the development of checkpoint inhibitor therapies and ipilimumab, a monoclonal antibody inhibitor of CTLA-4, was approved for treating melanoma by the FDA and EMA in 2011.

PD-1 and its ligands, particularly PD-L1, have been relatively well-characterised as immune checkpoint regulators, and dysregulation of the PD-1-PD-L1 signalling pathway in the cancer microenvironment has been identified as an important means by which tumours suppress the immune response. The receptor PD-1 is typically expressed on a variety of immune cells including monocytes, T cells, B cells, dendritic cells and tumour-infiltrating lymphocytes, and the ligand PD-L1 has been found to be upregulated on a number of different types of tumour cell (see Ohaegbulam et al. (2015) Trends Mol Med. 21(1):24-33, incorporated herein by reference).

The interaction between PD-L1 on tumour cells and PD-1 on immune cells, particularly T cells, creates an immunosuppressive tumour microenvironment via effects at the level of CD8+ cytotoxic T cells and also via the generation of Treg cells (see Alsaab et al. (2017) Front Pharmacol. Aug 23(8):561, incorporated herein by reference). Many agents capable of inhibiting the activity of PD-1, PD-L1 or the PD1-PD-L1 signalling axis have been developed as reported for example, in Alsaab et al. ibid (incorporated by reference). PD-1 inhibitors include but not limited to: nivolumab; pembrolizumab; pidilizumab, REGN2810; AMP-224; MEDI0680; and PDR001. PD-L1 inhibitors include but are not limited to: atezolizumab; and avelumab.

The term “immune-related adverse event” or “irAE” as used herein refers to the adverse events caused by the use of checkpoint inhibitors as a result of the stimulation of the immune system.

Immune-related adverse events are typically associated with tissue inflammation and can include but are not limited to colitis, diarrhea, hepatitis, skin toxicities, arthritis, diabetes, endocrinopathies such as hypophysitis, and thyroid dysfunction.

B. Methods Using Melanoma Autoantibody Biomarkers

Methods of Selecting Melanoma Patients for Treatment with Checkpoint Inhibitors and Methods of Treating Melanoma Patients Selected for Treatment

In a first aspect, the present invention provides methods of selecting melanoma patients for treatment with one or more checkpoint inhibitors. The methods comprise a step of analysing a sample obtained from a melanoma patient to determine the levels of autoantibodies specifically binding to one or more target antigens. The sample is typically removed from the body such that the analysis of the sample is carried out in vitro.

The patient may be a patient previously diagnosed with melanoma or suspected of having melanoma. The patient may have been diagnosed or may be diagnosed in accordance with any method for the diagnosis of melanoma. The patient may have received prior treatment for melanoma or may be newly-diagnosed having received no prior treatment. The patient may have failed on previous treatment or suffered a relapse such that a new treatment regime is required. The patient may have melanoma at any stage of disease progression, for example stage I, stage II, stage III or stage IV disease. In preferred embodiments, the patient has metastatic melanoma.

In certain embodiments, the patient is a subject at increased risk of developing melanoma, e.g. due to: family history; carrying alleles or a genotype associated with melanoma; a history of excessive sun exposure; or the existence of moles and/or lesions associated with later development of melanoma.

The sample obtained for in vitro analysis in accordance with the methods described herein may be any sample expected to contain autoantibodies and/or immune cells. The sample may be taken from blood, e.g., serum, peripheral blood, peripheral blood mononuclear cells (PBMC), whole blood or whole blood pre-treated with an anticoagulant such as heparin, ethylenediamine tetraacetic acid, plasma or serum. The sample is preferably serum. The sample may be pre-treated prior to testing, such as by preparing plasma from blood, diluting viscous liquids, or the like. Methods of treating the sample prior to testing may also involve separation, filtration, distillation, concentration, inactivation of interfering components, and/or the addition of reagents.

The sample may also be stored prior to testing. In certain embodiments, the sample may be any one of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears or wound drainage fluid.

In accordance with the methods of the invention, the sample obtained from the patient is assessed for autoantibodies, also referred to herein as “autoantibody biomarkers”. The autoantibody biomarkers analysed in accordance with this first aspect of the invention can be used to select melanoma patients for treatment with checkpoint inhibitors on the basis that the autoantibodies have been linked to one or more of: clinical response; survival; and the development of immune-related adverse events (irAEs) in patients treated with checkpoint inhibitors, particularly the checkpoint inhibitors ipilimumab, nivolumab, pembrolizumab and combinations thereof.

In certain embodiments, the patient sample is tested for autoantibody biomarkers that bind to one or more of the antigens selected from: ACTB, AMPH, AQP4, BAG6, BICD2, BIRC5, C15orf48, C17orf85, CALR, CCNB1, CENPH, CENPV, CEP131, CTAG1B, CTSW, EIF3E, EOMES, FGFR1, FLNA, FRS2, GNAI2, GPHN, GRP, GSK3A, HES1, IGF2BP2, IL23A, IL36RN, KRT19, MAZ, MIF, MLLT6, MUM1, NCOA1, NOVA2, NRIP1, PAPOLG, PPP1R2, PTPRR, RALY, SDCBP, SIVA1, SNRNP70, SNRPA, SNRPD1, SPA17, SSB, SUM02, TEX264, TMEM98, TRAF3IP3, XRCC5 and XRCC6. Autoantibody biomarkers that bind to one or more of the antigens listed in this group may be considered positive predictive biomarkers for patient selection in this aspect of the invention. The levels of these autoantibodies have been reported as increased in patients exhibiting improved clinical response and/or improved survival and/or reduced risk of irAEs responsive to treatment with checkpoint inhibitors. For embodiments wherein one or more of the positive predictive biomarkers listed above is analysed, a higher level of autoantibodies in the patient sample as compared with a pre-determined cut-off value identifies the patient as a patient suitable for treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13 14, 15, 16, 17, 18, 19 or 20 antigens from the above list. Panel embodiments as described herein are contemplated for use in all aspects of the invention.

In certain embodiments, the autoantibodies bind to one or more antigens selected from: SIVA1, IGF2BP2, AQP4, C15orf48, CTAG1B and PAPOLG. In certain embodiments, the autoantibodies bind to 1, 2, 3, 4, 5 or 6 antigens selected from: SIVA1, IGF2BP2, AQP4, C15orf48, CTAG1B and PAPOLG.

In certain embodiments, the autoantibodies bind to one or more antigens selected from: FRS2, BIRC5, EIF3E, CENPH and PAPOLG. In certain embodiments, the autoantibodies bind to 1, 2, 3, 4 or 5 antigens selected from: FRS2, BIRC5, EIF3E, CENPH and PAPOLG.

In certain embodiments, the autoantibody biomarkers bind to one or more antigens selected from: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2 and IL36RN. In certain embodiments, the autoantibody biomarkers bind to 1, 2, 3, 4, 5, 6, 7, 8, 9, 10, 11, 12, 13, 14, 15, 16, 17, 18, 19, 20, 21, 22, 23, 24, 25, 26 27, 28 or 29 antigens selected from: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2 and IL36RN.

In certain embodiments, the autoantibody biomarkers bind to one or more antigens selected from: SUM02, GRP, SDCBP, AMPH, IL23A, GPHN, BAG6, BICD2, TMEM98, MUM1, CTSW, NCOA1, MIF, SPA17, FGFR1 and KRT19. In certain embodiments, the autoantibody biomarkers bind to 1, 2, 3, 4, 5, 6, 7, 8, 9, 10, 11, 12, 13, 14, 15 or 16 antigens selected from: SUM02, GRP, SDCBP, AMPH, IL23A, GPHN, BAG6, BICD2, TMEM98, MUM1, CTSW, NCOA1, MIF, SPA17, FGFR1 and KRT19.

In certain embodiments, the patient sample is tested for autoantibody biomarkers that bind to one or more of the antigens selected from: ABCB8, AKT2, AMPH, AP1S1, AP2B1, ATG4D, ATP13A2, BTBD2, BTRC, CAP2, CASP10, CASP8, CFB, CREB3L1, CTSW, EGFR, EIF4E2, ELMO2, EOMES, ERBB3, FADD, FGA, FN1, FOXO1, FRS2, GABARAPL2, HSPA1B, HSPB1, IL23A, IL3, IL4R, KDM4A, KLKB1, KRT7, L1CAM, LAMB2, LAMC1, LEPR, LGALS3BP, MAGEB4, MAGED2, MAPT, MITF, MUC12, MUM1, OGT, PCDH1, PDCD6IP, PECAM1, PIAS3, PLIN2, PPL, PPP1R12A, PRKCI, RAPGEF3, RELT, RPLP0, RPLP2, SIGIRR, SIPA1L1, SPA17, SPTB, SPTBN1, SUFU, TEX264, TMEM98, TOLLIP, TONSL, TP53, TPM2, TRIP4, UBAP1, UBE2Z, UBTF, XRCC5 and XRCC6. Autoantibody biomarkers that bind to one or more of the antigens listed in this group have been reported as increased in patients at increased risk of irAEs responsive to treatment with checkpoint inhibitors. It follows that if the levels of autoantibodies in the patient sample are not higher or are lower than the pre-determined cut-off value, the patient is selected for treatment with the checkpoint inhibitor(s) on the basis that the patient is not at increased risk of suffering an irAEs responsive to treatment.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13 14, 15, 16, 17, 18, 19 or 20 antigens from the above list.

In certain embodiments, the autoantibodies bind to one or more antigens selected from: TEX264, CREB3L1, HSPA1B, SPTB, MUC12, ERBB3, CASP10, FOXO1, FRS2, PPP1R12A and CAP2. In certain embodiments, the autoantibodies bind to 1, 2, 3, 4, 5, 6, 7, 8, 9, 10 or 11 antigens selected from: TEX264, CREB3L1, HSPA1B, SPTB, MUC12, ERBB3, CASP10, FOXO1, FRS2, PPP1R12A and CAP2.

In certain embodiments, the autoantibodies bind to one or more antigens selected from: EOMES, CREB3L1, FRS2, PLIN2, SIPA1L1, ABCB8, MAPT, XRCC5, XRCC6, UBAP1, TRIP4 and EIF4E2. In certain embodiments, the autoantibodies bind to 1, 2, 3, 4, 5, 6, 7, 8, 9, 10, 11 or 12 antigens selected from: EOMES, CREB3L1, FRS2, PLIN2, SIPA1L1, ABCB8, MAPT, XRCC5, XRCC6, UBAP1, TRIP4 and EIF4E2.

In certain embodiments, the autoantibodies bind to one or more antigens selected from: FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP. In certain embodiments, the autoantibodies bind to 1, 2, 3, 4, 5, 6, 7, 8, 9, 10, 11, 12, 13, 14, 15, 16, 17, 18, 19, 20, 21, 22 or 23 antigens selected from: FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

In certain embodiments, the autoantibodies bind to one or more antigens selected from:

MAGED2, PIAS3, MITF, AP2B1, PRKCI, AKT2, BTBD2, UBE2Z, L1CAM, GABARAPL2, LAMC1, RPLP2, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4, CTSW, ATG4D, TPM2 and SPA17. In certain embodiments, the autoantibodies bind to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 antigens selected from: MAGED2, PIAS3, MITF, AP2B1, PRKCI, AKT2, BTBD2, UBE2Z, L1CAM, GABARAPL2, LAMC1, RPLP2, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4, CTSW, ATG4D, TPM2 and SPA17.

In certain embodiments, the patient sample is tested for autoantibody biomarkers that bind to one or more of the antigens selected from: ARRB1, BCL7B, CCDC51, CEACAM5, CSNK2A1, DFFA, DHFR, FGFR1, GNG12, GRAMD4, GRK6, HDAC1, LAMC1, MSH2, MIF, MMP3, RPS6KA1, S100A8, S100A14, SHC1 and USB1. Autoantibody biomarkers that bind to one or more of the antigens listed in this group may be considered negative predictive biomarkers for patient selection in this aspect of the invention. The levels of these autoantibodies have been reported as decreased in patients exhibiting improved clinical response and/or improved survival responsive to treatment with checkpoint inhibitors. For embodiments wherein one or more of the negative predictive biomarkers listed above is analysed, a lower level of autoantibodies in the patient sample as compared with a pre-determined cut-off value identifies the patient as a patient suitable for treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13 14, 15, 16, 17, 18, 19 or 20 antigens from the above list.

In certain embodiments, the autoantibodies bind to one or more antigens selected from: GRK6 and GRAMD4. In certain embodiments, the autoantibodies bind to 1 or 2 antigens selected from: GRK6 and GRAMD4.

In certain embodiments, the autoantibodies bind to one or more antigens selected from: GNG12, CCDC51, USB1, GRAMD4, RPS6KA1 and BCL7B. In certain embodiments, the autoantibodies bind to 1, 2, 3, 4, 5 or 6 antigens selected from: GNG12, CCDC51, USB1, GRAMD4, RPS6KA1 and BCL7B.

In certain embodiments, the autoantibodies bind to one or more antigens selected from: S100A14, MMP3, SHC1, CSNK2A1, DFFA, S100A8, HDAC1, MSH2, CEACAM5, DHFR and ARRB1. In certain embodiments, the autoantibodies bind to 1, 2, 3, 4, 5, 6, 7, 8, 9, 10, 11 or 12 antigens selected from: S100A14, MMP3, SHC1, CSNK2A1, DFFA, S100A8, HDAC1, MSH2, CEACAM5, DHFR and ARRB1.

In certain embodiments, the patient sample is tested for autoantibody biomarkers that bind to one or more of the antigens selected from: CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1.

Autoantibody biomarkers that bind to one or more of the antigens listed in this group have been reported as decreased in patients at increased risk of irAEs responsive to treatment with checkpoint inhibitors. It follows that if the levels of autoantibodies in the patient sample are not lower or are higher than the pre-determined cut-off value, the patient is selected for treatment with the checkpoint inhibitor(s) on the basis that the patient is not at increased risk of suffering an irAEs responsive to treatment.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more antigens from the above list.

In certain embodiments, the patient sample is tested for autoantibodies binding to a panel of 2, 3, 4, 5, 6, 7, 8, 9, 10, 11, 12, 13 14 or 15 antigens from the above list.

In certain embodiments, the autoantibodies bind to one or more antigens selected from: HSPA2, SMAD9, HIST2H2AA3 and S100A8. In certain embodiments, the autoantibodies bind to 1, 2, 3 or 4 antigens selected from: HSPA2, SMAD9, HIST2H2AA3 and S100A8.

In certain embodiments, the autoantibodies bind to one or more antigens selected from: POLR3B, ELMO2, RFWD2, SQSTM1, HSPD1 and IL17A. In certain embodiments, the autoantibodies bind to 1, 2, 3, 4, 5 or 6 antigens selected from: POLR3B, ELMO2, RFWD2, SQSTM1, HSPD1 and IL17A.

In certain embodiments, the autoantibodies bind to one or more antigens selected from: CXXC1, LARP1, EGLN2, RPRM, WHSC1L1 and S100A8. In certain embodiments, the autoantibodies bind to 1, 2, 3, 4, 5 or 6 antigens selected from: CXXC1, LARP1, EGLN2, RPRM, WHSC1L1 and S100A8.

In certain embodiments, the methods in accordance with the first aspect of the invention may involve the analysis of autoantibody levels for autoantibody biomarkers binding to any combination of antigens described in the context of this first aspect of the invention. For example, the methods may involve the analysis of a combination of positive predictive biomarkers and negative predictive biomarkers as described herein. Alternatively or in addition, the methods may involve the analysis of a combination of biomarkers associated with increased and/or decreased risk or irAEs. Any combination of autoantibody biomarkers may be analysed in accordance with the first aspect of the invention.

The methods require the level of each autoantibody biomarker in the patient sample to be determined or measured. This measurement can be made using any suitable immunoassay technique for the detection of autoantibodies. The general features of immunoassays, for example ELISA, radio-immunoassays and the like, are well known to those skilled in the art (see Immunoassay, E. Diamandis and T. Christopoulus, Academic Press, Inc., San Diego, Calif., 1996, the contents of which are incorporated herein by reference). Immunoassays for the detection of autoantibodies having a particular immunological specificity generally require the use of a reagent (antigen) that exhibits specific immunological reactivity with a relevant autoantibody.

Depending on the format of the assay, this antigen may be immobilised on a solid support. A test sample is brought into contact with the antigen and if autoantibodies of the required immunological specificity are present in the sample they will immunologically react with the antigen to form antigen/autoantibody complexes which may then be detected or quantitatively measured. The immunoassay used to detect autoantibodies according to the invention may be based on standard techniques known in the art.

The detection of autoantibody may be carried out in any suitable format which enables contact between the sample suspected of containing the autoantibody (the “test sample”) and the antigen. Conveniently, contact between the patient sample and the antigen may take place in separate reaction chambers such as the wells of a microtitre plate, allowing different antigens or different amounts of antigen to be assayed in parallel, if required. For immunoassays in which varying amounts of the antigen are used, these can be coated onto the wells of the microtitre plate by preparing serial dilutions from a stock of antigen across the wells of the microtitre plate.

The stock of antigen may be of known or unknown concentration. Aliquots of the test sample may then be added to the wells of the plate, with the volume and dilution of the test sample kept constant in each well. The absolute amounts of antigen added to the wells of the microtitre plate may vary depending on such factors as the nature of the target autoantibody, the nature of the test sample, dilution of the test sample etc. as will be appreciated by those skilled in the art.

Generally, the amounts of antigen and the dilution of the test sample will be selected so as to produce a range of signal strengths which fall within the acceptable detection range of the read-out chosen for detection of antigen/autoantibody binding in the method.

In some embodiments, a patient sample, preferably serum, is contacted with a sample of the antigen immobilised at a discrete location or reaction site on a solid support. Solid supports include but are not limited to filters, membranes, beads (for example magnetic or fluorophore-labelled beads), small plates, silicon wafers, glass, metal, plastic, chips, mass spectrometry targets or matrices. In some embodiments, the solid support is a bead. In some embodiments, the bead is a microsphere.

For embodiments wherein autoantibodies that specifically bind to multiple antigens are being detected, the antigens may be coupled to multiple different solid supports and then arranged onto an array. The array may be in the form of a “protein array”, wherein a protein array refers to the systematic arrangement of melanoma antigens on a solid support, wherein the melanoma antigens are proteins or peptides or parts thereof. Protein arrays or “microarrays” may be used to perform multiple assays for autoantibodies of different specificity on a single sample in parallel.

This can be done using arrays comprising multiple antigens or sets of antigens.

In certain embodiments, and depending on the precise nature of the assay in which it will be used, the antigen may comprise a naturally occurring protein, or fragment thereof, linked to one or more further molecules which impart some desirable characteristic not naturally present in the protein. For example, the protein or fragment may be conjugated to a revealing label, such as for example a fluorescent label, coloured label, luminescent label, radiolabel or heavy metal such as colloidal gold. In other embodiments the protein or fragment may be expressed as a recombinantly produced fusion protein. By way of example, fusion proteins may include a tag peptide at the N- or C-terminus to assist in purification of the recombinantly expressed antigen.

The level of any given autoantibody biomarker in the patient sample may be determined by measuring the degree of binding between the autoantibody present in the sample and the antigen. Binding between autoantibody and antigen can be visualized, for example, by means of fluorescence labelling, biotinylation, radio-isotope labelling or colloid gold or latex particle labelling. Suitable techniques are known to those skilled in the art and may be employed in the methods of the invention. Bound autoantibodies may be detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemiluminescent substrates. A read-out can be determined, for example by means of a microarray laser scanner, a CCD camera or visually.

In a most preferred embodiment the immunoassay used to detect autoantibodies in accordance with the invention is an ELISA. ELISAs are generally well known in the art. In a typical indirect ELISA an antigen having specificity for the autoantibodies under test is immobilised on a solid surface (e.g. the wells of a standard microtiter assay plate, or the surface of a microbead or a microarray) and a sample to be tested for the presence of autoantibodies is brought into contact with the immobilised antigen. Any autoantibodies of the desired specificity present in the sample will bind to the immobilised antigen. The bound antigen/autoantibody complexes may then be detected using any suitable method. In one preferred embodiment a labelled secondary anti-human immunoglobulin antibody, which specifically recognises an epitope common to one or more classes of human immunoglobulins, is used to detect the antigen/autoantibody complexes. Typically the secondary antibody will be anti-IgG or anti-IgM. The secondary antibody is usually labelled with a detectable marker, typically an enzyme marker such as, for example, peroxidase or alkaline phosphatase, allowing quantitative detection by the addition of a substrate for the enzyme which generates a detectable product, for example a coloured, chemiluminescent or fluorescent product. Other types of detectable labels known in the art may be used with equivalent effect.

In a further step of the methods of the invention, the level or levels of autoantibody biomarkers determined in the patient sample are compared with pre-determined cut-off values for autoantibodies specifically binding to the same antigens. The pre-determined cut-off value may be different for different autoantibodies. The pre-determined cut-off value will have been calculated or may be calculated based on the analysis of a control cohort of melanoma patients.

In particular, the pre-determined cut-off for any given autoantibody biomarker will typically be the average level of autoantibodies determined in a control cohort of melanoma patients.

As reported herein, the autoantibody biomarkers used in the methods of the present invention can be found in the serum of melanoma patients (see Examples 7 and 8 and Tables 1 and 2).

The autoantibodies measured in accordance with the methods serve as useful biomarkers because their baseline levels i.e. their levels prior to the start of checkpoint inhibitor treatment, were found to be increased or decreased in those patients exhibiting responses such as a clinical response to treatment, improved survival and/or increased/decreased irAEs, as compared with the overall melanoma patient population assessed.

The “control cohort of melanoma patients” from which the pre-determined cut-off value is calculated for any given antigen may be any reasonably-sized cohort of melanoma patients, for example at least 50 patients, at least 100 patients, at least 200 patients, at least 500 patients.

The pre-determined cut-off value against which the autoantibodies of the melanoma patient sample are compared in accordance with the methods of the invention may be pre-determined based upon a particular control cohort of melanoma patients matched to the patient under test.

For example, the pre-determined cut-off value of autoantibodies may be determined on the basis of a cohort of melanoma patients matched for any one of the following criteria with the patient under test: type of melanoma; disease stage; age; gender; use of pre-existing melanoma treatment.

Once the level of autoantibodies in the patient sample has been compared with the pre-determined cut-off value for autoantibodies specifically binding to the same target antigen, an assessment is made as to whether the level of autoantibodies in the patient sample is higher, lower, not higher or not lower than the predetermined cut-off value. As reported herein, this comparison allows a decision to be made as to whether or not the patient is selected for treatment.

For embodiments wherein the patient sample is tested for autoantibody biomarkers that bind to one or more antigens selected from: ACTB, AMPH, AQP4, BAG6, BICD2, BIRC5, C15orf48, C17orf85, CALR, CCNB1, CENPH, CENPV, CEP131, CTAG1B, CTSW, EIF3E, EOMES, FGFR1, FLNA, FRS2, GNAI2, GPHN, GRP, GSK3A, HES1, IGF2BP2, IL23A, IL36RN, KRT19, MAZ, MIF, MLLT6, MUM1, NCOA1, NOVA2, NRIP1, PAPOLG, PPP1R2, PTPRR, RALY, SDCBP, SIVA1, SNRNP70, SNRPA, SNRPD1, SPA17, SSB, SUM02, TEX264, TMEM98, TRAF3IP3, XRCC5 and XRCC6, a higher level of autoantibodies in the patient sample as compared with the pre-determined cut-off value may identify the patient as a patient suitable for treatment with a checkpoint inhibitor or a combination of checkpoint inhibitors. A level of autoantibodies that is higher than the pre-determined cut-off value indicates that the patient is likely to exhibit improved responsiveness, improved survival and/or a reduced risk of irAEs responsive to treatment with a checkpoint inhibitor.

For embodiments wherein the patient sample is tested for autoantibody biomarkers that bind to one or more antigens selected from: ABCB8, AKT2, AMPH, AP1S1, AP2B1, ATG4D, ATP13A2, BTBD2, BTRC, CAP2, CASP10, CASP8, CFB, CREB3L1, CTSW, EGFR, EIF4E2, ELMO2, EOMES, ERBB3, FADD, FGA, FN1, FOXO1, FRS2, GABARAPL2, HSPA1B, HSPB1, IL23A, IL3, IL4R, KDM4A, KLKB1, KRT7, L1CAM, LAMB2, LAMC1, LEPR, LGALS3BP, MAGEB4, MAGED2, MAPT, MITF, MUC12, MUM1, OGT, PCDH1, PDCD6IP, PECAM1, PIAS3, PLIN2, PPL, PPP1R12A, PRKCI, RAPGEF3, RELT, RPLP0, RPLP2, SIGIRR, SIPA1L1, SPA17, SPTB, SPTBN1, SUFU, TEX264, TMEM98, TOLLIP, TONSL, TP53, TPM2, TRIP4, UBAP1, UBE2Z, UBTF, XRCC5 and XRCC6, a level of autoantibodies in the patient sample that is not higher or is lower than the pre-determined cut-off value may identify the patient as a patient suitable for treatment with a checkpoint inhibitor or a combination of checkpoint inhibitors. A level of autoantibodies that is not higher or is lower than the pre-determined cut-off value indicates that the patient is not at increased risk of irAEs.

For embodiments wherein the patient sample is tested for autoantibody biomarkers that bind to one or more antigens selected from: ARRB1, BCL7B, CCDC51, CEACAM5, CSNK2A1, DFFA, DHFR, FGFR1, GNG12, GRAMD4, GRK6, HDAC1, LAMC1, MSH2, MIF, MMP3, RPS6KA1, S100A8, S100A14, SHC1 and USB1, a lower level of autoantibodies in the patient sample as compared with the pre-determined cut-off value may identify the patient as a patient suitable for treatment with a checkpoint inhibitor or a combination of checkpoint inhibitors. A level of autoantibodies that is lower than the pre-determined cut-off value indicates that the patient is likely to exhibit improved responsiveness and/or improved survival responsive to treatment with a checkpoint inhibitor.

For embodiments wherein the patient sample is tested for autoantibody biomarkers that bind to one or more antigens selected from: CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1, a level of autoantibodies in the patient sample that is not lower or is higher than the pre-determined cut-off value may identify the patient as a patient suitable for treatment with a checkpoint inhibitor or a combination of checkpoint inhibitors. A level of autoantibodies that is not lower or is higher than the pre-determined cut-off value indicates that the patient is not at increased risk of irAEs.

For embodiments wherein the autoantibody is assessed as “higher” or “lower” than the pre-determined cut-off value, a threshold may be applied. For example, a threshold may be applied such that the autoantibodies in the patient sample must be at least 1.5 fold higher or lower, at least 2 fold higher or lower, at least 2.5 fold higher or lower than the pre-determined cut-off value for the patient to be selected for treatment. A threshold may be applied such that the autoantibodies in the patient sample must be at least 10%, at least 20%, at least 50% higher or lower than the pre-determined cut-off value for the patient to be selected for treatment.

For embodiments wherein the method involves determining the levels of autoantibodies specifically binding to multiple antigens, as described above, the patient may be selected for treatment with a checkpoint inhibitor if the autoantibody levels for at least one of the antigens are higher or lower than the pre-determined cut-off value for autoantibodies specifically binding to that antigen. For embodiments wherein the method involves determining the levels of autoantibodies specifically binding to multiple antigens, as described above, the patient may be selected for treatment with a checkpoint inhibitor if the autoantibody levels for at least two, at least three, at least four, at least five of the antigens are higher or lower than the pre-determined cut-off values for autoantibodies specifically binding to the corresponding antigens. In some embodiments wherein the method involves determining the levels of autoantibodies binding to multiple antigens, the patient may be selected for treatment if the levels of autoantibodies specifically binding to each antigen tested are higher or lower than the pre-determined cut-off values for autoantibodies specifically binding to the corresponding antigens.

The methods described herein may be used to select melanoma patients for treatment with one or more checkpoint inhibitors wherein the checkpoint inhibitors are selected from any such inhibitors known to those skilled in the art, particularly checkpoint inhibitors known for use in the treatment of melanoma patients. In preferred embodiments, the methods are used to select melanoma patients for treatment with a checkpoint inhibitor selected from a CTLA-4 inhibitor, a PD-1 inhibitor and a PD-L1 inhibitor. The methods may be used to select patients for treatment with a combination therapy comprising a CTLA-4 inhibitor, a PD-1 inhibitor and/or a PD-L1 inhibitor. In particular embodiments, the methods are used to select patients for treatment with a combination therapy comprising a CTLA-4 inhibitor and a PD-1 inhibitor. The CTLA-4 inhibitor, PD-1 inhibitor and/or PD-L1 inhibitor may be selected from any known inhibitors of these checkpoint proteins and pathways. The inhibitors are preferably antibodies or antigen-binding fragments thereof that bind to CTLA-4, PD-1 and/or PD-L1. In preferred embodiments, the patients are selected for treatment with the anti-CTLA-4 antibody ipilimumab. In preferred embodiments, the patients are selected for treatment with the anti-PD-1 antibody nivolumab. In preferred embodiments, the patients are selected for treatment with the anti-PD-1 antibody pembrolizumab. In preferred embodiments, the patients are selected for treatment with a combination of ipilimumab and nivolumab.

The methods described herein may comprise an additional step of administering the one or more checkpoint inhibitors to the patient. The one or more checkpoint inhibitors may be administered to the melanoma patient via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration.

In a further aspect, the present invention also provides methods of treating melanoma patients with one or more checkpoint inhibitors wherein the patients have been selected for treatment by methods in accordance with any embodiments of the first aspect of the invention. Also provided herein are checkpoint inhibitors for use in treating melanoma in patients in need thereof wherein the patients are selected for treatment by methods in accordance with any embodiments of the first aspect of the invention.

Methods of Predicting Response and/or Survival

In further aspects, the present invention provides methods of predicting a melanoma patient's responsiveness to treatment with a checkpoint inhibitor and methods of predicting survival in a melanoma patient responsive to treatment with a checkpoint inhibitor. The steps of the methods of these further aspects are similar to the steps described above for the methods in accordance with the first aspect of the invention. As such, all embodiments pertaining to the first aspect of the invention are equally applicable to these further aspects of the invention. In particular, these embodiments pertain to patients selected for testing, the nature of the patient sample, and the methods by which the autoantibody levels may be determined in the patient sample.

The methods of these further aspects comprise a step of analysing a sample obtained from a melanoma patient to determine the levels of autoantibodies specifically binding to one or more target antigens. The autoantibodies analysed in accordance with these further aspects of the invention serve as biomarkers of clinical response and/or patient survival, as reported herein.

In certain embodiments, the patient sample is tested for autoantibody biomarkers that bind to one or more of the antigens selected from: ACTB, AQP4, BIRC5, C15orf48, C17orf85, CALR, CCNB1, CENPH, CENPV, CEP131, CTAG1B, EOMES, FGA, FLNA, FRS2, GNAI2, GPHN, GSK3A, HES1, IGF2BP2, IL17A, IL36RN, MAZ, MLLT6, NOVA2, NRIP1, PAPOLG, PPP1R2, PTPRR, RALY, SIVA1, SNRNP70, SNRPA, SNRPD1, SSB, TEX264, TRAF3IP3, XRCC5 and XRCC6. Autoantibody biomarkers that bind to one or more of the antigens listed in this group may be considered positive predictive biomarkers for clinical response and/or survival. The levels of these autoantibodies have been reported as increased in patients exhibiting an improved clinical response and/or improved survival responsive to treatment with checkpoint inhibitors. For embodiments wherein one or more of the positive predictive biomarkers listed above is analysed, a higher level of autoantibodies in the patient sample as compared with a pre-determined cut-off value is predictive of improved responsiveness and/or improved survival following treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13 14, 15, 16, 17, 18, 19 or 20 antigens from the above list.

In certain embodiments, the autoantibodies bind to one or more antigens selected from: SIVA1, IGF2BP2, AQP4, C15orf48, CTAG1B and PAPOLG. In certain embodiments, the autoantibodies bind to 1, 2, 3, 4, 5 or 6 antigens selected from: SIVA1, IGF2BP2, AQP4, C15orf48, CTAG1B and PAPOLG.

In certain embodiments, the autoantibodies bind to one or more antigens selected from: FRS2, GHPN, BIRC5, EIF3E, CENPH and PAPOLG. In certain embodiments, the autoantibodies bind to 1, 2, 3, 4, 5 or 6 antigens selected from: FRS2, GHPN, BIRC5, EIF3E, CENPH and PAPOLG. For these embodiments, the methods are preferably for predicting response to treatment with ipilimumab.

In certain embodiments, the autoantibodies bind to one or more antigens selected from: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2, IL36RN, FGA and GHPN. In certain embodiments, the autoantibodies bind to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 antigens selected from: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2, IL36RN, FGA and GHPN. For these embodiments, the methods are preferably for predicting response to treatment with pembrolizumab.

In certain embodiments of these aspects of the invention, the patient sample is tested for autoantibody biomarkers that bind to one or more of the antigens selected from: GRK6, MIF, FGFR1 GRAMD4, GNG12, CCDC51, USB1, RPS6KA1, BCL7B, S100A14, MMP3, SHC1, CSNK2A1, DFFA, S100A8, HDAC1, MSH2, CEACAM5, DHFR, LAMC1 and ARRB1.

Autoantibody biomarkers that bind to one or more of the antigens listed in this group may be considered negative predictive biomarkers for clinical response and/or survival. The levels of these autoantibodies have been reported as decreased in patients exhibiting an improved clinical response and/or improved survival responsive to treatment with checkpoint inhibitors. For embodiments wherein one or more of the negative predictive biomarkers listed above is analysed, a lower level of autoantibodies in the patient sample as compared with a pre-determined cut-off value is predictive of improved survival following treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13 14, 15, 16, 17, 18, 19 or 20 antigens from the above list.

In certain embodiments, the autoantibodies bind to one or more antigens selected from: GRK6 and GRAMD4. In certain embodiments, the autoantibodies bind to 1 or 2 antigens selected from: GRK6 and GRAMD4.

In certain embodiments, the autoantibodies bind to one or more antigens selected from: GNG12, CCDC51, USB1, GRAMD4, RPS6KA1, BCL7B. In certain embodiments, the autoantibodies bind to 1, 2, 3, 4, 5, 6, 7 or antigens selected from: GNG12, CCDC51, USB1, GRAMD4, RPS6KA1, BCL7B. For these embodiments, the methods are preferably for predicting response to treatment with ipilimumab.

In certain embodiments, the autoantibodies bind to one or more antigens selected from: S100A14, MMP3, SHC1, CSNK2A1, DFFA, S100A8, HDAC1, MSH2, CEACAM5, DHFR, LAMC1 and ARRB1. In certain embodiments, the autoantibodies bind to 1, 2, 3, 4, 5, 6, 7, 8, 9, 10, 11 or 12 antigens selected from: S100A14, MMP3, SHC1, CSNK2A1, DFFA, S100A8, HDAC1, MSH2, CEACAM5, DHFR, LAMC1 and ARRB1. For these embodiments, the methods are preferably for predicting response to treatment with pembrolizumab.

In a further step of the methods, the level or levels of autoantibody biomarkers determined in the patient sample are compared with pre-determined cut-off values for autoantibodies specifically binding to the same antigens. The pre-determined cut-off value for any given autoantibody biomarker is calculated as described above in relation to the first aspect of the invention. Once the level of autoantibodies in the patient sample has been compared with the pre-determined cut-off value for autoantibodies specifically binding to the same target antigen, an assessment is made as to whether the level of autoantibodies in the patient sample is higher or lower than the predetermined cut-off value. As reported herein, this comparison allows a prediction to be made regarding the patient's likelihood of improved responsiveness and/or improved survival following treatment with one or more checkpoint inhibitors.

For embodiments wherein the autoantibody level is assessed as “higher” or “lower” than the pre-determined cut-off value, a threshold may be applied. For example, a threshold may be applied such that the autoantibodies in the patient sample must be at least 1.5 fold higher or lower, at least 2 fold higher or lower, at least 2.5 fold higher or lower than the pre-determined cut-off value for the patient to be predicted as having improved responsiveness or improved survival. A threshold may be applied such that the autoantibodies in the patient sample must be at least 10%, at least 20%, at least 50% higher or lower than the pre-determined cut-off value for the patient to be predicted as having improved responsiveness or improved survival.

For embodiments wherein the method involves determining the levels of autoantibodies specifically binding to multiple antigens, as described above, the patient may be predicted as having improved responsiveness or improved survival if the autoantibody levels for at least one of the antigens are higher or lower than the pre-determined cut-off value for autoantibodies specifically binding to that antigen. For embodiments wherein the method involves determining the levels of autoantibodies specifically binding to multiple antigens, as described above, the patient may be predicted as having improved responsiveness or improved survival if the autoantibody levels for at least two, at least three, at least four, at least five of the antigens are higher or lower than the pre-determined cut-off values for autoantibodies specifically binding to the corresponding antigens. In some embodiments wherein the method involves determining the levels of autoantibodies binding to multiple antigens, the patient may be predicted as having improved responsiveness or improved survival if the levels of autoantibodies specifically binding to each antigen tested are higher or lower than the pre-determined cut-off values for autoantibodies specifically binding to the corresponding antigens.

The methods described herein for predicting responsiveness to treatment with a checkpoint inhibitor are intended for the prediction of clinical response in any given melanoma patient. As used herein, the term “improved responsiveness” should be taken to mean an improved clinical response as compared with the average response seen in a control cohort of melanoma patients treated with the same checkpoint inhibitor or combination of checkpoint inhibitors. As described herein, clinical response to treatment may be assessed by measuring a patient's complete response (CR), a patient's partial response (PR) or the existence of stable disease (SD). The average response for melanoma patients may be determined or known from prior clinical trials or case control studies.

The methods described herein for predicting survival in melanoma patients treated with checkpoint inhibitors may be used to predict various aspects of survival, for example, overall survival (OS), 5-year survival, 2-year survival and/or progression-free survival (PFS). As used herein, the term “improved survival” should be taken to mean improved survival as compared with the average survival seen in a control cohort of melanoma patients treated with the same checkpoint inhibitor or combination of checkpoint inhibitors. The average survival may be determined or known from prior clinical trials or case control studies.

The methods described herein may be used to predict clinical response or survival responsive to treatment with any checkpoint inhibitors, particularly checkpoint inhibitors known for use in the treatment of melanoma patients. In preferred embodiments, the methods are used to predict clinical response or survival responsive to treatment with a checkpoint inhibitor selected from a CTLA-4 inhibitor, a PD-1 inhibitor and a PD-L1 inhibitor. The methods may be used to predict clinical response or survival responsive to treatment with a combination therapy comprising a CTLA-4 inhibitor, a PD-1 inhibitor and/or a PD-L1 inhibitor. In particular embodiments, the methods are used to predict clinical response or survival responsive to treatment with a combination therapy comprising a CTLA-4 inhibitor and a PD-1 inhibitor. The CTLA-4 inhibitor, PD-1 inhibitor and/or PD-L1 inhibitor may be selected from any known inhibitors of these checkpoint proteins and pathways. The inhibitors are preferably antibodies or antigen-binding fragments thereof that specifically bind to CTLA-4, PD-1 and/or PD-L1. In preferred embodiments, the anti-CTLA-4 antibody is ipilimumab. In preferred embodiments, the anti-PD-1 antibody is nivolumab or pembrolizumab. In preferred embodiments, the methods are used to predict clinical response or survival responsive to treatment with a combination therapy comprising ipilimumab and nivolumab.

Methods of Predicting the Risk of Immune-Related Adverse Events (irAEs)

In a further aspect, the present invention provides methods of predicting the risk of immune-related adverse events (irAEs) in a melanoma patient treated with one or more checkpoint inhibitors. The steps of the methods of this further aspect are similar to the steps described above for the methods in accordance with the first aspect of the invention. As such, all embodiments pertaining to the first aspect of the invention are equally applicable to this further aspect of the invention. In particular, these embodiments pertain to patients selected for testing, the nature of the patient sample, and the methods by which the autoantibody levels may be measured in the patient sample.

The methods of this further aspect comprise a step of analysing a sample obtained from a melanoma patient to determine the levels of autoantibodies specifically binding to one or more target antigens. In this aspect, the autoantibodies serve as biomarkers predictive of the risk of irAEs.

In certain embodiments, the patient sample is tested for autoantibody biomarkers that bind to one or more of the antigens selected from: TEX264, CREB3L1, HSPA1B, SPTB, MUC12, ERBB3, CASP10, FOXO1, FRS2, PPP1R12A, CAP2, EOMES, CREB3L1, PLIN2, SIPA1L1, ABCB8, MAPT, XRCC5, XRCC6, UBAP1, TRIP4, EIF4E2, FADD, OGT, HSPB1, CAP2, ATP13A2, SIGIRR, HSPA1B, SPTB, PDCD6IP, RAPGEF3, PECAM1, PPL, TONSL, ELMO2, LAMB2, BTRC, SUFU, LGALS3BP, KLKB1, EGFR, TOLLIP, MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17. Autoantibody biomarkers that bind to one or more of the antigens listed in this group may be considered positive predictive biomarkers for an increased risk of irAEs. The levels of these autoantibodies have been reported as increased in patients experiencing irAEs responsive to treatment with checkpoint inhibitors. For embodiments wherein one or more of these positive predictive biomarkers is analysed, a higher level of autoantibodies in the patient sample as compared with a pre-determined cut-off value identifies the patient as a patient at increased risk of experiencing irAEs following treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens from the above list. In certain embodiments, the patient sample is tested for autoantibodies binding to a panel of 2, 3, 4, 5, 6, 7, 8, 9, 10, 11, 12, 13, 14, 15, 16, 17, 18, 19 or 20 antigens from the above list.

In certain embodiments, the autoantibodies bind to one or more antigens selected from: TEX264, CREB3L1, HSPA1B, SPTB, MUC12, ERBB3, CASP10, FOXO1, FRS2, PPP1R12A and CAP2. In certain embodiments, the autoantibodies bind to 1, 2, 3, 4, 5 6, 7, 8, 9, 10 or 11 antigens selected from: TEX264, CREB3L1, HSPA1B, SPTB, MUC12, ERBB3, CASP10, FOXO1, FRS2, PPP1R12A and CAP2.

In certain embodiments, the autoantibodies bind to one or more antigens selected from: EOMES, CREB3L1, FRS2, PLIN2, SIPA1L1, ABCB8, MAPT, XRCC5, XRCC6, UBAP1, TRIP4 and EIF4E2. In certain embodiments, the autoantibodies bind to 1, 2, 3, 4, 5, 6, 7, 8, 9, 10, 11 or 12 antigens selected from: EOMES, CREB3L1, FRS2, PLIN2, SIPA1L1, ABCB8, MAPT, XRCC5, XRCC6, UBAP1, TRIP4 and EIF4E2.

In certain embodiments, the autoantibodies bind to one or more antigens selected from: FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP. In certain embodiments, the autoantibodies bind to 1, 2, 3, 4, 5, 6, 7, 8, 9, 10, 11, 12, 13, 14, 15, 16, 17, 18, 19, 20, 21, 22 or 23 antigens selected from: FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

In certain embodiments, the autoantibodies bind to one or more antigens selected from: MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4, CTSW, ATG4D, TPM2 and SPA17. In certain embodiments, the autoantibodies bind to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 antigens selected from: MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4, CTSW, ATG4D, TPM2 and SPA17.

In certain embodiments, the autoantibodies bind to one or more antigens selected from: IL4R, L1CAM, MITF, PIAS3, AP1S1, ATG4D and RPLP2. In certain embodiments, the autoantibodies bind to 1, 2, 3, 4, 5, 6 or 7 antigens selected from: IL4R, L1CAM, MITF, PIAS3, AP1S1, ATG4D and RPLP2.

In certain embodiments, the autoantibodies bind to one or more antigens selected from: RELT, CASP8, UBE2Z, IL4R, LAMC1, L1CAM and MITF. In certain embodiments, the autoantibodies bind to 1, 2, 3, 4, 5, 6 or 7 antigens selected from: RELT, CASP8, UBE2Z, IL4R, LAMC1, L1CAM and MITF.

In certain embodiments, the autoantibodies bind to one or more antigens selected from: PIAS3, RPLP2, ATG4D, KRT7, TPM2, GABARAPL2 and MAGEB4. In certain embodiments, the autoantibodies bind to 1, 2, 3, 4, 5, 6 or 7 antigens selected from: PIAS3, RPLP2, ATG4D, KRT7, TPM2, GABARAPL2 and MAGEB4.

In certain embodiments, the autoantibodies bind to one or more antigens selected from: autoantibodies. In certain embodiments, the autoantibodies bind to 1, 2, 3, 4 or 5 antigens selected from: MAGED2, PIAS3, MITF, AP2B1 and PRKC1.

In preferred embodiments, the autoantibodies bind to MAGED2 and/or KRT7.

In certain embodiments, the autoantibodies bind to one or more antigens selected from: UBE2Z, L1CAM, GABARAPL2, CFB, IL3, RELT, FGA, and IL4R. In certain embodiments, the autoantibodies bind to 1, 2, 3, 4, 5, 6, 7 or 8 antigens selected from: UBE2Z, L1CAM, GABARAPL2, CFB, IL3, RELT, FGA, and IL4R. For these embodiments, the methods are preferably for predicting irAEs responsive to treatment with ipilimumab.

In certain embodiments, the autoantibodies bind to one or more antigens selected from: PIAS3, MITF, PRKCI, AP2B1, PDCH1, SPTBN1, and UBTF. In certain embodiments, the autoantibodies bind to 1, 2, 3, 4, 5, 6 or 7 antigens selected from: PIAS3, MITF, PRKCI, AP2B1, PDCH1, SPTBN1, and UBTF. For these embodiments, the methods are preferably for predicting irAEs responsive to treatment with the combination of ipilimumab and nivolumab.

In preferred embodiments, the methods described herein are for predicting the risk of colitis.

For embodiments wherein the irAE is colitis, the autoantibody biomarkers may bind to one or more antigens selected from MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4 and CTSW. In certain embodiments, the autoantibodies bind to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 antigens selected from: MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4 and CTSW.

In certain embodiments, the patient sample is tested for autoantibody biomarkers that bind to one or more of the antigens selected from: SUM02, GRP, SDCBP, AMPH, GPHN, BAG6, BICD2, TMEM98, MUM1, CTSW, NCOA1, MIF, SPA17, FGFR1 and KRT19. Autoantibody biomarkers that bind to one or more of the antigens listed in this group may be considered positive predictive biomarkers for a decreased risk of irAEs. The levels of these autoantibodies have been reported as increased in patients at reduced risk of irAEs responsive to treatment with checkpoint inhibitors. For embodiments wherein one or more of these positive predictive biomarkers is analysed, a higher level of autoantibodies in the patient sample as compared with a pre-determined cut-off value identifies the patient as a patient at lower risk of experiencing irAEs following treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more or fourteen or more antigens from the above list. In certain embodiments, the patient sample is tested for autoantibodies binding to a panel of 2, 3, 4, 5, 6, 7, 8, 9, 10, 11, 12, 13, 14 or 15 antigens from the above list.

In certain embodiments, the autoantibodies bind to one or more antigens selected from: NCOA1, MIF, SDCB4, MUM1, FGFR1 and KRT19. In certain embodiments, the autoantibodies bind to 1, 2, 3, 4, 5 or 6 antigens selected from: NCOA1, MIF, SDCB4, MUM1, FGFR1 and KRT19.

In certain embodiments, the autoantibodies bind to one or more antigens selected from: MIF, NCOA1, FGFR1 and SDCBP. In certain embodiments, the autoantibodies bind to 1, 2, 3 or 4 antigens selected from: MIF, NCOA1, FGFR1 and SDCBP.

In certain embodiments, the autoantibodies bind to one or more antigens selected from: SUMO2, GRP and MIF. In certain embodiments, the autoantibodies bind to 1, 2 or 3 antigens selected from: SUMO2, GRP and MIF.

In preferred embodiments, the methods described herein are for predicting the risk of colitis.

For embodiments wherein the irAE is colitis, the autoantibody biomarkers may bind to one or more antigens selected from SUM02, GRP, SDCBP, AMPH, GPHN, BAG6, BICD2, TMEM98 and MUM1. In certain embodiments, the autoantibody biomarkers bind to 1, 2, 3, 4, 5, 6, 7, 8 or 9 antigens selected from: SUM02, GRP, SDCBP, AMPH, GPHN, BAG6, BICD2, TMEM98 and MUM1.

In certain embodiments, the patient sample is tested for autoantibody biomarkers that bind to one or more of the antigens selected from: CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1.

Autoantibody biomarkers that bind to one or more of the antigens listed in this group may be considered negative predictive biomarkers for increased risk of irAEs. The levels of these autoantibodies have been reported as decreased in patients at increased risk of irAEs responsive to treatment with checkpoint inhibitors. For embodiments wherein one or more of these negative predictive biomarkers is analysed, a lower level of autoantibodies in the patient sample as compared with a pre-determined cut-off value identifies the patient as a patient at higher risk of experiencing an irAE following treatment with a checkpoint inhibitor or a combination of checkpoint inhibitors.

The sensitivity of the methods may be increased by testing for multiple autoantibodies i.e. autoantibodies that bind to multiple different antigens. In this regard, the patient sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more or fourteen or more antigens from the above list. In certain embodiments, the patient sample is tested for autoantibodies binding to a panel of 2, 3, 4, 5, 6, 7, 8, 9, 10, 11, 12, 13, 14 or 15 antigens from the above list.

In certain embodiments, the autoantibodies bind to one or more antigens selected from: HSPA2, SMAD9, HIST2H2AA3 and S100A8. In certain embodiments, the autoantibodies bind to 1, 2, 3 or 4 antigens selected from: HSPA2, SMAD9, HIST2H2AA3, S100A8.

In certain embodiments, the autoantibodies bind to one or more antigens selected from: POLR3B, ELMO2, RFWD2, SQSTM1, HSPD1 and IL17A. In certain embodiments, the autoantibodies bind to 1, 2, 3, 4, 5 or 6 antigens selected from: POLR3B, ELMO2, RFWD2, SQSTM1, HSPD1 and IL17A. For these embodiments, the methods are preferably for predicting irAEs responsive to treatment with ipilimumab.

In certain embodiments, the autoantibodies bind to one or more antigens selected from: CXXC1, LARP1, EGLN2, RPRM, WHSC1L1 and S100A8. In certain embodiments, the autoantibodies bind to 1, 2, 3, 4, 5 or 6 antigens selected from: CXXC1, LARP1, EGLN2, RPRM, WHSC1L1 and S100A8. For these embodiments, the methods are preferably for predicting irAEs responsive to treatment with pembrolizumab.

In a further step of the methods, the level or levels of autoantibody biomarkers determined in the patient sample are compared with pre-determined cut-off values for autoantibodies specifically binding to the same antigens. The pre-determined cut-off value for any given autoantibody biomarker is calculated as described above in relation to the first aspect of the invention. Once the level of autoantibodies in the patient sample has been compared with the pre-determined cut-off value for autoantibodies specifically binding to the same target antigen, an assessment is made as to whether the level of autoantibodies in the patient sample is higher or lower than the predetermined cut-off value. As reported herein, this comparison allows a prediction to be made regarding the patient's likelihood of experiencing irAEs following treatment with one or more checkpoint inhibitors.

For embodiments wherein the autoantibody level is assessed as “higher” or “lower” than the pre-determined cut-off value, a threshold may be applied. For example, a threshold may be applied such that the autoantibodies in the patient sample must be at least 1.5 fold higher or lower, at least 2 fold higher or lower, at least 2.5 fold higher or lower than the pre-determined cut-off value for the patient to be predicted as at increased or decreased risk of irAEs. A threshold may be applied such that the autoantibodies in the patient sample must be at least 10%, at least 20%, at least 50% higher or lower than the pre-determined cut-off value for the patient to be predicted as at increased or decreased risk of irAEs.

For embodiments wherein the method involves determining the levels of autoantibodies specifically binding to multiple antigens, as described above, the patient may be considered at increased or decreased risk of irAEs if the autoantibody levels for at least one of the antigens are higher or lower than the pre-determined cut-off value for autoantibodies specifically binding to that antigen. For embodiments wherein the method involves determining the levels of autoantibodies specifically binding to multiple antigens, as described above, the patient may be considered at increased or decreased risk of irAEs if the autoantibody levels for at least two, at least three, at least four, at least five of the antigens are higher or lower than the pre-determined cut-off values for autoantibodies specifically binding to the corresponding antigens. In some embodiments wherein the method involves determining the levels of autoantibodies binding to multiple antigens, the patient may be considered at increased or decreased risk of irAEs if the levels of autoantibodies specifically binding to each antigen tested are higher or lower than the pre-determined cut-off values for autoantibodies specifically binding to the corresponding antigens.

The methods described herein may be used to predict a melanoma patient's risk of irAEs responsive to treatment with any checkpoint inhibitors, particularly checkpoint inhibitors known for use in the treatment of melanoma patients. In preferred embodiments, the methods are used to predict the risk of irAEs responsive to treatment with a checkpoint inhibitor selected from a CTLA-4 inhibitor, a PD-1 inhibitor and a PD-L1 inhibitor. The methods may be used to predict the risk of irAEs with a combination therapy comprising a CTLA-4 inhibitor, a PD-1 inhibitor and/or a PD-L1 inhibitor. In particular embodiments, the methods are used to predict the risk of irAEs responsive to treatment with a combination therapy comprising a CTLA-4 inhibitor and a PD-1 inhibitor. The CTLA-4 inhibitor, PD-1 inhibitor and/or PD-L1 inhibitor may be selected from any known inhibitors of these checkpoint proteins and pathways. The inhibitors are preferably antibodies or antigen-binding fragments thereof that specifically bind to CTLA-4, PD-1 and/or PD-L1. In preferred embodiments, the anti-CTLA-4 antibody is ipilimumab. In preferred embodiments, the anti-PD-1 antibody is nivolumab or pembrolizumab. In preferred embodiments, the methods are used to predict the risk of irAEs responsive to treatment with a combination therapy comprising ipilimumab and nivolumab.

Methods of Detecting and Diagnosing Melanoma

In further aspects, the present invention relates to methods of detecting melanoma and methods of diagnosing melanoma in mammalian subjects. In preferred embodiments, the methods are for the detection and/or diagnosis of metastatic melanoma. The mammalian subjects are preferably humans.

The methods comprise a step of detecting the levels of autoantibodies or “autoantibody biomarkers” specifically binding to one or more target antigens in a sample obtained from the mammalian subject. The sample is typically removed from the body such that the analysis of the sample is carried out in vitro. The sample may be any sample known or suspected to contain autoantibodies, as described elsewhere herein.

The autoantibody biomarkers detected in accordance with these further aspects of the invention can be used to detect or diagnose melanoma, particularly metastatic melanoma, on the basis that they are present at higher or lower levels in melanoma patients as compared with healthy controls.

In certain embodiments, the sample is tested for autoantibody biomarkers that bind to one or more of the antigens selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, and NOVA2. Autoantibody biomarkers that bind to one or more of the antigens listed in this group may be considered positive predictive biomarkers on the basis that these autoantibodies have been reported as increased in melanoma patients as compared with healthy controls. For embodiments wherein one or more of the positive predictive biomarkers listed above is analysed, a higher level of autoantibodies in the patient sample as compared with a pre-determined cut-off value is indicative of melanoma.

Alternatively or in addition, the sample is tested for autoantibody biomarkers that bind to one or more of the antigens selected from: SNRPA, NRIP1, UBAP1, TEX264, PLIN2, LAMC1, CENPH, USB1, ABCB8, C15orf48/NMES1, and MAGED1. Autoantibody biomarkers that bind to one or more of the antigens listed in this group may be considered negative predictive biomarkers on the basis that these autoantibodies have been reported as decreased in melanoma patients as compared with healthy controls. For embodiments wherein one or more of the negative predictive biomarkers listed above is analysed, a lower level of autoantibodies in the patient sample as compared with a pre-determined cut-off value is indicative of melanoma.

The sensitivity of the methods may be increased by testing for multiple autoantibodies i.e. autoantibodies that bind to multiple different antigens. In this regard, the sample may be tested for autoantibodies binding to panels of two or more antigens. In certain embodiments, the patient sample is tested for autoantibodies binding to a panel of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more antigens selected from:

RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4 and NOVA2. In certain embodiments, the patient sample is tested for autoantibodies binding to a panel of 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, 35 or 36 antigens selected from RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4 and NOVA2. In certain embodiments, the patient sample is tested for autoantibodies binding to a panel of two or more, three or more, four or more, five or more, six or more, seven or more, eight or more, nine or more, ten or more antigens selected from: SNRPA, NRIP1, UBAP1, TEX264, PLIN2, LAMC1, CENPH, USB1, ABCB8, C15orf48/NMES1 and MAGED1. In certain embodiments, the patient sample is tested for autoantibodies binding to a panel of 2, 3, 4, 5, 6, 7, 8, 9, 10 or 11 antigens selected from SNRPA, NRIP1, UBAP1, TEX264, PLIN2, LAMC1, CENPH, USB1, ABCB8, C15orf48/NMES1 and MAGED1.

The pre-determined cut-off values against which the autoantibody levels are compared, in accordance with these aspects of the invention, will have been calculated or may be calculated based on the analysis of healthy cohorts of mammalian subjects, preferably human subjects.

The pre-determined cut-off value may be different for different autoantibodies. As reported herein, the autoantibody biomarkers used in the methods of these aspects of the invention are either increased or decreased in melanoma patients as compared with healthy controls (see Example 8 and Table 2). As such, these autoantibodies can be analysed in samples obtained from mammalian subjects and the levels compared with pre-determined cut-off values determined for healthy cohorts of subjects so as to detect or diagnose melanoma.

The “healthy cohort” from which the pre-determined cut-off value is calculated for any given autoantibody may be any reasonably-sized cohort of healthy subjects, for example at least 50 subjects, at least 100 subjects, at least 200 subjects, at least 500 subjects. The pre-determined cut-off value against which the autoantibodies of the test sample are compared in accordance with the methods of the invention may be pre-determined based upon a particular healthy cohort matched to the subject under test. For example, the pre-determined cut-off value for autoantibodies binding to any given antigen may be determined on the basis of a healthy cohort matched for any one of the following criteria with the subject under test: age, gender, ethnic origin. The pre-determined cut-off value for any given autoantibody will typically be the average level of autoantibodies calculated for the healthy cohort of mammalian subjects.

Mammalian subjects, particularly humans, tested in accordance with the methods described herein may be any subjects suspected of having melanoma. The subject may be suspected of having melanoma as a result of one or more previous diagnostic tests. The subject may be suspected of having melanoma due to one or more of: family history; carrying alleles or a genotype associated with melanoma; a history of excessive sun exposure; or the existence of moles and/or lesions associated with later development of melanoma. The subject from which the sample is obtained may be a subject who has been diagnosed with melanoma previously and is being monitored for responsiveness to treatment.

The autoantibodies may be detected using any suitable immunoassay technique known to those skilled in the art. A variety of exemplary techniques are described herein and may be employed in accordance with the methods of detection and diagnosis of the invention.

In certain embodiments, the methods comprise the steps of:

(a) contacting the sample obtained from the mammalian subject with the melanoma antigen; and

(b) determining the presence of complexes of the melanoma antigen bound to autoantibodies so as to determine the level of autoantibodies in the sample; and

(c) comparing the level of autoantibodies in the sample with a pre-determined cut-off value.

For panel embodiments i.e. wherein autoantibodies binding to multiple antigens are detected, the methods may involve:

(a) contacting the sample with a panel of two or more antigens;

(b) determining the presence of autoantibody-antigen complexes for each of the antigens so as to determine the level of autoantibodies specifically binding each antigen in the sample; and

(c) comparing the levels of autoantibodies for each antigen with pre-determined cut-off values.

For embodiments wherein the methods involve determining the levels of autoantibodies specifically binding to multiple antigens, melanoma may be detected or diagnosed if the autoantibody levels for at least one of the antigens are higher or lower than the pre-determined cut-off value for autoantibodies specifically binding to that antigen. For embodiments wherein the method involves determining the levels of autoantibodies specifically binding to multiple antigens, as described above, melanoma may be detected or diagnosed if the autoantibody levels for at least two, at least three, at least four, at least five of the antigens are higher or lower than the pre-determined cut-off values for autoantibodies specifically binding to the corresponding antigens. In some embodiments wherein the method involves determining the levels of autoantibodies binding to multiple antigens, melanoma may be detected or diagnosed if the levels of autoantibodies specifically binding to each antigen tested are higher or lower than the pre-determined cut-off values for autoantibodies specifically binding to the corresponding antigens.

The methods of melanoma detection and melanoma diagnosis described herein may comprise an additional step of treating the subject based upon positive detection of disease or a positive diagnosis. The subjects may receive any melanoma treatment known to those skilled in the art including but not limited to surgery, chemotherapy, radiotherapy or other standard of care treatments. In certain embodiments, the subject may be treated with a checkpoint inhibitor including but not limited to ipilimumab, nivolumab, pembrolizumab or a combination thereof.

C. Kits

The present invention further encompasses a kit suitable for performing any one of the methods of the invention, wherein the kit comprises:

(a) one or more melanoma antigens; and

(b) a reagent capable of detecting complexes of the melanoma antigen(s) bound to autoantibodies present in the test sample obtained from the melanoma patient or mammalian subject.

The invention also encompasses a kit for the detection of autoantibodies in a test sample obtained from a mammalian subject, the kit comprising:

(a) one or more melanoma antigens selected from the following:

RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, NOVA2, SNRPA, NRIP1, UBAP1, TEX264, PLIN2, LAMC1, CENPH, USB1, ABCB8, C15orf48/NMES1 and MAGED1; and

(b) a reagent capable of detecting complexes of the melanoma antigen(s) bound to autoantibodies present in the test sample obtained from the mammalian subject.

The invention also encompasses a kit for the detection of autoantibodies in a test sample obtained from a melanoma patient, the kit comprising:

(a) one or more melanoma antigens selected from the following;

ABCB8, ACTB, AKT2, AMPH, AP1S1, AP2B1, AQP4, ARRB1, ATG4D, ATP13A2, BAG6, BCL7B, BICD2, BIRC5, BTBD2, BTRC, C15orf48, C17orf85, CALR, CAP2, CASP10, CASP8, CCDC51, CCNB1, CEACAM5, CENPH, CENPV, CEP131, CFB, CREB3L1, CSNK2A1, CTAG1B, CTSW, CXXC1, DFFA, DHFR, EGFR, EGLN2, EIF4E2, ELMO2, EOMES, ERBB3, FADD, FGA, FGFR1, FLNA, FN1, FOXO1, FRS2, GABARAPL2, GNAI2, GNG12, GPHN, GRAMD4, GRK6, GRP, GSK3A, HDAC1, HES1, HIST2H2AA3, HSPA1B, HSPA2, HSPB1, HSPD1, IGF2BP2, IL3, IL4R, IL17A, IL23A, IL36RN, KDM4A, KLKB1, KRT7, KRT19, L1CAM, LAMB2, LAMC1, LARP1, LEPR, LGALS3BP, MAGEB4, MAGED2, MAPT, MAZ, MIF, MITF, MLLT6, MMP3, MSH2, MUM1, MUC12, NCOA1, NOVA2, NRIP1, OGT, PAPOLG, PCDH1, PDCD6IP, PECAM1, PIAS3, PLIN2, POLR3B, PPL, PPP1R12A, PPP1R2, PRKCI, PTPRR, RALY, RAPGEF3, RELT, RFWD2, RPLP0, RPLP2, RPRM, RPS6KA1, S100A8, S100A14, SDCBP, SHC1, SIGIRR, SIPA1L1, SIVA1, SMAD9, SNRNP70, SNRPA, SNRPD1, SQSTM1, SPA17, SPTB, SPTBN1, SSB, SUFU, SUM02, TEX264, TMEM98, TOLLIP, TONSL, TP53, TPM2, TRAF3IP3, TRIP4, UBAP1, UBE2Z, UBTF, USB1, WHSC1L1, XRCC5 and XRCC6;

and

(b) a reagent capable of detecting complexes of the melanoma antigen(s) bound to autoantibodies present in the test sample obtained from the melanoma patient.

The invention also encompasses a kit for the detection of autoantibodies in a test sample obtained from a melanoma patient, the kit comprising:

(a) one or more melanoma antigens selected from the following;

ABCB8, ACTB, AQP4, ARRB1, ATP13A2, BCL7B, BIRC5, BTRC, C15orf48, C17orf85, CALR, CAP2, CASP10, CCDC51, CCNB1, CEACAM5, CENPH, CENPV, CEP131, CREB3L1, CSNK2A1, CTAG1B, CXXC1, DFFA, DHFR, EGFR, EGLN2, EIF4E2, ELMO2, EOMES, ERBB3, FADD, FLNA, FOXO1, FRS2, GNAI2, GNG12, GRAMD4, GRK6, GSK3A, HDAC1, HES1, HIST2H2AA3, HSPA1B, HSPA2, HSPB1, HSPD1, IGF2BP2, IL17A, IL36RN, KLKB1, LAMB2, LARP1, LGALS3BP, MAPT, MAZ, MLLT6, MMP3, MSH2, MUC12, NOVA2, NRIP1, OGT, PAPOLG, PDCD6IP, PECAM1, PLIN2, POLR3B, PPL, PPP1R12A, PPP1R2, PTPRR, RALY, RAPGEF3, RFWD2, RPRM, RPS6KA1, S100A8, S100A14, SHC1, SIGIRR, SIPA1L1, SIVA1, SMAD9, SNRNP70, SNRPA, SNRPD1, SQSTM1, SPTB, SSB, SUFU, TEX264, TOLLIP, TONSL, TRAF3IP3, TRIP4, UBAP1, USB1, WHSC1L1, XRCC5 and XRCC6;

and

(b) a reagent capable of detecting complexes of the melanoma antigen(s) bound to autoantibodies present in the test sample obtained from the melanoma patient.

In certain embodiments the kit may further comprise:

(c) means for contacting the melanoma antigen with a test sample obtained from the mammalian subject or melanoma patient.

Examples of means for contacting the melanoma antigen with a test sample include the immobilisation of the melanoma antigen on a chip, slide, wells of a microtitre plate, bead, membrane or nanoparticle.

In some embodiments, melanoma antigens within the kit may be present within a panel of two or more melanoma antigens. Within this embodiment the panel may comprise two, three, four, five, six, seven, eight, nine, ten, eleven, twelve, thirteen, fourteen, fifteen, sixteen, seventeen, eighteen, nineteen, twenty, twenty five, thirty, thirty five, forty, forty five or fifty antigens selected from any of the melanoma antigens identified above.

In certain embodiments, the panel of two or more melanoma antigens comprises or consists of SIVA1, IGF2BP2, AQP4, C15orf48, CTAG1B and PAPOLG.

In certain embodiments, the panel of two or more melanoma antigens comprises or consists of HSPA2, SMAD9, HIST2H2AA3 and S100A8.

In certain embodiments, the panel of two or more melanoma antigens comprises or consists of FRS2, BIRC5, EIF3E, CENPH and PAPOLG.

In certain embodiments, the panel of two or more melanoma antigens comprises or consists of POLR3B, ELMO2, RFWD2, SQSTM1, HSPD1 and IL17A.

In certain embodiments, the panel of two or more melanoma antigens comprises or consists of NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2 and IL36RN.

In certain embodiments, the panel of two or more melanoma antigens comprises or consists of CXXC1, LARP1, EGLN2, RPRM, WHSC1L1 and S100A8.

In certain embodiments, the panel of two or more melanoma antigens comprises or consists of SUM02, GRP, SDCBP, AMPH, GPHN, BAG6, BICD2, TMEM98, MUM1, CTSW, NCOA1, MIF, SPA17, FGFR1 and KRT19.

In certain embodiments, the panel of two or more melanoma antigens comprises or consists of NCOA1, MIF, SDCB4, MUM1, FGFR1 and KRT19.

In certain embodiments, the panel of two or more melanoma antigens comprises or consists of MIF, NCOA1, FGFR1 and SDCBP.

In certain embodiments, the panel of two or more melanoma antigens comprises or consists of SUMO2, GRP and MIF.

In certain embodiments, the panel of two or more melanoma antigens comprises or consists of GRK6 and GRAMD4.

In certain embodiments, the panel of two or more melanoma antigens comprises or consists of TEX264, CREB3L1, HSPA1B, SPTB, MUC12, ERBB3, CASP10, FOXO1, FRS2, PPP1R12A and CAP2.

In certain embodiments, the panel of two or more melanoma antigens comprises or consists of GNG12, CCDC51, USB1, GRAMD4, RPS6KA1 and BCL7B.

In certain embodiments, the panel of two or more melanoma antigens comprises or consists of EOMES, CREB3L1, FRS2, PLIN2, SIPA1L1, ABCB8, MAPT, XRCC5, XRCC6, UBAP1, TRIP4 and EIF4E2.

In certain embodiments, the panel of two or more melanoma antigens comprises or consists of S100A14, MMP3, SHC1, CSNK2A1, DFFA, S100A8, HDAC1, MSH2, CEACAM5, DHFR and ARRB1.

In certain embodiments, the panel of two or more melanoma antigens comprises or consists of FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

In certain embodiments, the panel of two or more melanoma antigens comprises or consists of MAGED2, PIAS3, MITF, AP2B1, PRKCI, AKT2, BTBD2, UBE2Z, L1CAM, GABARAPL2, LAMC1, RPLP2, AMPH, AP1S1, LEPR, TP53, IL23A, CFB, FGA, IL3, IL4R, THEM98, KDM4A, UBTF, CASP8, PCDH1, RELT, SPTBN1, RPLP2, KRT7, MUM1, FM1, MAGEB4, CTSW, ATG4D, TPM2 and SPA17.

In certain embodiments, the panel of two or more melanoma antigens comprises or consists of IL4R, L1CAM, MITF, PIAS3, AP1S1, ATG4D and RPLP2.

In certain embodiments, the panel of two or more melanoma antigens comprises or consists of RELT, CASP8, UBE2Z, IL4R, LAMC1, L1CAM and MITF.

In certain embodiments, the panel of two or more melanoma antigens comprises or consists of PIAS3, RPLP2, ATG4D, KRT7, TPM2, GABARAPL2 and MAGEB4.

In certain embodiments, the panel of two or more melanoma antigens comprises or consists of MAGED2, PIAS3, MITF, AP2B1 and PRKC1.

Within the kits of the invention, the patient sample may be selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.

D. Uses

The present invention also encompasses uses of the melanoma antigens described herein in the methods of the invention.

In certain embodiments, encompassed herein is use of one or more melanoma antigens selected from the following: ABCB8, ACTB, AKT2, AMPH, AP1S1, AP2B1, AQP4, ARRB1, ATG4D, ATP13A2, BAG6, BCL7B, BICD2, BIRC5, BTBD2, BTRC, C15orf48, C17orf85, CALR, CAP2, CASP10, CASP8, CCDC51, CCNB1, CEACAM5, CENPH, CENPV, CEP131, CFB, CREB3L1, CSNK2A1, CTAG1B, CTSW, CXXC1, DFFA, DHFR, EGFR, EGLN2, EIF4E2, ELMO2, EOMES, ERBB3, FADD, FGA, FGFR1, FLNA, FN1, FOXO1, FRS2, GABARAPL2, GNAI2, GNG12, GPHN, GRAMD4, GRK6, GRP, GSK3A, HDAC1, HES1, HIST2H2AA3, HSPA1B, HSPA2, HSPB1, HSPD1, IGF2BP2, IL3, IL4R, IL17A, IL23A, IL36RN, KDM4A, KLKB1, KRT7, KRT19, L1CAM, LAMB2, LAMC1, LARP1, LEPR, LGALS3BP, MAGEB4, MAGED2, MAPT, MAZ, MIF, MITF, MLLT6, MMP3, MSH2, MUM1, MUC12, NCOA1, NOVA2, NRIP1, OGT, PAPOLG, PCDH1, PDCD6IP, PECAM1, PIAS3, PLIN2, POLR3B, PPL, PPP1R12A, PPP1R2, PRKCI, PTPRR, RALY, RAPGEF3, RELT, RFWD2, RPLP0, RPLP2, RPRM, RPS6KA1, S100A8, S100A14, SDCBP, SHC1, SIGIRR, SIPA1L1, SIVA1, SMAD9, SNRNP70, SNRPA, SNRPD1, SQSTM1, SPA17, SPTB, SPTBN1, SSB, SUFU, SUM02, TEX264, TMEM98, TOLLIP, TONSL, TP53, TPM2, TRAF3IP3, TRIP4, UBAP1, UBE2Z, UBTF, USB1, WHSC1L1, XRCC5 and XRCC6; in a method for selecting a melanoma patient for treatment with a checkpoint inhibitor wherein the method is performed in accordance with the methods for selecting patients described herein.

In certain embodiments, encompassed herein is use of one or more melanoma antigens selected from the following: ABCB8, ACTB, AQP4, ARRB1, ATP13A2, BCL7B, BIRC5, BTRC, C15orf48, C17orf85, CALR, CAP2, CASP10, CCDC51, CCNB1, CEACAM5, CENPH, CENPV, CEP131, CREB3L1, CSNK2A1, CTAG1B, CXXC1, DFFA, DHFR, EGFR, EGLN2, EIF4E2, ELMO2, EOMES, ERBB3, FADD, FLNA, FOXO1, FRS2, GNAI2, GNG12, GRAMD4, GRK6, GSK3A, HDAC1, HES1, HIST2H2AA3, HSPA1B, HSPA2, HSPB1, HSPD1, IGF2BP2, IL17A, IL36RN, KLKB1, LAMB2, LARP1, LGALS3BP, MAPT, MAZ, MLLT6, MMP3, MSH2, MUC12, NOVA2, NRIP1, OGT, PAPOLG, PDCD6IP, PECAM1, PLIN2, POLR3B, PPL, PPP1R12A, PPP1R2, PTPRR, RALY, RAPGEF3, RFWD2, RPRM, RPS6KA1, S100A8, S100A14, SHC1, SIGIRR, SIPA1L1, SIVA1, SMAD9, SNRNP70, SNRPA, SNRPD1, SQSTM1, SPTB, SSB, SUFU, TEX264, TOLLIP, TONSL, TRAF3IP3, TRIP4, UBAP1, USB1, WHSC1L1, XRCC5 and XRCC6; in a method for selecting a melanoma patient for treatment with a checkpoint inhibitor wherein the method is performed in accordance with the methods for selecting patients described herein.

In certain embodiments, encompassed herein is use of one or more melanoma antigens selected from the following: ACTB, AQP4, ARRB1, BCL7B, BIRC5, C15orf48, C17orf85, CALR, CCDC51, CCNB1, CEACAM5, CENPH, CENPV, CEP131, CSNK2A1, CTAG1B, DFFA, DHFR, EIF3E, EOMES, FGA, FGFR1, FLNA, FRS2, GNAI2, GNG12, GPHN, GRAMD4, GRK6, GSK3A, HDAC1, HES1, IGF2BP2, IL36RN, MAZ, MIF, MLLT6, MMP3, MSH2, NOVA2, NRIP1, PAPOLG, PPP1R2, PTPRR, RALY, RPS6KA1, S100A14, S100A8, SHC1, SIVA1, SNRNP70, SNRPA, SNRPD1, SSB, TEX264, TRAF3IP3, USB1, XRCC5 and XRCC6; in a method for predicting a melanoma patient's responsiveness to treatment with a checkpoint inhibitor wherein the method is performed in accordance with the methods for predicting responsiveness described herein.

In certain embodiments, encompassed herein is use of one or more melanoma antigens selected from the following: ACTB, AQP4, ARRB1, BCL7B, BIRC5, C15orf48, C17orf85, CALR, CCDC51, CCNB1, CEACAM5, CENPH, CENPV, CEP131, CSNK2A1, CTAG1B, DFFA, DHFR, EIF3E, EOMES, FGA, FGFR1, FLNA, FRS2, GNAI2, GNG12, GPHN, GRAMD4, GRK6, GSK3A, HDAC1, HES1, IGF2BP2, IL36RN, MAZ, MIF, MLLT6, MMP3, MSH2, NOVA2, NRIP1, PAPOLG, PPP1R2, PTPRR, RALY, RPS6KA1, S100A14, S100A8, SHC1, SIVA1, SNRNP70, SNRPA, SNRPD1, SSB, TEX264, TRAF3IP3, USB1, XRCC5 and XRCC6; in a method for predicting a melanoma patient's survival responsive to treatment with a checkpoint inhibitor wherein the method is performed in accordance with the methods for predicting survival described herein.

In certain embodiments, encompassed herein is use of one or more melanoma antigens selected from the following: ABCB8, AKT2, AMPH, AP1S1, AP2B1, ARRB1, ATG4D, ATP13A2, BAG6, BICD2, BTBD2, BTRC, CAP2, CASP10, CASP8, CEACAM5, CFB, CREB3L1, CSNK2A1, CTSW, CXXC1, DFFA, DHFR, EGFR, EGLN2, EIF4E2, ELMO2, EOMES, ERBB3, FADD, FGA, FGFR1, FN1, FOXO1, FRS2, GABARAPL2, GPHN, GRP, HDAC1, HIST2H2AA3, HSPA1B, HSPA2, HSPD1, IL17A, IL23A, IL3, IL4R, KDM4A, KLKB1, KRT19, KRT7, L1CAM, LAMB2, LAMC1, LARP1, LEPR, LGALS3BP, MAGEB4, MAGED2, MAPT, MIF, MITF, MMP3, MSH2, MUC12, MUM1, NCOA1, OGT, PCDH1, PDCD6IP, PECAM1, PIAS3, PLIN2, POLR3B, PPL, PPP1R12A, PRKCI, RAPGEF3, RELT, RFWD2, RPLP0, RPLP2, RPRM, S100A14, S100A8, SDCBP, SHC1, SIGIRR, SIPA1L1, SMAD9, SPA17, SPTB, SPTBN1, SQSTM1, SUFU, SUM02, TEX264, TMEM98, TOLLIP, TONSL, TP53, TPM2, TRIP4, UBAP1, UBE2Z, UBTF, WHSC1L1, XRCC5 and XRCC6; in a method for predicting an immune-related adverse event (irAE) in a melanoma patient treated with a checkpoint inhibitor wherein the method is performed in accordance with the methods for predicting irAEs described herein.

In certain embodiments, encompassed herein is use of one or more melanoma antigens selected from the following: ABCB8, ARRB1, ATP13A2, BTRC, CAP2, CASP10, CEACAM5, CREB3L1, CSNK2A1, CXXC1, DFFA, DHFR, EGFR, EGLN2, EIF4E2, ELMO2, EOMES, ERBB3, FADD, FOXO1, FRS2, HDAC1, HIST2H2AA3, HSPA1B, HSPA2, HSPD1, IL17A, KLKB1, LAMB2, LARP1, LGALS3BP, MAPT, MMP3, MSH2, MUC12, OGT, PDCD6IP, PECAM1, PLIN2, POLR3B, PPL, PPP1R12A, RAPGEF3, RFWD2, RPRM, S100A14, S100A8, SHC1, SIGIRR, SIPA1L1, SMAD9, SPTB, SQSTM1, SUFU, TEX264, TOLLIP, TONSL, TRIP4, UBAP1, WHSC1L1, XRCC5 and XRCC6; in a method for predicting an immune-related adverse event (irAE) in a melanoma patient treated with a checkpoint inhibitor wherein the method is performed in accordance with the methods for predicting irAEs described herein.

In certain embodiments, encompassed herein is use of one or more melanoma antigens selected from the following: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, NOVA2, SNRPA, NRIP1, UBAP1, TEX264, PLIN2, LAMC1, CENPH, USB1, ABCB8, C15orf48/NMES1 and MAGED1; in a method for detecting or diagnosing melanoma in a mammalian subject wherein the method is performed in accordance with the methods for detecting and diagnosing melanoma described herein.

### EXAMPLES

The invention will now be further understood with reference to the following non-limiting examples. The use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Production of Recombinant Autoantigens

Recombinant antigens were produced in Escherichia coli. Five cDNA libraries originating from different human tissues (fetal brain, colon, lung, liver, CD4-induced and non-induced T cells) were used for the recombinant production of human antigens. All of these cDNA libraries were oligo(dT)-primed, containing the coding region for an N-terminally located hexa-histidine-tag and were under transcriptional control of the lactose inducible promoter (from E. coli). Sequence integrity of the cDNA libraries was confirmed by 5′ DNA sequencing. Additionally, expression clones representing the full-length sequence derived from the human ORFeome collection were included. Individual antigens were designed in silico, synthesized chemically (Life Technologies, Carlsbad, USA) and cloned into the expression vector pQE30-NST fused to the coding region for the N-terminal-located His6-tag. Recombinant gene expression was performed in E. coli SCS1 cells carrying plasmid pSE111 for improved expression of human genes. Cells were cultivated in 200 ml auto-induction medium (Overnight Express auto-induction medium, Merck, Darmstadt, Germany) overnight and harvested by centrifugation. Bacterial pellets were lysed by resuspension in 15 ml lysis buffer (6 M guanidinium-HCl, 0.1 M NaH₂PO₄, 0.01 M Tris-HCl, pH 8.0).

Soluble proteins were affinity-purified after binding to Protino® Ni-IDA 1000 Funnel Column (Macherey-Nagel, Düren, Germany). Columns were washed with 8 ml washing buffer (8 M urea, 0.1 M NaH₂PO₄, 0.01 M Tris-HCl, pH 6.3). Proteins were eluted in 3 ml elution buffer (6 M urea, 0.1 M NaH₂PO₄, 0.01 M Tris-HCl, 0.5% (w/v) trehalose pH 4.5). Each protein preparation was transferred into 2D-barcoded tubes, lyophilized and stored at −20° C.

### Example 2

### Selection of Antigens and Design of the Cancer Screen

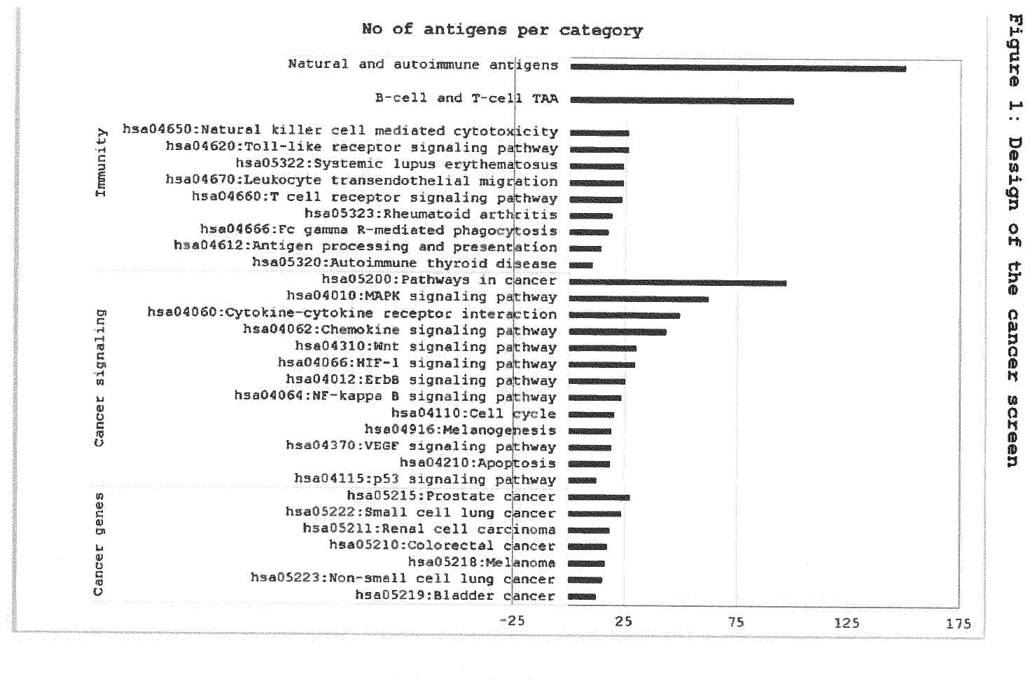

A bead-based array was designed to screen for autoantibodies binding to tumor-associated antigens (TAA), proteins expressed from mutated or overexpressed cancer genes, and proteins playing a role in cancer signaling pathways. Furthermore, self-reactive antigens of normal humans and typical autoimmune antigens were included. In total, 842 potential antigens were selected. FIG. 1 shows the number of screening antigens per category.

### Example 3

### Coupling of Antigens to Beads

For the production of bead-based arrays (BBA), the proteins were coupled to magnetic carboxylated color-coded beads (MagPlex™ microspheres, Luminex Corporation, Austin, Tex., USA). The manufacturer's protocol for coupling proteins to MagPlex™ microspheres was adapted to use liquid handling systems. A semi-automated coupling procedure of one BBA encompassed 384 single, separate coupling reactions, which were carried out in four 96-well plates. For each single coupling reaction, up to 12.5 μg antigen and 8.8×10⁵ MagPlex™ beads of one color region (ID) were used. All liquid handling steps were carried out by either an eight-channel pipetting system (Starlet, Hamilton Robotics, Bonaduz, Switzerland) or a 96-channel pipetting system (Evo Freedom 150, Tecan, Männderdorf, Switzerland). For semi-automated coupling, antigens were dissolved in H₂O, and aliquots of 60 μl were transferred from 2D barcode tubes to 96-well plates. MagPlex™ microspheres were homogeneously resuspended and each bead ID was transferred in one well of a 96-well plate. The 96-well plates containing the microspheres were placed on a magnetic separator (LifeSep™, Dexter Magnetic Technologies Inc., Elk Grove Village, USA) to sediment the beads for washing steps and on a microtiter plate shaker (MTS2/4, IKA) to facilitate permanent mixing for incubation steps.

For coupling, the microspheres were washed three times with activation buffer (100 mM NaH₂PO₄, pH 6.2) and resuspended in 120 μl activation buffer. To obtain reactive sulfo-NHS-ester intermediates, 15 μl 1-ethyl-3-(3-dimethylaminopropyl) carbodiimide (50 mg/ml) and 15 μl N-hydroxy-succinimide (50 mg/ml) were applied to microspheres. After 20 minutes incubation (900 rpm, room temperature (RT)) the microspheres were washed three times with coupling buffer (50 mM MES, pH 5.0) and resuspended in 65 μl coupling buffer. Immediately, 60 μl antigen solution was added to reactive microspheres and coupling took place over 120 minutes under permanent mixing (900 rpm, RT). After three wash cycles using washing buffer (PBS, 0.1% Tween20) coupled beads were resuspended in blocking buffer (PBS, 1% BSA, 0.05% ProClin300), incubated for 20 minutes (900 rpm, RT) and then transferred to be maintained at 4-8° C. for 12-72 h.

Finally, a multiplex BBA was produced by pooling 384 antigen-coupled beads.

### Example 4

### Incubation of Serum Samples with Antigen-Coupled Beads

Serum samples were transferred to 2D barcode tubes and a 1:100 serum dilution was prepared with assay buffer (PBS, 0.5% BSA, 10% E. coli lysate, 50% Low-Cross buffer (Candor Technologies, Nürnberg, Germany)) in 96-well plates. The serum dilutions were first incubated for 20 minutes to neutralize any human IgG eventually directed against E. coli proteins. The BBA was sonicated for 5 minutes and the bead mix was distributed in 96-well plates. After three wash cycles with washing buffer (PBS, 0.05% Tween20) serum dilutions (50 μl) were added to the bead mix and incubated for 20 h (900 rpm, 4-8° C.). Supernatants were removed from the beads by three wash cycles, and secondary R-phycoerythrin-labeled antibody (5 μg/ml, goat anti-human, Dianova, Hamburg, Germany) was added for a final incubation of 45 minutes (900 rpm, RT). The beads were washed three times with washing buffer (PBS, 0.1% Tween20) and resuspended in 100 μl sheath fluid (Luminex Corporation). Subsequently, beads were analyzed in a FlexMap3D device for fluorescent signal readout (DD gate 7.500-15.000; sample size: 80 μl; 1000 events per bead ID; timeout 60 sec). The binding events were displayed as median fluorescence intensity (MFI). Measurements were disregarded when low numbers of bead events (<30 beads) were counted per bead ID.

### Example 5

### Statistical Analysis

Statistical analysis was performed to identify biomarkers associated with the effectiveness and side effects of cancer immunotherapy. Autoantibody levels were correlated with overall survival (OS), progression-free survival (PFS), and immune-related adverse events (irAEs) using Spearman's rank correlation test. In the case, when two groups were compared the permutation based statistical technique Significance of microarrays in the R-programming language (SAMR) was used (Tusher et al., 2001). The strength of differences between the two test groups is computed as SAMR score_d. Furthermore, receiver-operating characteristics were calculated to provide area under the curve (AUC) values for each antigen. The ROC curves were generated using the package pROC (Robin et al., 2011).

To evaluate the tumor response to treatment, the best overall response (BOR) was determined by RECIST v1.1 criteria and the disease control rate (DCR) was calculated. The DCR is the percentage of patients achieving complete response (CR), or partial response (PR) or stable disease (SD). To identify biomarkers that predict clinical response in pre-treatment samples (T0), a responder was defined as with CR, PR, or SD and autoantibody profiles of patients with DCR compared to patients with progressive disease (PD).

### Example 6

### Collection of Serum Samples from Patients with Metastatic Melanoma Treated with Different Immune Checkpoint Inhibitors

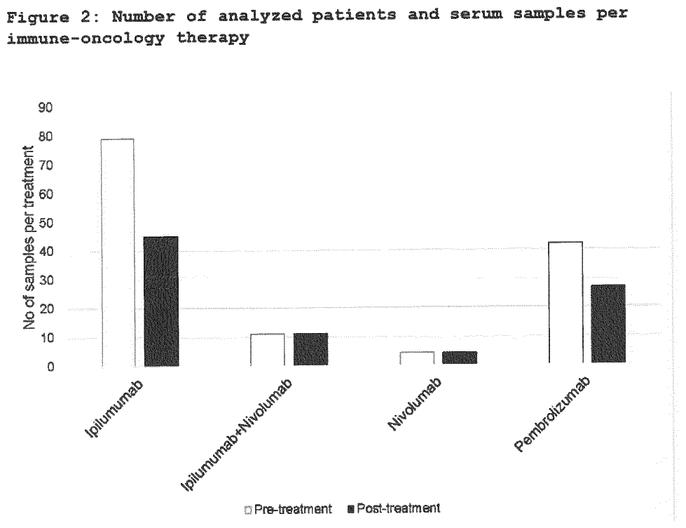

Serum samples of metastatic melanoma patients treated with immune checkpoint inhibitors were collected at the National Center for Tumor Diseases (NCT, Heidelberg, Germany). Serum samples were collected prior to immune checkpoint inhibitor treatment (T0, baseline or pre-treatment sample) and at two time points during treatment (post-treatment samples). The T1 samples correspond to 90 days (3 month) and the T2 samples correspond to 180 days (6 month). FIG. 2 shows the number of patients and samples per treatment group.

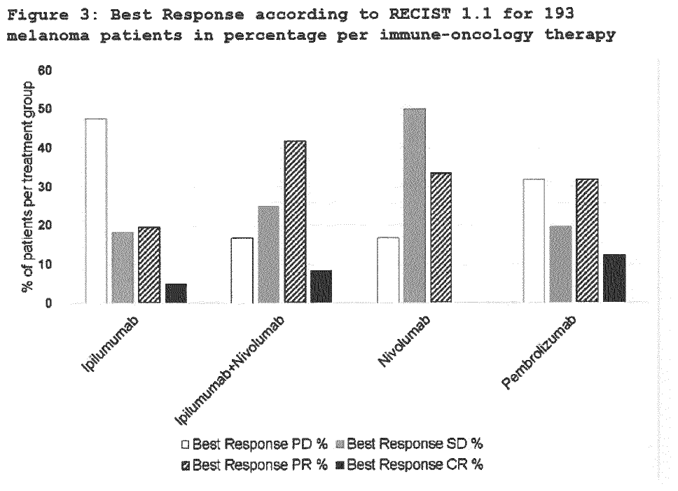

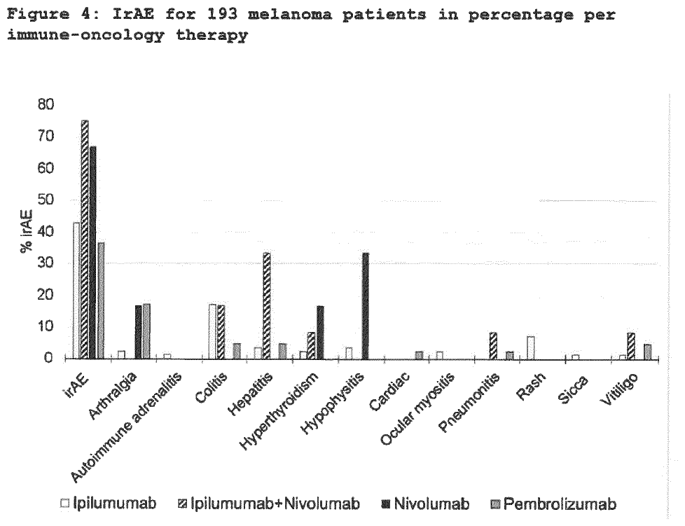

Patient data were provided on a standardized form including demographics (age, gender), the type of checkpoint inhibitor treatment, the date of therapy start, and best response according to “Response Evaluation Criteria in Solid tumors” (RECIST 1.1. criteria), graded into complete response (CR), partial response (PR), stable disease (SD), and progressive disease (PD) (Eisenhauer et al., 2009). FIG. 3 shows the response categories (CR, PR, SD, and PD) achieved by patients treated with different checkpoint inhibitors.

Furthermore, details on immune-related adverse events (irAE) were recorded. FIG. 4 shows the different irAEs, which occurred following treatment with different checkpoint inhibitors. The highest percentage (75%) of irAEs occurred during ipilimumab/nivolumab combination therapy.

Colitis most frequently occurred during ipilimumab and ipilimumab/nivolumab combination therapy.

The survival time (overall survival, OS) was calculated as the time from start of treatment to death or the last contact date. Progression-free survival (PFS) was calculated as the time from start of treatment to progression. When progression was not observed, the time from start to death or last visit was calculated.

### Example 7

### Characterization of the Autoantibody Response in Melanoma Patients

The presence of a tumor can induce a humoral immune response to tumor-associated antigens (TAA) and self-antigens. This autoantibody response may be utilized to characterize the immune-status of a cancer patient receiving immune-oncology therapy. Pre- and post-treatment serum samples from 193 melanoma patients treated with anti-CTLA-4 (ipilimumab), anti-PD-1 (nivolumab or pembrolizumab) or anti-CTLA-4/anti-PD-1 combination therapy were analyzed for the presence of autoantibodies directed towards 842 preselected tumor-associated antigens (TAA) and self-antigens.

Table 1 shows the autoantibody response of melanoma patients against 135 antigens. Markers correlating with different clinical endpoints are extracted and shown in separate tables (T). Table 1 includes the following antigens:

GRAMD4, TEX264, CREB3L1, NCBP3/C17orf85, FRS2, S100A8, TRAF3IP3, NOVA2, C15orf48; NMES1, MIF, CTAG1B, CAP2, CSNK2A1, IGF2BP2, GPHN, SDCBP, HSPA1B, SPTB, HES1, MMP3, PAPOLG, SNRPD1, SSB, XRCC5, XRCC6, EOMES, ERBB3, ATG4D, ELMO2, AKAP13, HSPA2, SMAD9, BIRC5, FGA, PDCD6IP, RPS6KA1, USB1, BCL7B, EIF3E, CENPH, GNG12, CCDC51, HUS1, HSPB1, KLKB1, LARP1, LGALS3BP, OGT, PECAM1, NRIP1, PPP1R2, IL36RN, RALY, S100A14, SNRNP70, SNRPA, MUC12, HIST2H2AA3, SIVA1, AQP4, RPLP2, SDC1, TRA2B, EGLN2, RAPGEF3, RPRM, NSD3/WHSC1L1, ATP13A2, CTSW, CXXC1, FADD, ACTB, MLLT6, ARRB1, CEACAM5, GSK3A, HDAC1, LAMC1, MSH2, MAZ, PTPRR, DFFA, DHFR, FLNA, CCNB1, SHC1, CALR, GRK6, GNAI2, FGFR1, CENPV, CEP131, PPP1R12A, CASP10, FOXO1, CPSF1, GRK2, AKT3, ANXA4, ATP1B3, BCR, CDR2L, NME1, CXCL13, CXCL5, DNAJC8, DUSP3, EEF2, MAGED1, EIF4E2, HSPD1, IL17A, MAPT, POLR3B, SIPA1L1, SUMO2, TRIP4, UBAP1, BTRC, EGFR, FN1, KRT7, LAMB2, MITF, PPL, SIGIRR, SPA17, SUFU, TOLLIP, TONSL, PLIN2, RFWD2, ABCB8, SQSTM1, and CTAG2.

TRA2B was tested as a post-translationally modified protein, in which the amino acid arginine was modified by citrullination or deamination into the amino acid citrulline. The modified protein is referred to as “TRA2B_cit”. Autoantibodies binding to citrullinated antigens or peptides (ACPA) are found in rheumatoid arthritis (RA).

TABLE 1
List of all identified antigens

| | | Gene Symbol | | | | | | | | |
|---|---|---|---|---|---|---|---|---|---|---|
| | | and | | | | | | | | |
| | | Exemplary | | | | | | | | |
| | Gene | Antigen | | T | T | T | T | T | T | T |
| ID | ID | Sequence | Gene Name | 2 | 3 | 4 | 5 | 6 | 7 | 8 |
| 1 | 23151 | GRAMD4 (SEQ ID | GRAM domain | | x | | x | | | |
| | | NO: 1) | containing 4 | | | | | | | |
| 2 | 51368 | TEX264 (SEQ ID | testis expressed 264 | x | | x | | | x | x |
| | | NO: 2) | | | | | | | | |
| 3 | 90993 | CREB3L1 (SEQ | cAMP responsive | x | | x | | x | | |
| | | ID NO: 3) | element binding protein | | | | | | | |
| | | | 3-like 1 | | | | | | | |
| 4 | 55421 | NCBP3; C17orf85 | nuclear cap binding | | | | | | x | |
| | | (SEQ ID NO: 4) | subunit 3 | | | | | | | |
| 5 | 10818 | FRS2(SEQ ID NO: | fibroblast growth factor | | | x | x | x | | |
| | | 5) | receptor substrate 2 | | | | | | | |
| 6 | 6279 | S100A8(SEQ ID | S100 calcium binding | | | x | | | x | x |
| | | NO: 6) | protein A8 | | | | | | | |
| 7 | 80342 | TRAF3IP3(SEQ ID | TRAF3 interacting | | | | | | x | |
| | | NO: 7) | protein 3, - | | | | | | | |
| 8 | 4858 | NOVA2(SEQ ID | neuro-oncological | x | | | | | x | |
| | | NO: 8) | ventral antigen 2 | | | | | | | |
| 9 | 84419 | C15orf48; | chromosome 15 open | x | x | | | | x | |
| | | NMES1(SEQ ID | reading frame 48 | | | | | | | |
| | | NO: 9) | | | | | | | | |
| 10 | 4282 | MIF(SEQ ID NO: | macrophage migration | x | x | | | | | x |
| | | 10) | inhibitory factor | | | | | | | |
| | | | (glycosylation-inhibiting | | | | | | | |
| | | | factor) | | | | | | | |
| 11 | 1485 | CTAG1B(SEQ ID | cancer/testis antigen 1B | x | x | | | | | |
| | | NO: 11) | | | | | | | | |
| 12 | 10486 | CAP2(SEQ ID NO: | CAP, adenylate cyclase- | x | | x | | | | x |
| | | 12) | associated protein, 2 | | | | | | | |
| | | | (yeast) | | | | | | | |
| 13 | 1457 | CSNK2A1(SEQ ID | casein kinase 2, alpha 1 | x | | | | | x | |
| | | NO: 13) | polypeptide | | | | | | | |
| 14 | 10644 | IGF2BP2(SEQ ID | insulin-like growth factor | | x | | | | x | |
| | | NO: 14) | 2 mRNA binding protein | | | | | | | |
| | | | 2 | | | | | | | |
| 15 | 10243 | GPHN | gephyrin | x | x | | x | | | |
| | | (SEQ ID | | | | | | | | |
| | | NO: 15) | | | | | | | | |
| 16 | 6386 | SDCBP | syndecan binding protein | | | x | | x | | |
| | | (SEQ ID | (syntenin) | | | | | | | |
| | | NO: 16) | | | | | | | | |
| 17 | 3304 | HSPA1B | heat shock 70 kDa | | | x | | | | x |
| | | (SEQ ID | protein 1B | | | | | | | |
| | | NO: 17) | | | | | | | | |
| 18 | 6710 | SPTB | spectrin, beta, | | | x | | | | x |
| | | (SEQ ID | erythrocytic | | | | | | | |
| | | NO: | | | | | | | | |
| | | 18) | | | | | | | | |
| 19 | 3280 | HES1 | hes family bHLH | | | | | | x | |
| | | (SEQ ID | transcription factor 1 | | | | | | | |
| | | NO: | | | | | | | | |
| | | 19) | | | | | | | | |
| 20 | 4314 | MMP3 | stromelysin 1 | | | | | | x | |
| | | (SEQ ID | | | | | | | | |
| | | NO: 20) | | | | | | | | |
| 21 | 64895 | PAPOLG | poly(A) polymerase | | x | | x | | | |
| | | (SEQ ID | gamma | | | | | | | |
| | | NO: 21) | | | | | | | | |
| 22 | 6632 | SNRPD1 | small nuclear | | | | | | x | |
| | | (SEQ ID | ribonucleoprotein D1 | | | | | | | |
| | | NO: 22) | polypeptide | | | | | | | |
| 23 | 6741 | SSB | Sjogren syndrome | | | | | | x | |
| | | (SEQ ID | antigen B | | | | | | | |
| | | NO: | | | | | | | | |
| | | 23) | | | | | | | | |
| 24 | 7520 | XRCC5 | X-ray repair cross | | | | | x | x | |
| | | (SEQ ID | complementing 5 | | | | | | | |
| | | NO: 24) | | | | | | | | |
| 25 | 2547 | XRCC6 | X-ray repair cross | | | | | x | x | |
| | | (SEQ ID | complementing 6 | | | | | | | |
| | | NO: 25) | | | | | | | | |
| 26 | 8320 | EOMES | eomesodermin | | | | | x | x | |
| | | (SEQ ID | | | | | | | | |
| | | NO: 26) | | | | | | | | |
| 27 | 2065 | ERBB3 | erb-b2 receptor tyrosine | x | | x | | | | x |
| | | (SEQ ID | kinase 3 | | | | | | | |
| | | NO: 27) | | | | | | | | |
| 28 | 84971 | ATG4D | autophagy related 4D, | | | x | | x | | |
| | | (SEQ ID | cysteine peptidase | | | | | | | |
| | | NO: 28) | | | | | | | | |
| 29 | 63916 | ELM02 | engulfment and cell | | | | | x | | x |
| | | (SEQ ID | motility 2 | | | | | | | |
| | | NO: 29) | | | | | | | | |
| 30 | 11214 | AKAP13 | A kinase (PRKA) anchor | x | | | | | | |
| | | (SEQ ID | protein 13 | | | | | | | |
| | | NO: 30) | | | | | | | | |
| 31 | 3306 | HSPA2 | heat shock 70 kDa | | | x | | | | |
| | | (SEQ ID | protein 2 | | | | | | | |
| | | NO: 31) | | | | | | | | |
| 32 | 4093 | SMAD9 | SMAD family member 9 | | | x | | | | |
| | | (SEQ ID | | | | | | | | |
| | | NO: 32) | | | | | | | | |
| 33 | 332 | BIRC5 | baculoviral IAP repeat | | | | x | | | |
| | | (SEQ ID | containing 5 | | | | | | | |
| | | NO: 33) | | | | | | | | |
| 34 | 2243 | FGA | fibrinogen alpha chain | | | | | | x | |
| | | (SEQ ID | | | | | | | | |
| | | NO: | | | | | | | | |
| | | 34) | | | | | | | | |
| 35 | 10015 | PDCD6IP | programmed cell death 6 | | | | | | | x |
| | | (SEQ ID | interacting protein | | | | | | | |
| | | NO: 35) | | | | | | | | |
| 36 | 6195 | RPS6KA1 | ribosomal protein S6 | | | | x | | | |
| | | (SEQ ID | kinase, 90 kDa, | | | | | | | |
| | | NO: 36) | polypeptide 1 | | | | | | | |
| 37 | 79650 | USB1 | U6 snRNA biogenesis 1 | x | | | x | | | |
| | | (SEQ ID | | | | | | | | |
| | | NO: | | | | | | | | |
| | | 37) | | | | | | | | |
| 38 | 9275 | BCL7B | B-cell CLL/lymphoma 7B | | | | x | | | |
| | | (SEQ ID | | | | | | | | |
| | | NO: 38) | | | | | | | | |
| 39 | 3646 | EIF3E | eukaryotic translation | | | | x | | | |
| | | (SEQ ID | initiation factor 3, subunit | | | | | | | |
| | | NO: 39) | E | | | | | | | |
| 40 | 64946 | CENPH | centromere protein H | x | | | x | | | |
| | | (SEQ ID | | | | | | | | |
| | | NO: 40) | | | | | | | | |
| 41 | 55970 | GNG12 | guanine nucleotide | | | | x | | | |
| | | (SEQ ID | binding protein (G | | | | | | | |
| | | NO: 41) | protein), gamma 12 | | | | | | | |
| 42 | 79714 | CCDC51 | coiled-coil domain | | | | x | | | |
| | | (SEQ ID | containing 51 | | | | | | | |
| | | NO: 42) | | | | | | | | |
| 43 | 3364 | HUS1 | HUS1 checkpoint | | | | x | | | |
| | | (SEQ ID | homolog (S. pombe) | | | | | | | |
| | | NO: 43) | | | | | | | | |
| 44 | 3315 | HSPB1 | heat shock 27 kDa | | | | | | | x |
| | | (SEQ ID | protein 1 | | | | | | | |
| | | NO: 44) | | | | | | | | |
| 45 | 3818 | KLKB1 | kallikrein B, plasma | | | | | | | x |
| | | (SEQ ID | (Fletcher factor) 1 | | | | | | | |
| | | NO: 45) | | | | | | | | |
| 46 | 23367 | LARP1 | La ribonucleoprotein | | | | | | | x |
| | | (SEQ ID | domain family, member | | | | | | | |
| | | NO: 46) | 1 | | | | | | | |
| 47 | 3959 | LGALS3BP | lectin, galactoside- | | | | | | | x |
| | | (SEQ | binding, soluble, 3 | | | | | | | |
| | | ID | binding protein | | | | | | | |
| | | NO: 47) | | | | | | | | |
| 48 | 8473 | OGT | O-linked N- | | | | | | | x |
| | | (SEQ ID | acetylglucosamine | | | | | | | |
| | | NO: | (GlcNAc) transferase | | | | | | | |
| | | 48) | | | | | | | | |
| 49 | 5175 | PECAM1 | platelet/endothelial cell | | | | | | | x |
| | | (SEQ ID | adhesion molecule 1 | | | | | | | |
| | | NO: 49) | | | | | | | | |
| 50 | 8204 | NRIP1 | nuclear receptor | x | | | | | x | |
| | | (SEQ ID | interacting protein 1 | | | | | | | |
| | | NO: 50) | | | | | | | | |
| 51 | 5504 | PPP1 R2 | protein phosphatase 1, | | | | | | x | |
| | | (SEQ ID | regulatory (inhibitor) | | | | | | | |
| | | NO: 51) | subunit 2 | | | | | | | |
| 52 | 26525 | IL36RN | interleukin 36 receptor | | | | | | x | |
| | | (SEQ ID | antagonist | | | | | | | |
| | | NO: 52) | | | | | | | | |
| 53 | 22913 | RALY | RALY heterogeneous | | | | | | x | |
| | | (SEQ ID | nuclear | | | | | | | |
| | | NO: | ribonucleoprotein | | | | | | | |
| | | 53) | | | | | | | | |
| 54 | 57402 | S100A14 | S100 calcium binding | | | | | | x | |
| | | (SEQ ID | protein A14 | | | | | | | |
| | | NO: 54) | | | | | | | | |
| 55 | 6625 | SNRNP70 | small nuclear | | | | | | x | |
| | | (SEQ ID | ribonucleoprotein U1 | | | | | | | |
| | | NO: 55) | subunit 70 | | | | | | | |
| 56 | 6626 | SNRPA | small nuclear | x | | | | | x | |
| | | (SEQ ID | ribonucleoprotein | | | | | | | |
| | | NO: 56) | polypeptide A | | | | | | | |
| 57 | 10071 | MUC12 | mucin 12, cell surface | | | x | | | | |
| | | (SEQ ID | associated | | | | | | | |
| | | NO: 57) | | | | | | | | |
| 58 | 8337 | HIST2H2AA3 | histone cluster 2, H2aa3 | | | x | | | | |
| | | (SEQ | | | | | | | | |
| | | ID | | | | | | | | |
| | | NO: 58) | | | | | | | | |
| 59 | 10572 | SIVA1 | SIVA1, apoptosis- | | x | | | | | |
| | | (SEQ ID | inducing factor | | | | | | | |
| | | NO: 59) | | | | | | | | |
| 60 | 361 | AQP4 | aquaporin 4 | x | x | | | | | |
| | | (SEQ ID | | | | | | | | |
| | | NO: 60) | | | | | | | | |
| 61 | 6181 | RPLP2 | ribosomal protein, large, | x | | | | | | |
| | | (SEQ ID | P2 | | | | | | | |
| | | NO: 61) | | | | | | | | |
| 62 | 6382 | SDC1 | syndecan 1 | x | | | | | | |
| | | (SEQ ID | | | | | | | | |
| | | NO: 62) | | | | | | | | |
| 63 | 6434 | TRA2B_cit | transformer 2 beta | x | | | | | | |
| | | (SEQ | homolog | | | | | | | |
| | | ID | (Drosophila) | | | | | | | |
| | | NO: 63) | | | | | | | | |
| 64 | 112398 | EGLN2 | egl-9 family hypoxia- | | | | | | | x |
| | | (SEQ ID | inducible factor 2 | | | | | | | |
| | | NO: 64) | | | | | | | | |
| 65 | 10411 | RAPGEF3 | Rap guanine nucleotide | | | | | | | x |
| | | (SEQ ID | exchange factor | | | | | | | |
| | | NO: 65) | (GEF) 3 | | | | | | | |
| 66 | 56475 | RPRM | reprimo, TP53 | | | | | | | x |
| | | (SEQ ID | dependent G2 arrest | | | | | | | |
| | | NO: 66) | mediator candidate | | | | | | | |
| 67 | 54904 | NSD3; | nuclear receptor binding | | | | | | | x |
| | | WHSC1 L1 | SET domain protein 3 | | | | | | | |
| | | (SEQ | | | | | | | | |
| | | ID | | | | | | | | |
| | | NO: 67) | | | | | | | | |
| 68 | 23400 | ATP13A2 | ATPase type 13A2 | | | | | | | x |
| | | (SEQ ID | | | | | | | | |
| | | NO: 68) | | | | | | | | |
| 69 | 1521 | CTSW | cathepsin W | | | | | | | x |
| | | (SEQ ID | | | | | | | | |
| | | NO: 69) | | | | | | | | |
| 70 | 30827 | CXXC1 | CXXC finger protein 1 | | | | | | | x |
| | | (SEQ ID | | | | | | | | |
| | | NO: 70) | | | | | | | | |
| 71 | 8772 | FADD | Fas | | | | | | | x |
| | | (SEQ ID | (TNFRSF6)- | | | | | | | |
| | | NO: 71) | associated via death | | | | | | | |
| | | | domain | | | | | | | |
| 72 | 60 | ACTB | actin, beta | x | | | | | x | |
| | | (SEQ ID | | | | | | | | |
| | | NO: 72) | | | | | | | | |
| 73 | 4302 | MLLT6 | myeloid/lymphoid or | x | | | | | x | |
| | | (SEQ ID | mixed-lineage leukemia | | | | | | | |
| | | NO: 73) | | | | | | | | |
| 74 | 408 | ARRB1 | arrestin, beta 1 | x | | | | | x | |
| | | (SEQ ID | | | | | | | | |
| | | NO: 74) | | | | | | | | |
| 75 | 1048 | CEACAM5 | carcinoembryonic | | | | | | x | |
| | | (SEQ | antigen-related cell | | | | | | | |
| | | ID | adhesion molecule 5 | | | | | | | |
| | | NO: 75) | | | | | | | | |
| 76 | 2931 | GSK3A | glycogen synthase | | | | | | x | |
| | | (SEQ ID | kinase 3 alpha | | | | | | | |
| | | NO: 76) | | | | | | | | |
| 77 | 3065 | HDAC1 | histone deacetylase 1 | | | | | | x | |
| | | (SEQ ID | | | | | | | | |
| | | NO: 77) | | | | | | | | |
| 78 | 3915 | LAMC1 | laminin, gamma 1 | x | | | | | x | |
| | | (SEQ ID | | | | | | | | |
| | | NO: 78) | | | | | | | | |
| 79 | 4436 | MSH2 | mutS homolog 2 | | | | | | x | |
| | | (SEQ ID | | | | | | | | |
| | | NO: 79) | | | | | | | | |
| 80 | 4150 | MAZ | MYC-associated zinc | | | | | | x | |
| | | (SEQ ID | finger protein | | | | | | | |
| | | NO: | (purine- | | | | | | | |
| | | 80) | binding transcription | | | | | | | |
| | | | factor) | | | | | | | |
| 81 | 5801 | PTPRR | protein tyrosine | | | | | | x | |
| | | (SEQ ID | phosphatase, receptor | | | | | | | |
| | | NO: 81) | type, R | | | | | | | |
| 82 | 1676 | DFFA | DNA fragmentation | | | | | | x | |
| | | (SEQ ID | factor, 45 kDa, alpha | | | | | | | |
| | | NO: | polypeptide | | | | | | | |
| | | 82) | | | | | | | | |
| 83 | 1719 | DHFR | dihydrofolate reductase | | | | | | x | |
| | | (SEQ ID | | | | | | | | |
| | | NO: 83) | | | | | | | | |
| 84 | 2316 | FLNA | filamin A, alpha | | | | | | x | |
| | | (SEQ ID | | | | | | | | |
| | | NO: | | | | | | | | |
| | | 84) | | | | | | | | |
| 85 | 891 | CCNB1 | cyclin B1 | | | | | | x | |
| | | (SEQ ID | | | | | | | | |
| | | NO: 85) | | | | | | | | |
| 86 | 6464 | SHC1 | SHC | x | | | | | x | |
| | | (SEQ ID | (Src homology 2 | | | | | | | |
| | | NO: 86) | domain containing) | | | | | | | |
| | | | transforming protein 1 | | | | | | | |
| 87 | 811 | CALR | calreticulin | | | | | | x | |
| | | (SEQ ID | | | | | | | | |
| | | NO: 87) | | | | | | | | |
| 88 | 2870 | GRK6(SEQ ID | G protein-coupled | x | x | | | | | |
| | | NO: 88) | receptor kinase 6 | | | | | | | |
| 89 | 2771 | GNAI2(SEQ ID | G protein subunit alpha | | | | | | x | |
| | | NO: 89) | i2 | | | | | | | |
| 90 | 2260 | FGFR1(SEQ ID | fibroblast growth factor | | x | | | | | |
| | | NO: 90) | receptor 1 | | | | | | | |
| 91 | 201161 | CENPV(SEQ ID | centromere protein V | | | | | | x | |
| | | NO: 91) | | | | | | | | |
| 92 | 22994 | CEP131(SEQ ID | centrosomal protein | | | | | | x | |
| | | NO: 92) | 131 kDa | | | | | | | |
| 93 | 4659 | PPP1R12A(SEQ | protein phosphatase 1, | | | x | | | | |
| | | ID NO: 93) | regulatory subunit 12A | | | | | | | |
| 94 | 843 | CASP10(SEQ ID | caspase 10 | | | x | | | | |
| | | NO: 94) | | | | | | | | |
| 95 | 2308 | FOXO1(SEQ ID | forkhead box O1 | | | x | | | | |
| | | NO: 95) | | | | | | | | |
| 96 | 29894 | CPSF1 (SEQ ID | cleavage and | x | | | | | | |
| | | NO: 96) | polyadenylation specific | | | | | | | |
| | | | factor 1,160 kDa | | | | | | | |
| 97 | 156 | GRK2(SEQ ID | G protein-coupled | x | | | | | | |
| | | NO: 97) | receptor kinase 2 | | | | | | | |
| 98 | 10000 | AKT3(SEQ ID NO: | v-akt murine thymoma | x | | | | | | |
| | | 98) | viral oncogene homolog | | | | | | | |
| | | | 3 | | | | | | | |
| 99 | 307 | ANXA4(SEQ ID | annexin A4 | x | | | | | | |
| | | NO: 99) | | | | | | | | |
| 100 | 483 | ATP1B3(SEQ ID | ATPase, Na+/K+ | x | | | | | | |
| | | NO: 100) | transporting, beta 3 | | | | | | | |
| | | | polypeptide | | | | | | | |
| 101 | 613 | BCR(SEQ ID NO: | breakpoint cluster region | x | | | | | | |
| | | 101) | | | | | | | | |
| 102 | 30850 | CDR2L(SEQ ID | cerebellar degeneration- | x | | | | | | |
| | | NO: 102) | related protein 2-like | | | | | | | |
| 103 | 4830 | NME1(SEQ ID | NME/NM23 nucleoside | x | | | | | | |
| | | NO: 103) | diphosphate kinase 1 | | | | | | | |
| 104 | 10563 | CXCL13(SEQ ID | chemokine (C-X-C motif) | x | | | | | | |
| | | NO: 104) | ligand 13 | | | | | | | |
| 105 | 6374 | CXCL5(SEQ ID | chemokine (C-X-C motif) | x | | | | | | |
| | | NO: 105) | ligand 5 | | | | | | | |
| 106 | 22826 | DNAJC8(SEQ ID | DnaJ (Hsp40) homolog, | x | | | | | | |
| | | NO: 106) | subfamily C, member 8 | | | | | | | |
| 107 | 1845 | DUSP3(SEQ ID | dual specificity | x | | | | | | |
| | | NO: 107) | phosphatase 3 | | | | | | | |
| 108 | 1938 | EEF2(SEQ ID NO: | eukaryotic translation | x | | | | | | |
| | | 108) | elongation factor 2 | | | | | | | |
| 109 | 9500 | MAGED1(SEQ ID | melanoma antigen family | x | | | | | | |
| | | NO: 109) | D, 1 | | | | | | | |
| 110 | 9470 | EIF4E2(SEQ ID | eukaryotic translation | | | | | x | | |
| | | NO: 110) | initiation factor 4E family | | | | | | | |
| | | | member 2 | | | | | | | |
| 111 | 3329 | HSPD1 (SEQ ID | heat shock protein family | | | | | x | | |
| | | NO: 111) | D (Hsp60) member 1 | | | | | | | |
| 112 | 3605 | IL17A(SEQ ID NO: | interleukin 17A | | | | | x | | |
| | | 112) | | | | | | | | |
| 113 | 4137 | MAPT(SEQ ID | microtubule-associated | | | | | x | | |
| | | NO: 113) | protein tau | | | | | | | |
| 114 | 55703 | POLR3B(SEQ ID | polymerase (RNA) III | | | | | x | | |
| | | NO: 114) | (DNA directed) | | | | | | | |
| | | | polypeptide B | | | | | | | |
| 115 | 26037 | SIPA1L1(SEQ ID | signal-induced | x | | | | x | | |
| | | NO: 115) | proliferation-associated | | | | | | | |
| | | | 1 like 1 | | | | | | | |
| 116 | 6613 | SUMO2(SEQ ID | small ubiquitin-like | | | | | x | | |
| | | NO: 116) | modifier 2 | | | | | | | |
| 117 | 9325 | TRIP4(SEQ ID | thyroid hormone | | | | | x | | |
| | | NO: 117) | receptor interactor 4 | | | | | | | |
| 118 | 51271 | UBAP1 | ubiquitin associated | x | | | | x | | |
| | | (SEQ ID | protein 1 | | | | | | | |
| | | NO: 118) | | | | | | | | |
| 119 | 8945 | BTRC | beta-transducin repeat | x | | | | | | x |
| | | (SEQ ID | containing E3 ubiquitin | | | | | | | |
| | | NO: 119) | protein ligase | | | | | | | |
| 120 | 1956 | EGFR | epidermal growth factor | | | | | | | x |
| | | (SEQ ID | receptor | | | | | | | |
| | | NO: 120) | | | | | | | | |
| 121 | 2335 | FN1 | fibronectin 1 | | | | | | | x |
| | | (SEQ ID | | | | | | | | |
| | | NO: | | | | | | | | |
| | | 121) | | | | | | | | |
| 122 | 3855 | KRT7 | keratin 7, type II | | | | | | | x |
| | | (SEQ ID | | | | | | | | |
| | | NO: | | | | | | | | |
| | | 122) | | | | | | | | |
| 123 | 3913 | LAMB2 | laminin, beta 2 (laminin | | | | | | | x |
| | | (SEQ ID | S) | | | | | | | |
| | | NO: 123) | | | | | | | | |
| 124 | 4286 | MITF | microphthalmia- | | | | | | | x |
| | | (SEQ ID | associated transcription | | | | | | | |
| | | NO: | factor | | | | | | | |
| | | 124) | | | | | | | | |
| 125 | 5493 | PPL | periplakin | | | | | | | x |
| | | (SEQ ID | | | | | | | | |
| | | NO: | | | | | | | | |
| | | 125) | | | | | | | | |
| 126 | 59307 | SIGIRR | single immunoglobulin | x | | | | | | x |
| | | (SEQ ID | and toll-interleukin 1 | | | | | | | |
| | | NO: 126) | receptor (TIR) domain | | | | | | | |
| 127 | 53340 | SPA17 | sperm autoantigenic | | | | | | | x |
| | | (SEQ ID | protein 17 | | | | | | | |
| | | NO: 127) | | | | | | | | |
| 128 | 51684 | SUFU | suppressor of fused | x | | | | | | x |
| | | (SEQ ID | homolog (Drosophila) | | | | | | | |
| | | NO: 128) | | | | | | | | |
| 129 | 54472 | TOLLIP | toll interacting protein | | | | | | | x |
| | | (SEQ ID | | | | | | | | |
| | | NO: 129) | | | | | | | | |
| 130 | 4796 | TONSL | tonsoku-like, DNA repair | | | | | | | x |
| | | (SEQ ID | protein | | | | | | | |
| | | NO: 130) | | | | | | | | |
| 131 | 123 | PLIN2 | perilipin 2 | x | | | | x | | |
| | | (SEQ ID | | | | | | | | |
| | | NO: 131) | | | | | | | | |
| 132 | 64326 | RFWD2 | ring finger and WD | | | | | x | | |
| | | (SEQ ID | | | | | | | | |
| | | NO: 132) | repeat domain 2, E3 | | | | | | | |
| | | | ubiquitin protein ligase | | | | | | | |
| 133 | 11194 | ABCB8 | ATP-binding cassette, | x | | | | x | | |
| | | (SEQ ID | sub-family B | | | | | | | |
| | | NO: 133) | (MDR/TAP), member 8 | | | | | | | |
| 134 | 8878 | SQSTM1 | sequestosome 1 | | | | | x | | |
| | | (SEQ ID | | | | | | | | |
| | | NO: 134) | | | | | | | | |
| 135 | 30848 | CTAG2 | cancer/testis antigen 2 | x | | | | | | |
| | | (SEQ ID | | | | | | | | |
| | | NO: 135) | | | | | | | | |

The GeneID and Gene Symbol can be found on the NCBI website available at www.ncbi.nlm.nih.gov. More information about the gene can be found by accessing the NCBI website and entering the GeneID or Gene Symbol, for instance.

### Example 8

### Identification of the Pre-Treatment Autoantibody Response in Melanoma Patients

The pre-treatment (T0 or baseline) autoantibody response of melanoma patients has the potential to predict clinical response or longer survival of melanoma patients. Serum samples from 193 melanoma patients were obtained before starting treatment with anti-CTLA-4 (ipilimumab), anti-PD-1 (nivolumab or pembrolizumab) or anti-CTLA-4/anti-PD-1 combination therapy. The autoantibody levels of serum samples from melanoma patients were compared with autoantibody profiles of 148 healthy volunteer samples using based statistical technique Significance of microarrays (SAM). A positive SAM score-d and fold-change greater than 1 indicates that the autoantibody is elevated in the melanoma group compared to the control group. A negative SAM score-d and fold-change less than 1 indicates that the autoantibody level is lower in the melanoma group compared to the control group.

The preexisting autoantibody repertoire of metastatic melanoma patients at baseline is shown in Table 2. Autoantibody targets in table 2 are top-down ranked by their calculated SAM Score d. The correlation of baseline autoantibodies with different clinical endpoints such as the occurrence of irAEs or clinical response (disease control rate, DCR) is shown in separate tables (T).

Table 2 shows 36 autoantibody targets with higher reactivity in the melanoma group compared to healthy controls, which is indicated by a positive fold change: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, and NOVA2.

There were also 11 autoantibodies with lower reactivity (negative fold-change) in the melanoma group compared to healthy control samples: SNRPA, NRIP1, UBAP1, TEX264, PLIN2, LAMC1, CENPH, USB1, ABCB8, C15orf48/NMES1, and MAGED1.

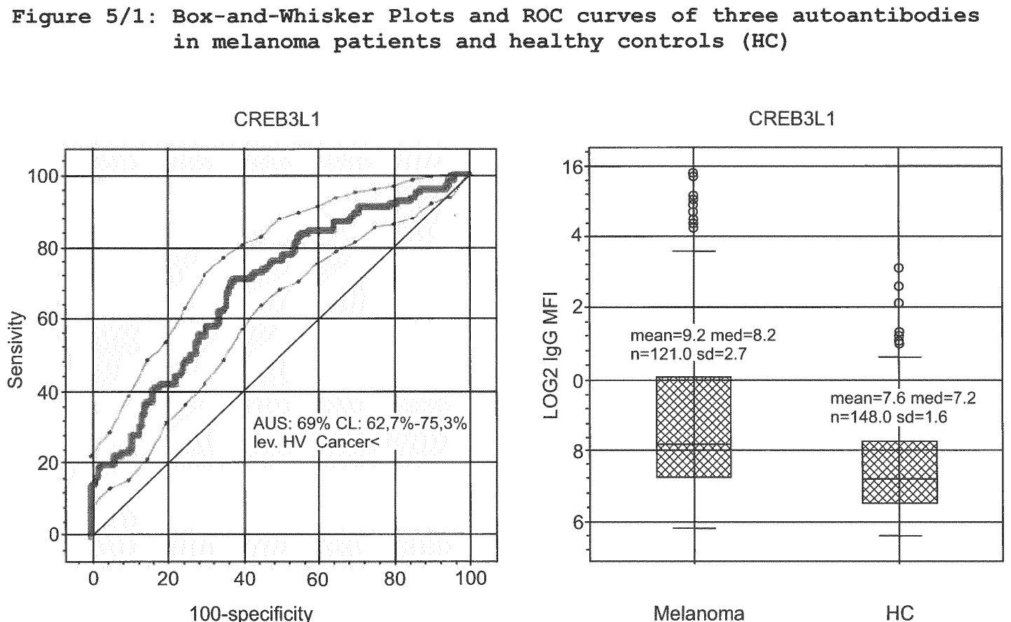

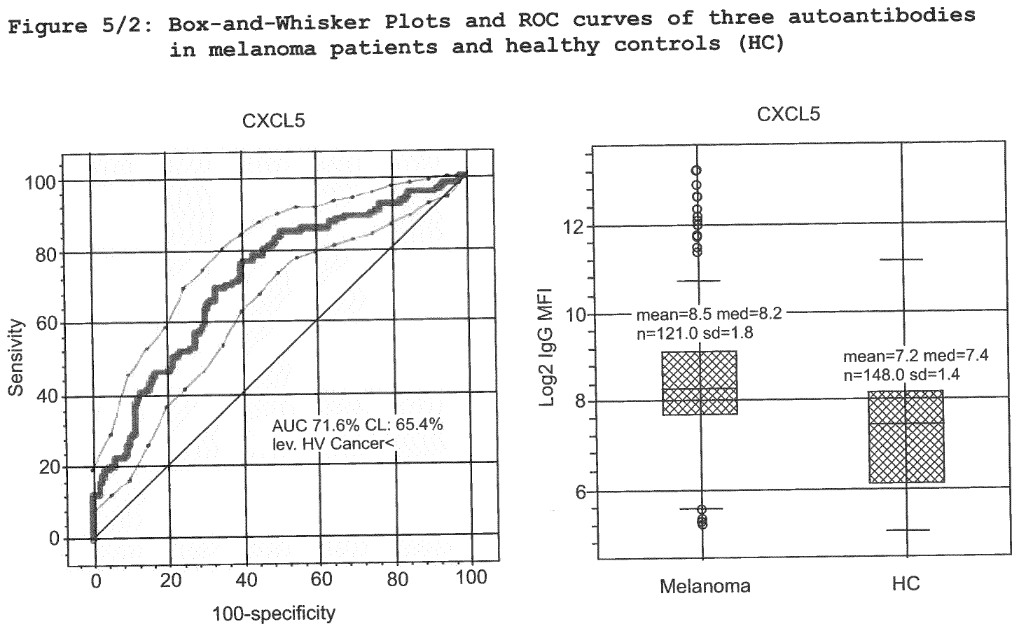

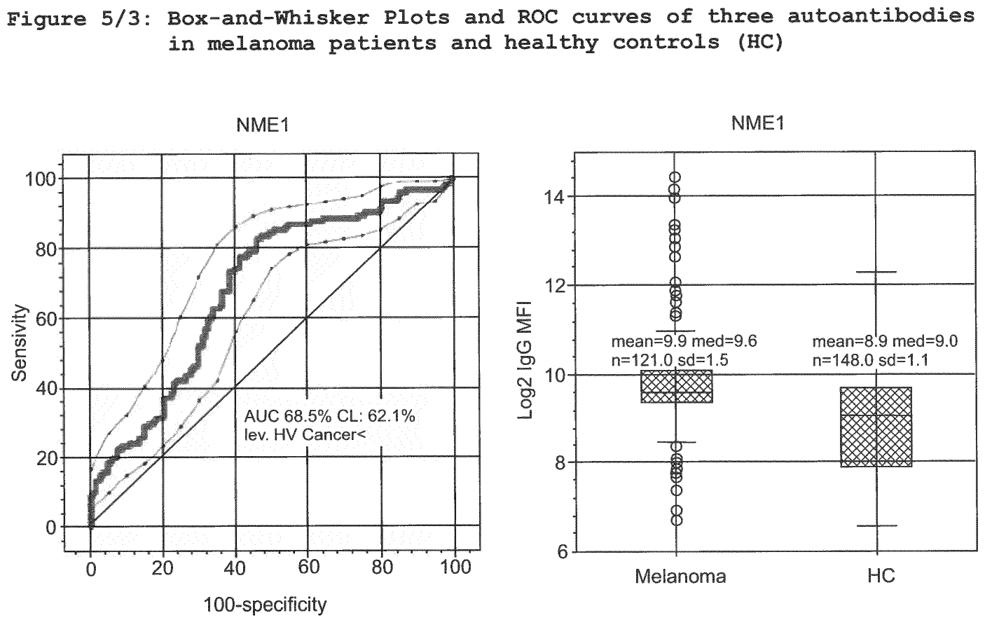

FIG. 5 shows Box-and-Whisker plots and ROC curves of three autoantibodies: CREB3L1; CXCL5; and NME1, with higher reactivity in serum samples of melanoma patients compared to healthy controls. The calculated area under the curve (AUC) of CREB3L1, CXCL5, and NME1 is 69%, 72%, and 69%, respectively.

CREB3L1 is also referred to as “Cyclic AMP-responsive element-binding protein 3-like protein 1”, “Old astrocyte specifically-induced substance”, and OASIS. CREB3L1 is a transcription factor that represses expression of genes regulating metastasis, invasion, and angiogenesis. Baseline anti-CREB3L1 antibodies also predict the development of irAEs following treatment with different checkpoint inhibitors (Table 4) including ipilimumab (Table 6).

CXCL5 is also referred to as “C-X-C motif chemokine 5”, “Epithelial-derived neutrophil-activating protein 78”, “Neutrophil-activating peptide ENA-78”, “Small-inducible cytokine B5”, and ENA78. CXCL5 is a chemokine, which stimulates the chemotaxis of neutrophils possessing angiogenic properties following binding the binds to cell surface chemokine receptor CXCR2. Tumor-associated neutrophils are increasingly recognized for their ability to promote tumor progression, mediate resistance to therapy, and regulate immunosuppression via the CXCL5/CXCR2 axis.

NME1 is also referred to as “Nucleoside diphosphate kinase A (EC:2.7.4.6)”, “NDP kinase A”, “Granzyme A-activated DNase”, “Metastasis inhibition factor nm23”, “Tumor metastatic process-associated protein”, GAAD, NM23-H1, NME1, NDPKA, and. NM23. Expression of the metastasis suppressor NME1 in melanoma is associated with reduced cellular motility and invasion in vitro and metastasis.

The three examples demonstrate that the autoantibody response of tumor patients is directed against a diverse set of proteins, which play a role in cancer processes.

TABLE 2
Autoantibody profile of melanoma patients

| | | | SAM Score | SAM fold-change | | | | | | |
|---|---|---|---|---|---|---|---|---|---|---|
| Marker | Gene | Gene | d HC vs | HC vs | | | | | | |
| No | ID | Symbol | Melanoma | Melanoma | T3 | T4 | T5 | T6 | T7 | T8 |
| 61 | 6181 | RPLP2 | 6.59 | 3.08 | x | | | | | |
| 11 | 1485 | CTAG1B | 5.70 | 3.70 | x | x | | | | |
| 108 | 1938 | EEF2 | 5.40 | 2.51 | x | | | | | |
| 105 | 6374 | CXCL5 | 5.40 | 2.45 | x | | | | | |
| 106 | 22826 | DNAJC8 | 5.04 | 2.26 | x | | | | | |
| 3 | 90993 | CREB3L1 | 4.94 | 2.90 | x | | x | | x | |
| 98 | 10000 | AKT3 | 4.86 | 1.77 | x | | | | | |
| 104 | 10563 | CXCL13 | 4.86 | 1.88 | x | | | | | |
| 103 | 4830 | NME1 | 4.73 | 1.98 | x | | | | | |
| 99 | 307 | ANXA4 | 4.53 | 1.73 | x | | | | | |
| 30 | 11214 | AKAP13 | 4.50 | 1.96 | x | | | | | |
| 102 | 30850 | CDR2L | 4.48 | 2.19 | x | | | | | |
| 100 | 483 | ATP1B3 | 4.46 | 1.71 | x | | | | | |
| 107 | 1845 | DUSP3 | 4.38 | 1.92 | x | | | | | |
| 62 | 6382 | SDC1 | 4.10 | 1.47 | x | | | | | |
| 96 | 29894 | CPSF1 | 4.09 | 1.83 | x | | | | | |
| 97 | 156 | GRK2 | 4.04 | 2.17 | x | | | | | |
| 63 | 6434 | TRA2B | 4.04 | 1.36 | x | | | | | |
| 101 | 613 | BCR | 4.01 | 1.57 | x | | | | | |
| 13 | 1457 | CSNK2A1 | 4.01 | 1.87 | x | | | | | x |
| 74 | 408 | ARRB1 | 3.91 | 1.80 | x | | | | | x |
| 88 | 2870 | GRK6 | 3.68 | 1.43 | x | x | | | | |
| 135 | 30848 | CTAG2 | 3.54 | 2.04 | x | | | | | |
| 10 | 4282 | MIF | 2.08 | 1.26 | x | x | | | | |
| 27 | 2065 | ERBB3 | 1.95 | 1.23 | x | | x | | | |
| 128 | 51684 | SUFU | 1.92 | 1.27 | x | | | | | |
| 119 | 8945 | BTRC | 1.90 | 1.33 | x | | | | | |
| 126 | 59307 | SIGIRR | 1.87 | 1.39 | x | | | | | |
| 115 | 26037 | SIPA1L1 | 1.83 | 1.34 | x | | | | x | |
| 72 | 60 | ACTB | 1.75 | 1.31 | x | | | | | x |
| 73 | 4302 | MLLT6 | 1.75 | 1.31 | x | | | | | x |
| 86 | 6464 | SHC1 | 1.70 | 1.20 | x | | | | | x |
| 12 | 10486 | CAP2 | 1.64 | 1.23 | x | | x | | | |
| 15 | 10243 | GPHN | 1.63 | 1.19 | x | x | | x | | |
| 60 | 361 | AQP4 | 1.62 | 1.24 | x | x | | | | |
| 8 | 4858 | NOVA2 | 1.52 | 1.42 | x | | | | | x |
| 56 | 6626 | SNRPA | −1.66 | 0.75 | x | | | | | x |
| 50 | 8204 | NRIP1 | −1.89 | 0.72 | x | | | | | x |
| 118 | 51271 | UBAP1 | −1.90 | 0.72 | x | | | | x | |
| 2 | 51368 | TEX264 | −2.19 | 0.65 | x | | x | | | x |
| 131 | 123 | PLIN2 | −2.20 | 0.65 | x | | | | x | |
| 78 | 3915 | LAMC1 | −2.25 | 0.64 | x | | | | | x |
| 40 | 64946 | CENPH | −2.25 | 0.70 | x | | | | x | |
| 37 | 79650 | USB1 | −2.56 | 0.73 | x | | | x | | |
| 133 | 11194 | ABCB8 | −2.61 | 0.77 | x | | | | x | |
| 9 | 84419 | C15orf48; | −2.66 | 0.74 | x | x | | | | x |
| | | NMES1 | | | | | | | | |
| 109 | 9500 | MAGED1 | −4.28 | 0.65 | x | | | | | |

### Example 9

### Identification of Autoantibodies Associated or Predicting Survival and Clinical Response to Immune-Oncology Agents

The role of B cells and their secreted products in driving anti-cancer immunity is only insufficiently understood. Autoantibodies produced by B cells may have both pro- and anti-tumor effects. Thus, autoantibodies may serve as biomarkers of the general immune fitness of a cancer patient and his ability to respond to immune-oncology agents.

The autoantibody reactivity of serum samples from 193 melanoma patients treated with anti-CTLA-4 (ipilimumab), anti-PD-1 (nivolumab or pembrolizumab) or anti-CTLA-4/anti-PD-1 combination therapy was analyzed. To evaluate the difference in autoantibody levels between the clinical outcomes DCR and PD, the statistical test SAM was applied.

A positive SAM score-d and fold-change greater than 1 indicates that the autoantibody is elevated in the melanoma group achieving DCR compared to patients who have had PD. A negative SAM score-d and fold-change less than 1 indicates that the autoantibody levels are decreased in the melanoma group achieving DCR compared to patients who have had PD.

Spearman's rank correlation analysis was used to evaluate the association between autoantibody levels and overall survival (OS).

Ten autoantibodies predicted a clinical response referred to as “disease control rate” (DCR) to immune-oncology treatments in general.

Six baseline autoantibodies directed towards SIVA1, IGF2BP2, AQP4, C15orf48, GPHN, and CTAG1B appear to be predictors of DCR.

The level of four baseline autoantibodies directed towards GRK6, FGFR1, MIF, and GRAMD4 were higher in the group of patients who have had PD compared to patients achieving DCR:

These autoantibodies appear to be predictors of non-response or PD to immune-oncology treatment in general.

Higher baseline anti-PAPOLG antibodies were weakly associated with overall survival (Spearman's rank correlation coefficient r=0.32).

Table 3 shows autoantibodies associated with OS and DCR in melanoma patients treated with different checkpoint inhibitors.

TABLE 3
Autoantibodies associated OS and DCR in melanoma patients

| | | | Spearman's | SAM | SAM |
|---|---|---|---|---|---|
| | | | R-value | Score d | fold-change |
| ID | Gene ID | Gene Symbol | OS | DCR at T0 | DCR at T0 |
| 59 | 10572 | SIVA1 | 0.02 | 2.12 | 1.66 |
| 14 | 10644 | IGF2BP2 | 0.07 | 1.99 | 1.65 |
| 60 | 361 | AQP4 | −0.15 | 1.85 | 1.49 |
| 9 | 84419 | C15orf48 | 0.11 | 1.85 | 1.46 |
| 15 | 10243 | GPHN | 0.09 | 1.84 | 1.40 |
| 11 | 1485 | CTAG1B | 0.08 | 1.83 | 2.21 |
| 21 | 64895 | PAPOLG | 0.32 | 0.24 | 1.05 |
| 88 | 2870 | GRK6 | −0.12 | −1.80 | 0.71 |
| 90 | 2260 | FGFR1 | −0.07 | −1.86 | 0.67 |
| 10 | 4282 | MIF | −0.07 | −1.88 | 0.67 |
| 1 | 23151 | GRAMD4 | −0.18 | −1.92 | 0.68 |

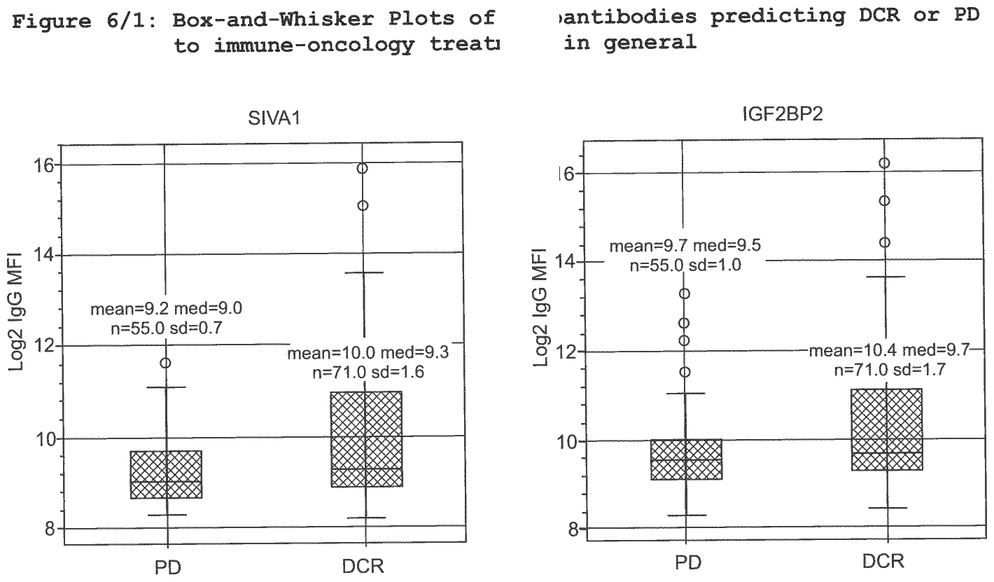

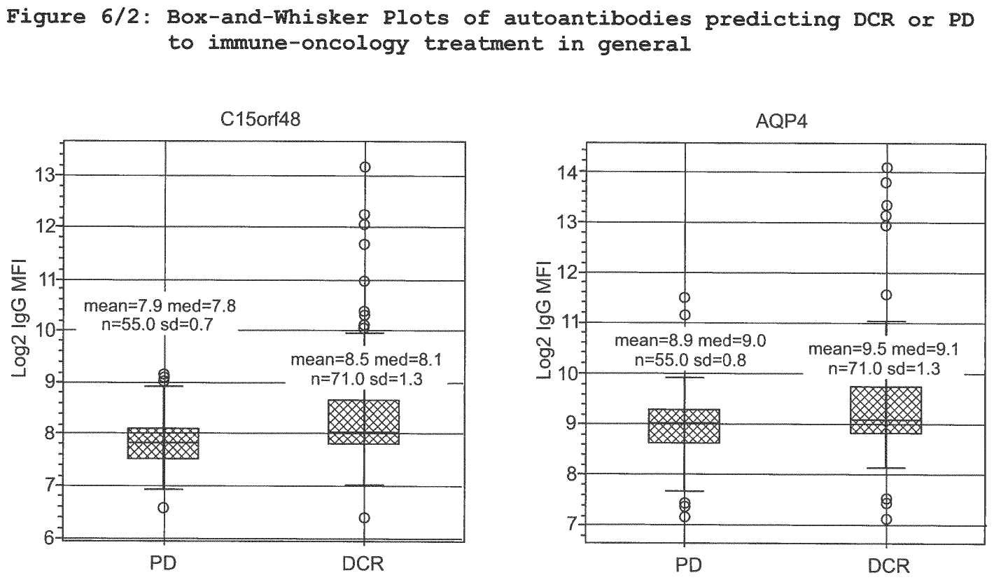

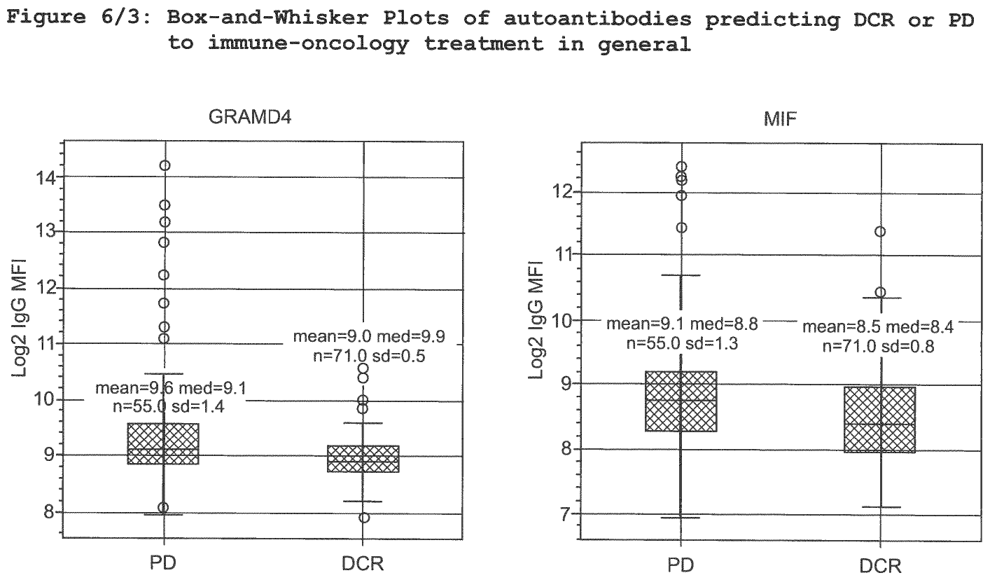

FIG. 6 shows four baseline autoantibodies, SIVA1, IGF2BP2, AQP4, and C15orf48, which predict DCR and two baseline autoantibodies, MIF and GRAMD4, which predict PD to checkpoint inhibitor treatment in general.

SIVA1 is also referred to as “Apoptosis regulatory protein Siva”, “CD27-binding protein”, CD27BP, or SIVA1. SIVA1 plays an important role in the apoptotic (programmed cell death) pathway induced by the CD27 antigen, a member of the tumor necrosis factor receptor (TFNR) superfamily. Higher baseline anti-SIVA1 antibodies were found in patients who achieve DCR compared to patients who have had PD following checkpoint inhibitor treatment. Furthermore, higher anti-SIVA1 antibodies were also found in melanoma patients who achieve DCR compared to patients who have had PD following treatment with the PD-1/PD-L1 pathway blocker pembrolizumab (Table 7).

IGF2BP2 is also referred to as “Insulin-like growth factor 2 mRNA-binding protein 2”, “Hepatocellular carcinoma autoantigen p62”, “IGF-II mRNA-binding protein 2”, “VICKZ family member 2”, IGF2BP2, IMP2, or VICKZ2. The gene encoding IGF2BP2 is amplified and overexpressed in many human cancers, accompanied by a poorer prognosis (Dai et al., 2017).

Higher baseline anti-IGF2BP2 antibodies were found in patients who achieve DCR compared to patients who have had PD following checkpoint inhibitor treatment. Furthermore, higher baseline anti-IGF2BP2 antibodies were also found in melanoma patients who achieve DCR compared to patients who have had PD following treatment with the PD-1/PD-L1 pathway blocker pembrolizumab (Table 7)

AQP4 is also referred to as “Aquaporin-4”, “Mercurial-insensitive water channel”, MIWC, or WCH4. AQP4 is a water channel protein, predominantly found in tissues of neuronal origin. Anti-AQP4 antibodies are found in the autoimmune disorder, neuromyelitis optica, NMO, which affects the optics nerves and spinal cord of individuals. Higher baseline anti-AQP4 antibodies were found in patients who achieve DCR compared to patients who have had PD following checkpoint inhibitor treatment. Furthermore, higher levels of anti-AQP4 antibodies were found in melanoma patients compared to healthy controls (Table 2).

C15orf48 is also referred to as “normal mucosa of esophagus-specific gene 1 protein”, Protein FOAP-11, MIR147BHG, or NMES1. Higher baseline anti-C15orf48 antibody levels were found in patients who achieve DCR compared to patients who have had PD following checkpoint inhibitor treatment. Furthermore, higher autoantibody levels were also found in melanoma patients who achieve DCR compared to patients who have had PD treatment with the PD-1/PD-L1 pathway blocker pembrolizumab (Table 7).

GRAMD4 is also referred to as “GRAM domain-containing protein 4”, “Death-inducing protein”, DIP, or KIAA0767. GRAMD4 has been reported as a pro-apoptotic protein. Higher baseline levels of anti-GRAMD4 antibodies were found in patients who have had PD compared to patients who achieved DCR following checkpoint inhibitor treatment. Higher anti-GRAMD3 antibodies were also associated with PD, shorter PFS and shorter survival in melanoma patients treated with the CTLA-4 inhibitor ipilimumab (Table 5).

MIF is also referred to as “Macrophage migration inhibitory factor (EC:5.3.2.1)”, “Glycosylation-inhibiting factor”, “L-dopachrome tautomerase (EC:5.3.3.12)”, or GIF. MIF is a pro-inflammatory cytokine, which is overexpressed in malignant melanoma. Higher baseline levels of anti-MIF antibodies were found in patients who have had PD compared to patients who achieved DCR following checkpoint inhibitor treatment. Furthermore, higher baseline levels of anti-MIF antibodies were found in melanoma patients compared to healthy controls (Table 2) and in melanoma patients who do not develop irAEs compared to patients who developed irAE after treatment with the PD-1/PD-L1 pathway blocker pembrolizumab (Table 8).

### Example 10

### Identification of Baseline Autoantibodies Predicting irAE in Melanoma Patients Following Treatment with Different Checkpoint Inhibitors

Despite important clinical benefits, checkpoint inhibitors are associated with immune-related adverse events (irAEs). The mechanisms by which checkpoint inhibitors induce irAEs are not completely understood. It is believed that by blocking negative checkpoints a general immunologic enhancement occurs. It is also possible that by unleashing the immune-checkpoints that control tolerance, autoreactive lymphocytes are activated, which could be either T cells or B cells. It is well known that in autoimmune diseases autoreactive B cells produce autoantibodies that can induce tissue damage via ADCC. Thus, epitope spreading towards self-antigens may be an indicator for irAEs.

Autoantibodies predicting irAEs were identified in pre-treatment samples from patients receiving different checkpoint inhibitors such as anti-CTLA-4, anti-PD-1 or combination therapies of anti-CTLA-4 and anti-PD-1. To evaluate the difference in autoantibody levels between patients experiencing an irAE and those who do not, the statistical test SAM was applied.

A positive SAM score-d and fold-change greater than 1 indicates that the autoantibody is elevated in the melanoma group who have had an irAE compared to those without an irAE. A negative SAM score-d and fold-change less than 1 indicates that the autoantibody levels are lower in the melanoma group who have had an irAE compared those without an irAE.

Table 4 includes 12 autoantibodies reacting with TEX264, CREB3L1, HSPA1B, SPTB, MUC12, ERBB3, ATG4D, CASP10, FOXO1, FRS2, and PPP1R12A, which appear to predict irAEs in baseline samples. Table 4 includes five autoantibodies, HSPA2, SMAD9, HIST2H2AA3, S100A8, and SDCBP, which predict that patients having higher autoantibody levels do not develop an irAE.

TABLE 4
Baseline autoantibodies predicting irAE in melanoma patients
following treatment with different checkpoint inhibitors

| | | | SAM Score.d. | SAM Fold.Change |
|---|---|---|---|---|
| ID | Gene ID | Gene Symbol | irAE at T0 | irAE at T0 |
| 2 | 51368 | TEX264 | 2.41 | 1.93 |
| 3 | 90993 | CREB3L1 | 2.33 | 2.42 |
| 17 | 3304 | HSPA1B | 2.17 | 1.63 |
| 18 | 6710 | SPTB | 2.17 | 1.63 |
| 57 | 10071 | MUC12 | 2.06 | 1.49 |
| 27 | 2065 | ERBB3 | 2.04 | 1.36 |
| 28 | 84971 | ATG4D | 2.03 | 1.36 |
| 94 | 843 | CASP10 | 2.02 | 1.36 |
| 95 | 2308 | FOXO1 | 1.99 | 1.83 |
| 5 | 10818 | FRS2 | 1.92 | 1.72 |
| 93 | 4659 | PPP1R12A | 1.90 | 1.59 |
| 12 | 10486 | CAP2 | 1.87 | 1.43 |
| 31 | 3306 | HSPA2 | −1.85 | 0.72 |
| 32 | 4093 | SMAD9 | −1.96 | 0.71 |
| 58 | 8337 | HIST2H2AA3 | −2.04 | 0.73 |
| 6 | 6279 | S100A8 | −2.15 | 0.76 |
| 16 | 6386 | SDCBP | −2.73 | 0.60 |

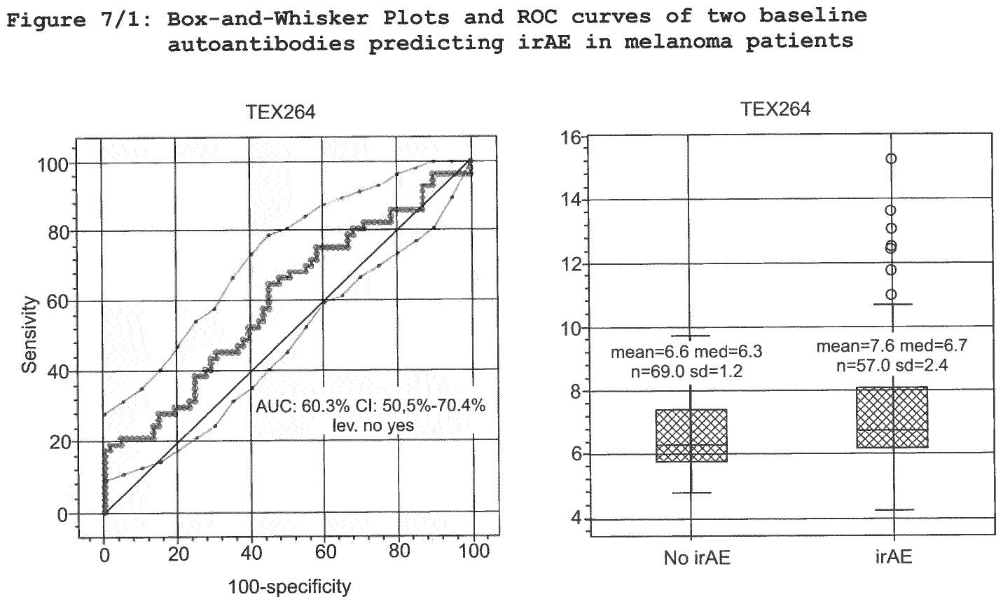

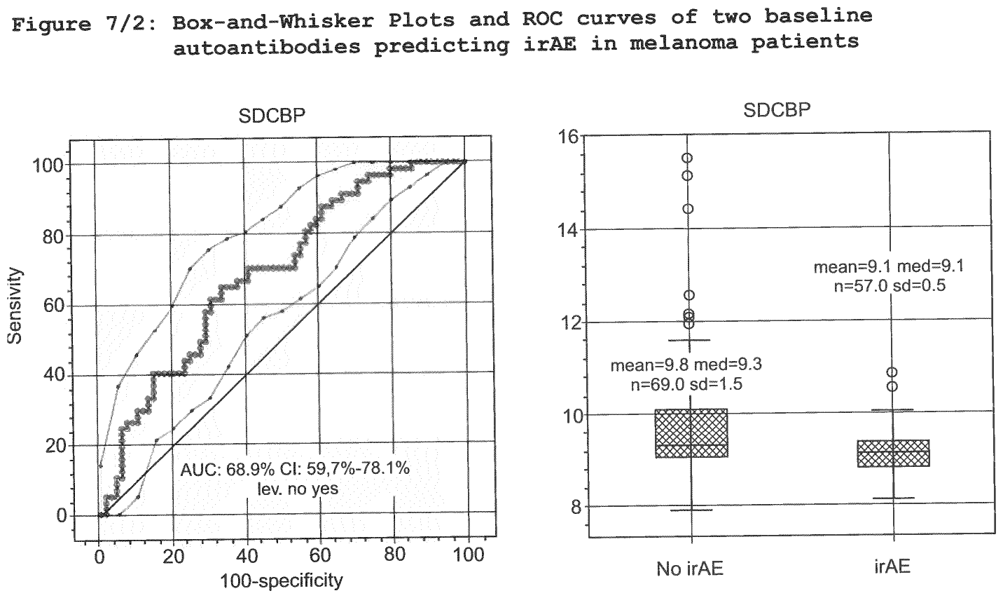

FIG. 7 shows Box-and-Whisker Plots and ROC curves of baseline levels of anti-TEX264 and anti-SDCBP antibodies that allow to discriminate patients developing irAE from those who do not develop irAE in response to checkpoint inhibitor treatment. The calculated area under the curve (AUC) of anti-TEX264 and anti-SDCBP is 60% and 69%, respectively.

TEX264 is also referred to as “Testis-expressed protein 264”, or “Putative secreted protein Zsig11”. The function of the gene encoding TEX264 is currently unknown. Elevated baseline anti-TEX264 antibodies predict the development of irAE to checkpoint inhibitors. Furthermore, anti-TEX264 antibodies also predict clinical response as defined as DCR (Table 7) and the development of irAEs in patients treated with the anti-PD-1 blocker pembrolizumab (Table 8).

SDCBP is also referred to as “syntenin-1”, “Melanoma differentiation-associated protein 9”, MDA-9, “Pro-TGF-alpha cytoplasmic domain-interacting protein 18”, TACIP18, “Scaffold protein Pbp1”, “Syndecan-binding protein 1”, MDA9, or SYCL. SDCBP is expressed in melanoma and influences metastasis by regulating both tumor cells and the microenvironment (Das et al., 2012).

Higher baseline anti-SDCBP antibodies were found in patients who do not develop irAEs following immune checkpoint inhibitor treatment. Furthermore, anti-SDCBP antibody levels were higher in patients who do not develop irAEs following treatment with anti-CTLA-4 inhibitor ipilimumab (Table 6).

### Example 11

### Identification of Autoantibodies Associated or Predicting Survival and Clinical Response to Ipilimumab Treatment

One of the reasons to terminate a patient's cancer therapy or to change the therapy is disease progression.

To identify autoantibodies that allow one to identify patients who benefit from ipilimumab therapy, serum samples from 82 melanoma patients treated with ipilimumab were analyzed.

Biomarkers correlating with progression-free survival (PFS) or overall survival (OS) were calculated using Spearman's correlation. To evaluate the difference in autoantibody levels between the clinical outcomes DCR and PD, the statistical test SAM was applied.

A positive SAM score-d and fold-change greater than 1 indicates that the autoantibody is elevated in the melanoma group who achieved DCR compared to patients who have had PD. A negative SAM score-d and fold-change less than 1 indicates that the autoantibody levels are lower in the melanoma group achieving DCR compared those who have had PD.

Table 5 shows 13 autoantibodies, FRS2, GPHN, BIRC5, EIF3E, CENPH, PAPOLG, HUS1, GNG12, CCDC51, USB1, GRAMD4, RPS6KA1, and BCL7B, correlating positively or negatively with PFS, OS, or predict DCR or PD in baseline samples.

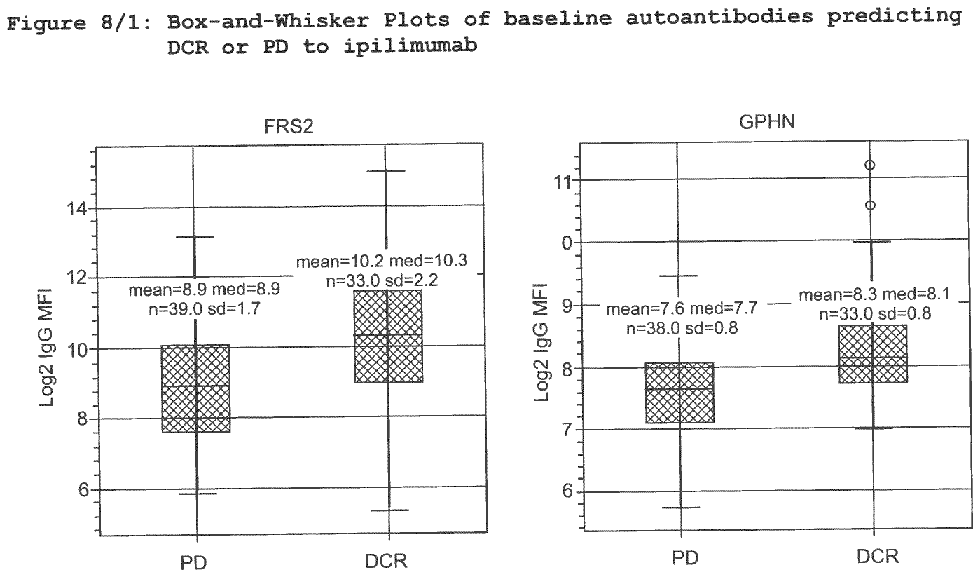

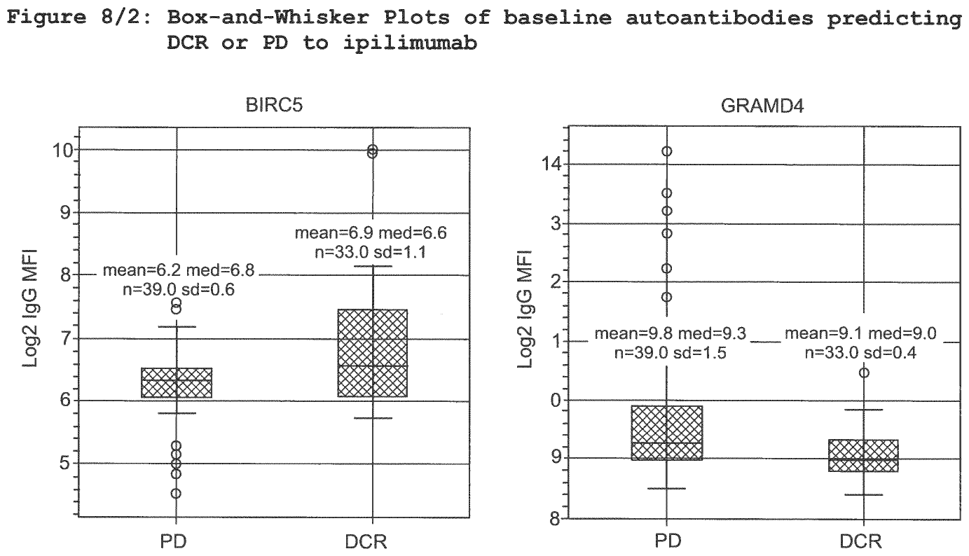

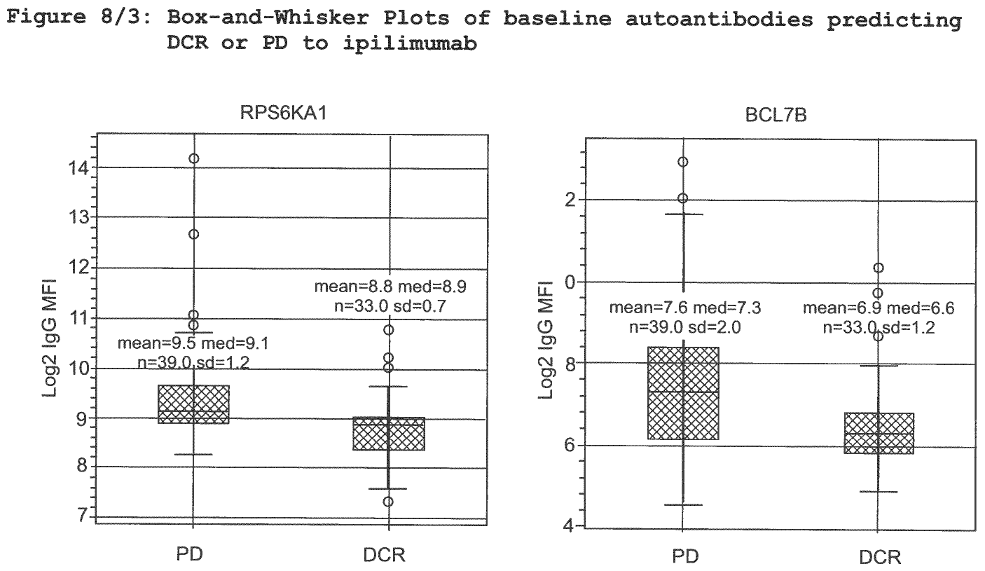

FIG. 8 shows Box-and-Whisker plots of six baseline autoantibodies, FRS2, GPHN, BIRC5, GRAMD4, RPS6Ka2, and BCL7B, predicting DCR or PD to ipilimumab.

BIRC5 is also known as “Baculoviral IAP repeat-containing protein 5”, “Apoptosis inhibitor 4”, “Apoptosis inhibitor surviving”API4, or IAP4. BIRC5 is overexpressed in human cancer and plays a role in inhibition of apoptosis, resistance to chemotherapy and aggressiveness of tumors (Garg et al., 2016). Higher baseline anti-BIRC5 antibody levels were found in patients who achieve DCR compared to patients with PD following ipilimumab treatment.

FRS2 is also known as “Fibroblast growth factor receptor substrate 2”, “FGFR-signaling adaptor SNT”, “Suc1-associated neurotrophic factor target 1”, or SNT-1. FRS2 is overexpressed and amplified in several cancer types. It serves as a docking protein for receptor tyrosine kinases, which mediate proliferation, survival, migration, and differentiation (Luo and Hahn, 2015).

Higher baseline anti-FRS2 antibody levels were found in patients who achieve DCR compared to patients with PD following ipilimumab treatment. Furthermore, higher baseline levels of anti-FRS2 antibodies also predict both response to anti-CTLA-4 treatment (Table 5) and the development of irAE (Table 6).

BCL7B also known as §B-cell CLL/lymphoma 7 protein family member B″ is a member of the BCL7 gene family, which is involved in the modulation of multiple pathways, including Wnt and apoptosis. The BCL7 family is involved in cancer incidence, progression, and development (Uehara et al., 2015). Higher baseline anti-BCL7B antibody levels were found in patients who have had PD compared to patients who achieve DCR following ipilimumab treatment.

RPS6KA1 is also known as “Ribosomal protein S6 kinase alpha-1 (EC:2.7.11.1)”, “MAP kinase-activated protein kinase 1a”, p90RSK1, RSK-1, or MAPKAPK1A. The RSK (90 kDa ribosomal S6 kinase) family comprises a group of highly related serine/threonine kinases that regulate diverse cellular processes, including cell growth, proliferation, survival and motility. Dysregulated RSK expression and activity has been associated with multiple cancer types (Houles and Roux, 2017).

Higher baseline anti-RPS6KA1 antibody levels were found in patients who have had PD compared to patients who achieve DCR following ipilimumab treatment.

GPHN is also known as “Gephyrin”, “Molybdopterin adenylyltransferase (EC:2.7.7.75)”, MPT, or KIAA1385. Gephyrin is a 93 kDa multi-functional protein that is a component of the postsynaptic protein network of inhibitory synapses. In non-neuronal tissues, the encoded protein is also required for molybdenum cofactor biosynthesis, a cofactor of sulfite oxidase, aldehyde oxidase, and xanthine oxidoreductase (Smolinsky et al., 2008). Higher baseline anti.GPHN antibody levels were found in patients who achieve DCR compared to patients with PD following ipilimumab treatment. Besides predicting response to anti-CTLA-4 therapy, GPHN is also a useful marker to discriminate melanoma patients from normal humans (Table 2) and predicts DCR in melanoma patients treated with different checkpoint inhibitors (Table 3).

TABLE 5
Autoantibodies associated with PFS, OS and DCR
in melanoma patients treated with ipilimumab

| | | | | | SAM | SAMR |
|---|---|---|---|---|---|---|
| | | Gene | R-value | R-value | Score.d. | Fold.Change |
| ID | Gene ID | Symbol | PFS | OS | DCR at T0 | DCR at T0 |
| 5 | 10818 | FRS2 | 0.21 | 0.2 | 2.23 | 2.55 |
| 15 | 10243 | GPHN | 0.16 | 0.24 | 2.18 | 1.68 |
| 33 | 332 | BIRC5 | 0.05 | 0.06 | 1.8 | 1.54 |
| 39 | 3646 | EIF3E | 0.08 | 0.33 | 1.09 | 1.31 |
| 40 | 64946 | CENPH | 0.18 | 0.31 | 0.88 | 1.31 |
| 21 | 64895 | PAPOLG | 0.28 | 0.37 | 0.53 | 1.14 |
| 43 | 3364 | HUS1 | 0.34 | 0.16 | −0.01 | 1 |
| 41 | 55970 | GNG12 | 0.32 | 0.24 | −0.21 | 0.95 |
| 42 | 79714 | CCDC51 | −0.32 | −0.15 | −0.34 | 0.88 |
| 37 | 79650 | USB1 | −0.16 | −0.06 | −1.81 | 0.6 |
| 1 | 23151 | GRAMD4 | −0.3 | −0.32 | −1.92 | 0.58 |
| 36 | 6195 | RPS6KA1 | −0.2 | −0.17 | −1.92 | 0.61 |
| 38 | 9275 | BCL7B | −0.1 | −0.17 | −1.95 | 0.48 |

### Example 12

### Identification of Autoantibodies Associated with irAEs in Patients Treated with Ipilimumab

Autoantibodies predicting irAEs were identified in pre-treatment samples from patients receiving anti-CTLA-4 therapy. To evaluate the difference in autoantibody levels between patients experiencing an irAE and those who do not, the statistical test SAM was applied.

Table 6 includes 13 autoantibodies reacting with EOMES, CREB3L1, FRS2, PLIN2, SIPA1L1, ABCB8, MAPT, ATG4D, XRCC5, XRCC6, UBAP1, TRIP4, and EIF4E2, which appear to predict irAEs in baseline samples.

A positive SAM score-d and fold-change greater than 1 indicates that the autoantibody is elevated in the melanoma group who have had an irAE compared to those without an irAE. A negative SAM score-d and fold-change less than 1 indicates that the autoantibody levels are lower in the melanoma group who have had an irAE compared those without an irAE.

Table 6 includes eight autoantibodies, POLR3B, ELMO2, SUMO2, RFWD2, SQSTM1, SDCBP, HSPD1, and IL17A, which predict that patients having higher autoantibody levels do not develop an irAE.

TABLE 6
Baseline autoantibodies predicting irAEs in melanoma
patients treated with ipilimumab

| | | Gene | SAM Score.d. | SAM Fold.Change |
|---|---|---|---|---|
| ID | Gene ID | Symbol | irAE at T0 | irAE at T0 |
| 26 | 8320 | EOMES | 2.30 | 2.82 |
| 3 | 90993 | CREB3L1 | 2.28 | 2.60 |
| 5 | 10818 | FRS2 | 2.11 | 2.28 |
| 131 | 123 | PLIN2 | 2.11 | 2.13 |
| 115 | 26037 | SIPA1L1 | 1.96 | 1.85 |
| 133 | 11194 | ABCB8 | 1.93 | 1.47 |
| 113 | 4137 | MAPT | 1.87 | 1.58 |
| 28 | 84971 | ATG4D | 1.83 | 1.34 |
| 24 | 7520 | XRCC5 | 1.82 | 1.55 |
| 25 | 2547 | XRCC6 | 1.82 | 1.55 |
| 118 | 51271 | UBAP1 | 1.81 | 1.75 |
| 117 | 9325 | TRIP4 | 1.81 | 1.57 |
| 110 | 9470 | EIF4E2 | 1.81 | 1.62 |
| 114 | 55703 | POLR3B | −1.84 | 0.55 |
| 29 | 63916 | ELMO2 | −1.89 | 0.55 |
| 116 | 6613 | SUMO2 | −1.91 | 0.63 |
| 132 | 64326 | RFWD2 | −1.99 | 0.67 |
| 134 | 8878 | SQSTM1 | −2.02 | 0.69 |
| 16 | 6386 | SDCBP | −2.03 | 0.64 |
| 111 | 3329 | HSPD1 | −2.10 | 0.44 |
| 112 | 3605 | IL17A | −2.19 | 0.60 |

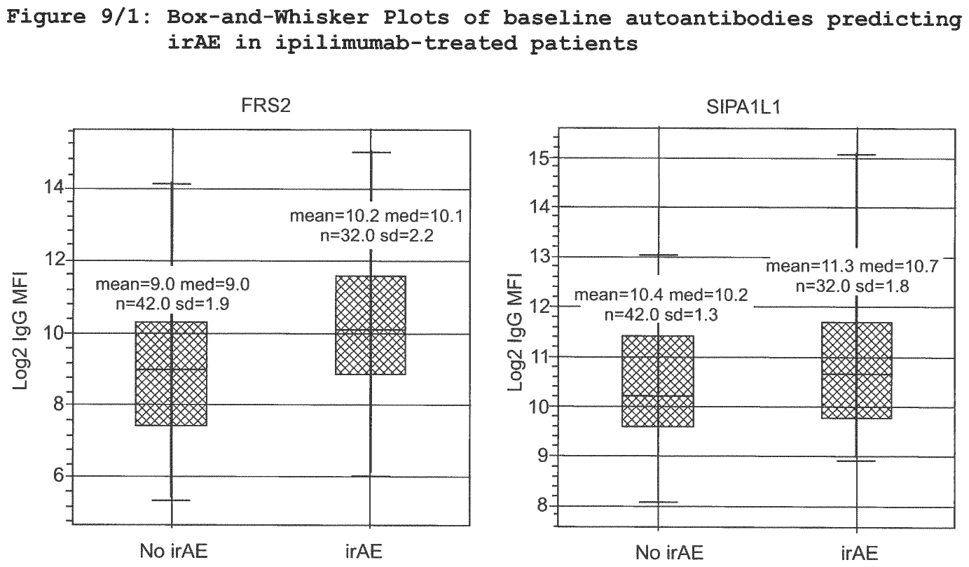

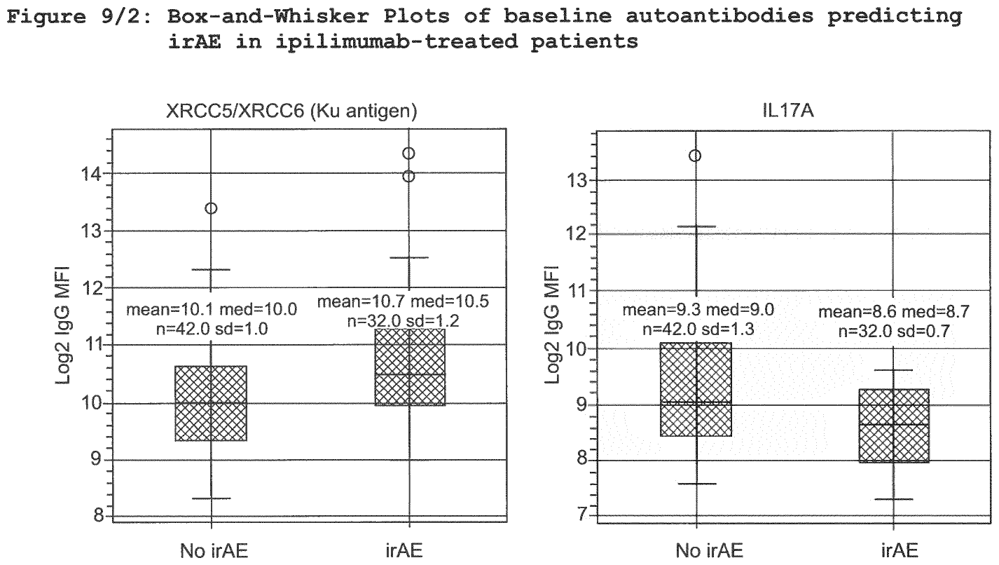

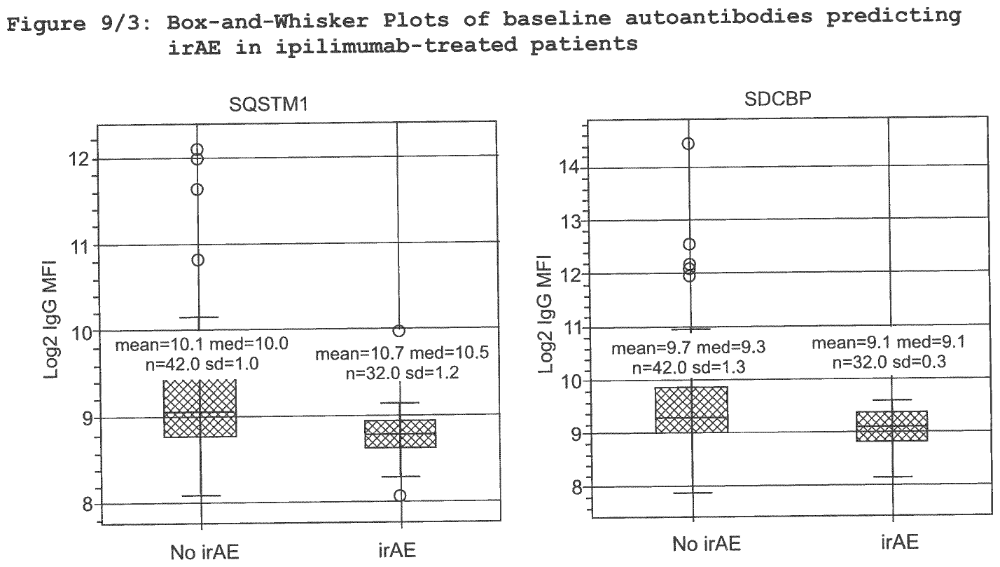

FIG. 9 shows Box-and-Whisker plots of six baseline autoantibodies, FRS2, SIPA1L1, XRCC5/XRCC6, IL17A, SQSTM1, and SDCBP, which are associated with the development of irAE in ipilimumab-treated patients.

Higher baseline levels of anti-FRS2 antibodies were found in patients who have had irAEs compared to those without irAEs following ipilimumab treatment. Furthermore, higher anti-FRS2 antibodies were found in patients achieving DCR compared to patients who have had PD following ipilimumab (Table 5).

SIPA1L1 is also known as “signal-induced proliferation-associated 1-like protein 1”, “High-risk human papilloma viruses E6 oncoproteins targeted protein 1”, E6TP1, or. KIAA0440. Besides predicting the development of irAEs, Higher baseline anti-SIPA1L1 antibodies were found in patients who have had irAEs compared to patients without irAEs following ipilimumab treatment.

Higher anti-SIPA1L1 were also found in melanoma patients compared to healthy controls (Table 2). Thus, anti-SIPA1L1 may be a useful marker to discriminate melanoma patients from normal humans.

A dimer of the antigens XRCC5 and XRCC6 form the Lupus Ku autoantigen protein. Higher baseline levels of autoantibodies to XRCC5/XRCC6 predict the development of irAE in ipilimumab treated patients. XRCC5 is also known as “X-ray repair cross-complementing protein 5”, Lupus Ku autoantigen protein p86, Ku80, or Ku86. XRCC6 is also known as “X-ray repair cross-complementing protein 6”, 70 kDa subunit of Ku antigen, Lupus Ku autoantigen protein p70, Ku70, or thyroid-lupus autoantigen.

Higher anti-XRCC5 and anti-XRCC6 antibody levels were detected in patients who have had irAEs compared to patients without irAEs following ipilimumab treatment.

Besides predicting the development of irAEs following anti-CTLA-4 therapy, anti-XRCC5/XRCC6 antibodies, also predict clinical response defined as DCR in melanoma patients treated with the PD-1/PD-L1 pathway blocker pembrolizumab (Table 7).

Higher levels of anti-IL17A antibodies are found in patients who do not develop irAEs compared to patients who had irAEs following ipilimumab treatment. IL17A is also known as “interleukin 17A”, CTLA8; or IL-17. IL17 and is a proinflammatory cytokine produced by activated T cells.

SQSTM1 is also known as “sequestosome 1”, p60, p62, A170, DMRV, OSIL, PDB3, ZIP3, p62B, NADGP, or FTDALS3. SQSTM1 is an autophagosome cargo protein that targets other proteins that bind to it for selective autophagy. It is also interacts with signaling molecules to promote the expression of inflammatory genes (Moscat et al., 2016). Higher anti-SQSTM1 antibodies are found in melanoma patients who do not develop irAEs compared to patients who had irAEs following ipilimumab treatment.

### Example 13

### Identification of Autoantibodies Associated or Predicting Survival and Clinical Response to Pembrolizumab Treatment

To identify autoantibodies that allow one to identify patients who benefit from treatment with PD-1/PD-L1 pathway inhibitors, serum samples from 41 melanoma patients treated with pembrolizumab were analyzed.

Biomarkers correlating with progression-free survival (PFS) or overall survival (OS) were calculated using Spearman's correlation. To evaluate the difference in autoantibody levels between the clinical outcomes DCR and PD, the statistical test SAM was applied.

A positive SAM score-d and fold-change greater than 1 indicates that the autoantibody is elevated in the melanoma group who achieved DCR compared to patients who have had PD. A negative SAM score-d and fold-change less than 1 indicates that the autoantibody levels are lower in the melanoma group achieving DCR compared those who have had PD.

Table 7 lists 42 autoantibody targets, which are associated with response or non-response to pembrolizumab therapy: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, FGA, CALR, GNAI2, IL36RN, S100A14, MMP3, SHC1, CSNK2A1, DFFA, LAMC1, S100A8, HDAC1, MSH2, CEACAM5, DHFR, and ARRB1.

Higher serum levels of ten autoantibodies were positively correlated with longer overall survival (OS, Spearman's correlation r>0.3): TRAF3IP3, C17orf85, HES1, CCNB1, SNRPD1, FGA, CALR, NRIP1, CSNK2A1, and SSB.

There were also four autoantibodies that were inversely correlated with overall survival and associated with shorter survival (Spearman's r<−0.3). SHC1, MMP3, GNAI2, and IL36RN.

Table 7 includes 19 baseline autoantibodies, which were elevated in patients, who achieve DCR following pembrolizumab treatment (SAM Score d>1.8): NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, and TEX264.

Furthermore, there were also an autoantibody signature comprising eight baseline autoantibodies that were elevated in patients with progressive disease (PD), who do not respond to pembrolizumab therapy (SAM DCR Score d<−1.8): ARRB1, DHFR, CEACAM5, MSH2, HDAC1, S100A8, LAMC1, and DFFA.

TABLE 7
Autoantibodies associated with PFS, OS and
DCR in melanoma patients treated with pembrolizumab

| | | | | | | | SAM |
|---|---|---|---|---|---|---|---|
| | | | | | | SAM | Fold- |
| | | | R- | R- | R- | Score d | change |
| | Gene | Gene | value | value | value | DCR | DCR at |
| ID | ID | Symbol | PFS | OS | DCR | at T0 | T0 |
| 8 | 4858 | NOVA2 | 0.40 | 0.24 | 0.35 | 2.79 | 4.48 |
| 26 | 8320 | EOMES | 0.31 | 0.15 | 0.38 | 2.31 | 4.81 |
| 23 | 6741 | SSB | 0.52 | 0.32 | 0.41 | 2.30 | 2.12 |
| 14 | 10644 | IGF2BP2 | 0.26 | 0.13 | 0.33 | 2.29 | 2.45 |
| 72 | 60 | ACTB | 0.27 | 0.09 | 0.32 | 2.19 | 2.28 |
| 73 | 4302 | MLLT6 | 0.27 | 0.09 | 0.32 | 2.19 | 2.28 |
| 22 | 6632 | SNRPD1 | 0.18 | 0.37 | 0.32 | 2.18 | 2.36 |
| 7 | 80342 | TRAF3IP3 | 0.49 | 0.41 | 0.42 | 2.18 | 1.67 |
| 4 | 55421 | C17orf85 | 0.38 | 0.38 | 0.29 | 2.12 | 2.46 |
| 19 | 3280 | HES1 | 0.26 | 0.37 | 0.33 | 1.96 | 2.25 |
| 76 | 2931 | GSK3A | 0.27 | 0.18 | 0.27 | 1.95 | 3.08 |
| 24 | 7520 | XRCC5 | 0.27 | 0.12 | 0.26 | 1.93 | 1.78 |
| 25 | 2547 | XRCC6 | 0.27 | 0.12 | 0.26 | 1.93 | 1.78 |
| 51 | 5504 | PPP1R2 | 0.23 | 0.16 | 0.32 | 1.93 | 2.33 |
| 9 | 84419 | C15orf48 | 0.24 | 0.18 | 0.34 | 1.91 | 1.58 |
| 81 | 5801 | PTPRR | 0.23 | 0.27 | 0.30 | 1.89 | 2.16 |
| 80 | 4150 | MAZ | 0.17 | 0.11 | 0.23 | 1.88 | 3.54 |
| 84 | 2316 | FLNA | 0.12 | −0.05 | 0.13 | 1.87 | 2.62 |
| 2 | 51368 | TEX264 | 0.37 | 0.23 | 0.25 | 1.87 | 2.66 |
| 55 | 6625 | SNRNP70 | 0.40 | 0.27 | 0.24 | 1.70 | 1.70 |
| 92 | 22994 | CEP131 | 0.41 | 0.25 | 0.23 | 1.68 | 1.98 |
| 56 | 6626 | SNRPA | 0.43 | 0.26 | 0.23 | 1.52 | 1.94 |
| 91 | 201161 | CENPV | 0.41 | 0.23 | 0.41 | 1.36 | 1.41 |
| 50 | 8204 | NRIP1 | 0.34 | 0.35 | 0.23 | 1.01 | 1.42 |
| 85 | 891 | CCNB1 | 0.30 | 0.37 | 0.20 | 0.99 | 1.42 |
| 53 | 22913 | RALY | 0.39 | 0.23 | 0.16 | 0.92 | 1.57 |
| 34 | 2243 | FGA | 0.17 | 0.36 | 0.22 | 0.74 | 1.13 |
| 87 | 811 | CALR | 0.14 | 0.36 | 0.20 | 0.50 | 1.16 |
| 89 | 2771 | GNAI2 | −0.39 | −0.31 | −0.02 | 0.33 | 1.09 |
| 52 | 26525 | IL36RN | −0.36 | −0.30 | −0.06 | 0.30 | 1.12 |
| 54 | 57402 | S100A14 | −0.38 | −0.15 | −0.14 | −0.12 | 0.94 |
| 20 | 4314 | MMP3 | −0.35 | −0.35 | −0.22 | −0.49 | 0.87 |
| 86 | 6464 | SHC1 | −0.20 | −0.37 | −0.24 | −0.77 | 0.86 |
| 13 | 1457 | CSNK2A1 | 0.21 | 0.35 | −0.07 | −1.05 | 0.63 |
| 82 | 1676 | DFFA | −0.17 | −0.12 | −0.40 | −1.80 | 0.60 |
| 78 | 3915 | LAMC1 | −0.03 | −0.11 | −0.29 | −1.82 | 0.41 |
| 6 | 6279 | S100A8 | −0.26 | 0.07 | −0.14 | −1.87 | 0.64 |
| 77 | 3065 | HDAC1 | −0.11 | −0.14 | −0.22 | −1.90 | 0.56 |
| 79 | 4436 | MSH2 | −0.12 | −0.11 | −0.11 | −2.05 | 0.43 |
| 75 | 1048 | CEACAM5 | −0.02 | 0.00 | −0.30 | −2.13 | 0.56 |
| 83 | 1719 | DHFR | −0.31 | −0.28 | −0.33 | −2.25 | 0.59 |
| 74 | 408 | ARRB1 | −0.28 | −0.16 | −0.33 | −2.92 | 0.31 |

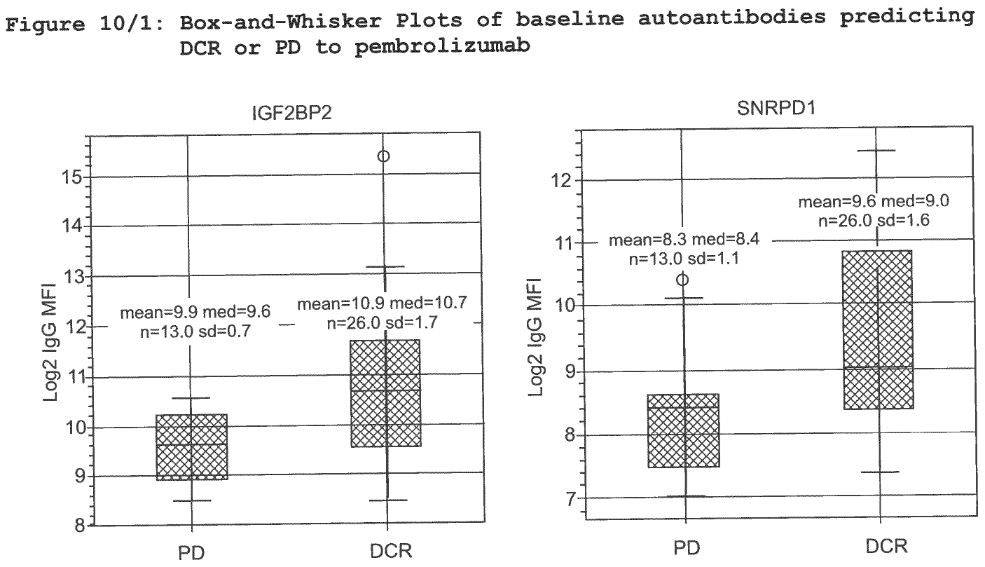

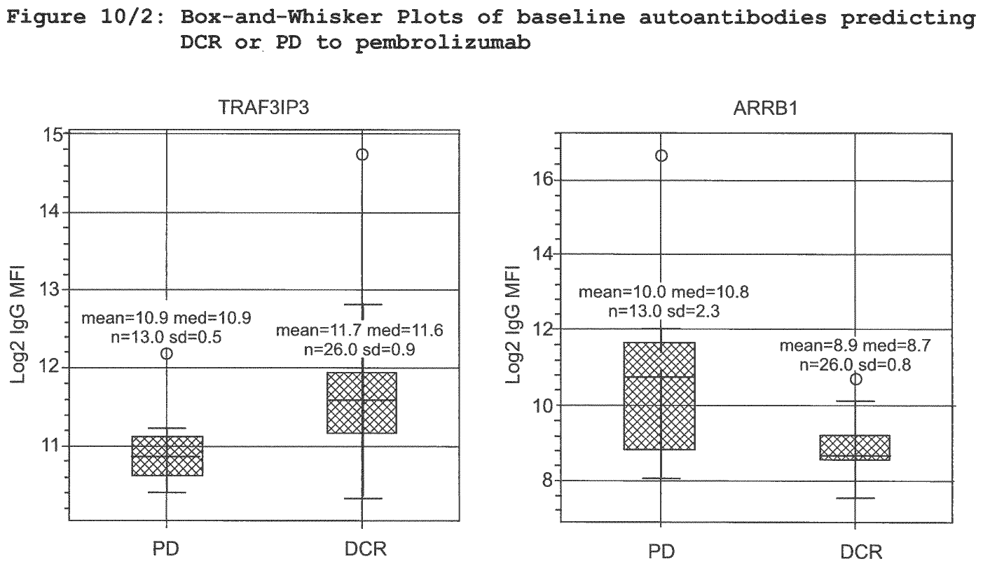

FIG. 10 shows Box-and-Whisker plots of four baseline autoantibodies targeting IGF2BP2, SNRPD1, TRAF3IP3, and ARRB1 predicting DCR or PD to pembrolizumab.

Higher baseline anti-IGFBP2 levels were found in patients who achieve DCR compared to patients with PD following pembrolizumab treatment. Furthermore, elevated levels of baseline anti-IGFBP2 autoantibodies predict clinical response as defined as DCR in patients treated with different checkpoint inhibitors (Table 3).

TRAF3IP3 is also known as “TRAF3-interacting JNK-activating modulator”, “TRAF3-interacting protein 3”, or T3JAM. TRAF3IP3 is specifically expressed in immune organs and tissues and plays a role in T and/or B cell development (Peng et al., 2015). Anti-TRAF3IP3 antibody levels were positively associated with survival (r=0.41) and PFS (r=0.49) and were increased in patients with DCR compared to patients with PD following pembrolizumab treatment.

SNRPD1 is also known as “small nuclear ribonucleoprotein Sm D1”, snRNP core protein D1, and is core component small nuclear ribonucleoprotein (snRNP) complexes. SNRPD1 or Sm-D1 is a known autoantigen and autoantibodies against this protein are specifically associated with the autoimmune disease systemic lupus erythematosus (SLE). Anti-SNRPD1 antibody levels were positively associated with survival (r=0.37) and were increased in patients with DCR compared to patients with PD following pembrolizumab treatment.

ARRB1 is also known as “beta-arrestin-1”, or ARR1. ARRB1 is is critical for CD4+ T cell survival and is a factor in susceptibility to autoimmunity (Shi et al., 2007). Higher anti-ARRB1 antibodies are found in baseline samples of melanoma patients with clinical non-response (PD) compared to patients with DCR to pembrolizumab therapy.

### Example 14

### Identification of Autoantibodies Associated with irAEs in Patients Treated with Pembrolizumab

Table 8 lists 35 baseline autoantibodies that are associated with the development of irAEs in patients treated with pembrolizumab.

To evaluate the difference in autoantibody levels between patients experiencing an irAE and those who do not, the statistical test SAM was applied. A positive SAM score-d and fold-change greater than 1 indicates that the autoantibody is elevated in the melanoma group who achieved DCR compared to patients who have had PD. A negative SAM score-d and fold-change less than 1 indicates that the autoantibody levels are lower in the melanoma group achieving DCR compared those who have had PD.

Twenty-seven autoantibodies show higher reactivity in baseline samples of patients who develop an irAE compared to patients without irAE and predict the development of irAE: FADD, OGT, HSPB1, CAP2, FN1, CTSW, ATP13A2, SIGIRR, TEX264, HSPA1B, SPTB, PDCD6IP, MITF, RAPGEF3, KRT7, ERBB3, PECAM1, PPL, TONSL, ELMO2, LAMB2, BTRC, SUFU, LGALS3BP, KLKB1, EGFR, and TOLLIP.

Eight baseline autoantibodies showed higher reactivity in the group of melanoma patients who do not develop irAE compared to patients without irAE: CXXC1, SPA17, LARP1, EGLN2, RPRM, WHSC1L1, MIF, and S100A8.

TABLE 8
Baseline autoantibodies predicting irAEs in melanoma
patients treated with pembrolizumab

| | | | SAM | SAM |
|---|---|---|---|---|
| | | Gene | Score.d. iRAE | Fold.Change |
| ID | Gene ID | Symbol | at T0 | irAE at T0 |
| 71 | 8772 | FADD | 3.08 | 2.83 |
| 48 | 8473 | OGT | 2.94 | 2.53 |
| 44 | 3315 | HSPB1 | 2.67 | 3.05 |
| 12 | 10486 | CAP2 | 2.59 | 2.35 |
| 121 | 2335 | FN1 | 2.55 | 2.40 |
| 69 | 1521 | CTSW | 2.43 | 2.30 |
| 68 | 23400 | ATP13A2 | 2.39 | 2.99 |
| 126 | 59307 | SIGIRR | 2.37 | 3.26 |
| 2 | 51368 | TEX264 | 2.31 | 3.14 |
| 17 | 3304 | HSPA1B | 2.14 | 2.37 |
| 18 | 6710 | SPTB | 2.14 | 2.37 |
| 35 | 10015 | PDCD6IP | 2.13 | 2.14 |
| 124 | 4286 | MITF | 2.13 | 2.48 |
| 65 | 10411 | RAPGEF3 | 2.12 | 3.48 |
| 122 | 3855 | KRT7 | 2.11 | 2.81 |
| 27 | 2065 | ERBB3 | 2.05 | 1.74 |
| 49 | 5175 | PECAM1 | 2.03 | 1.86 |
| 125 | 5493 | PPL | 2.01 | 2.12 |
| 130 | 4796 | TONSL | 1.98 | 2.44 |
| 29 | 63916 | ELMO2 | 1.89 | 1.98 |
| 123 | 3913 | LAMB2 | 1.89 | 2.25 |
| 119 | 8945 | BTRC | 1.87 | 2.05 |
| 128 | 51684 | SUFU | 1.87 | 1.81 |
| 47 | 3959 | LGALS3BP | 1.84 | 1.66 |
| 45 | 3818 | KLKB1 | 1.83 | 1.48 |
| 120 | 1956 | EGFR | 1.81 | 2.07 |
| 129 | 54472 | TOLLIP | 1.81 | 1.79 |
| 70 | 30827 | CXXC1 | −1.83 | 0.50 |
| 127 | 53340 | SPA17 | −1.85 | 0.43 |
| 46 | 23367 | LARP1 | −1.85 | 0.55 |
| 64 | 112398 | EGLN2 | −1.86 | 0.69 |
| 66 | 56475 | RPRM | −1.90 | 0.52 |
| 67 | 54904 | WHSC1L1 | −1.94 | 0.53 |
| 10 | 4282 | MIF | −2.11 | 0.54 |
| 6 | 6279 | S100A8 | −2.19 | 0.61 |

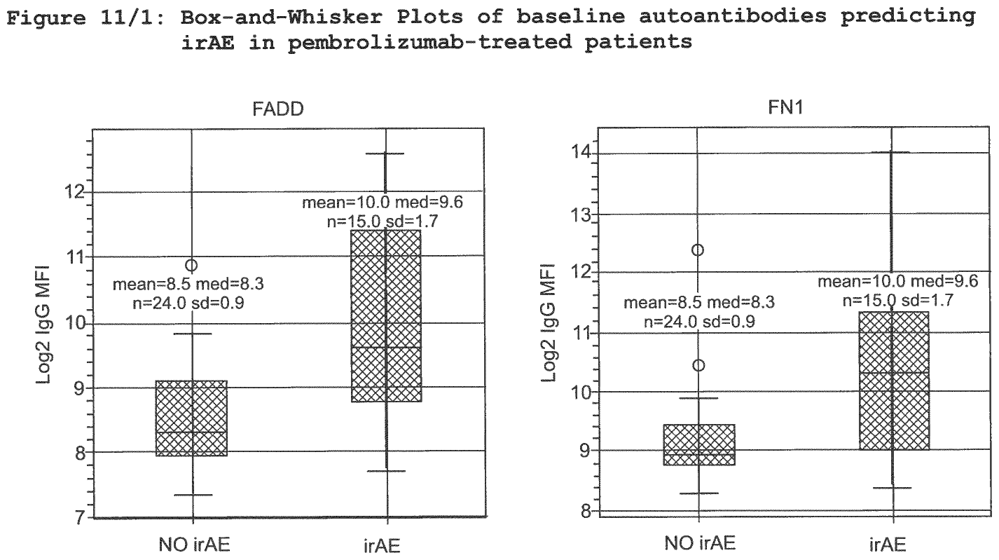

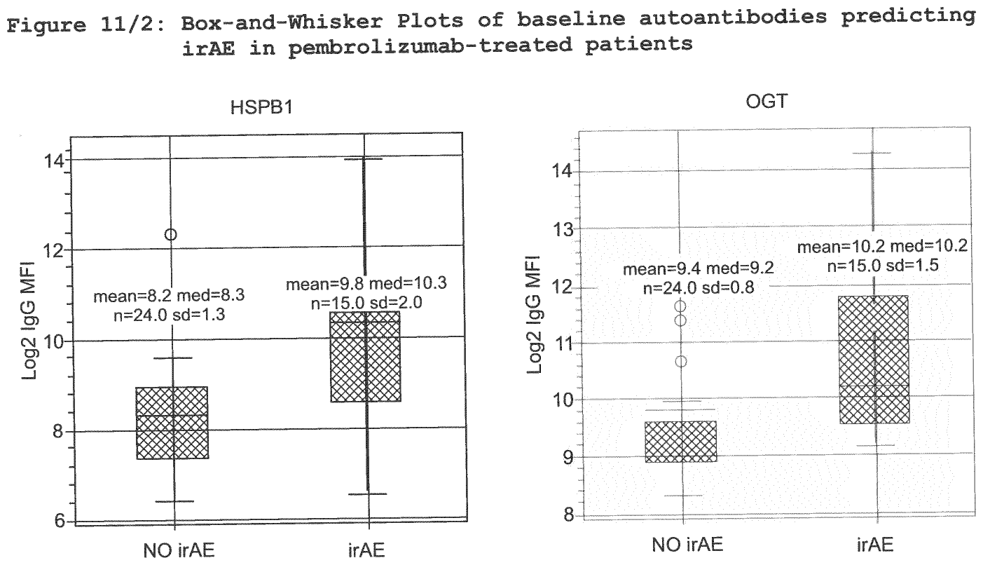

FIG. 11 shows Box-and-Whisker plots of four baseline autoantibody targets, FADD, FN1, HSPB1, and OGT, predicting irAE in pembrolizumab-treated patients.

Elevated autoantibodies directed against the pro-inflammatory cytokines S100A8 and MIF were found in melanoma patients who do not develop irAEs following pembrolizumab treatment.

MIF is also known as “Macrophage migration inhibitory factor (EC:5.3.2.1)”, “Glycosylation-inhibiting factor”, L-dopachrome tautomerase (EC:5.3.3.12), “Phenylpyruvate tautomerase”, GLIF, or MIF. MIF is a broad-spectrum proinflammatory cytokine, which plays a role in inflammatory and autoimmune diseases, but also has tumor-promoting effects (Kindt et al., 2016). Higher baseline anti-MIF1 antibody levels were found in patients who do not develop an irAE compared to those with an irAE following pembrolizumab treatment.

S100A8 is also known as “Protein S100-A8”, “Calgranulin-A”, “Calprotectin L1 L subunit”, “Migration inhibitory factor-related protein 8”, CFAG, or MRP8. S100A8 is a calcium- and zinc-binding protein, which plays a prominent role in the regulation of inflammatory processes and immune response. In many cancer types including melanoma, overexpression of 100A8 contributes to the growth, metastasis, angiogenesis and immune evasion of tumors (Bresnick et al., 2015). Higher baseline anti-S100A8 antibody levels were found in patients who do not develop an irAE compared to those with an irAE following pembrolizumab treatment.

Elevated levels of anti-S100A8 antibodies were also found in melanoma patients with progressive disease compared to patients with DCR following pembrolizumab (Table 7).

FADD is an also known as “FAS-associated death domain protein”, “Growth-inhibiting gene 3 protein”, “Mediator of receptor induced toxicity”, MORT1, or GIG3. FADD is an adaptor protein that bridges members of the tumor necrosis factor receptor superfamily, such as the Fas-receptor, to procaspases 8 and 10 to form the death-inducing signaling complex (DISC) during apoptosis. FADD has an important role in apoptosis, cell cycle regulation and cell survival, so that it can exert both tumor-suppressive and tumor-promoting roles. FADD is also is involved in inflammatory processes in autoimmune diseases (Cuda et al., 2016). Higher anti-FADD antibodies were found in patients who develop an irAE compared to those without irAE following treatment with pembrolizumab.

FN1 is also known as “Fibronectin”, “Cold-insoluble globulin”, or CIG. Fibronectin is a component of the extracellular matrix that plays a role in wound healing. In cancer, fibronectin promotes tumor growth/survival and resistance to therapy. Higher anti-FN1 antibodies were found in patients who develop an irAE compared to those without irAE following treatment with pembrolizumab.

HSBP1 is also known as “Heat shock protein beta-1”, “28 kDa heat shock protein”, “Estrogen-regulated 24 kDa protein”, “Heat shock 27 kDa protein”, HSP27, or HSP28. HSBP1 is a multifunctional protein, which acts as a protein chaperone and an antioxidant. In cancer, HSP27 plays a role in the inhibition of apoptosis. Higher anti-HSBP1 antibodies were found in patients who develop an irAE compared to those without irAE following treatment with pembrolizumab.

OGT is also known as “UDP-N-acetylglucosamine—peptide N-acetylglucosaminyltransferase 110 kDa subunit (EC:2.4.1.2554)”, or “O-GlcNAc transferase subunit p110”. OGT catalyzes the O-GlcNAcylation of a number of nuclear and cytoplasmic proteins thereby modulating cellular development and signaling pathways. Many cancer types display elevated O-GlcNAcylation and aberrant expression of OGT linking metabolism to invasion and metastasis (Ferrer et al., 2016).

Higher anti-OGT antibodies were found in patients who develop an irAE compared to those without irAE following treatment with pembrolizumab.

### Example 15

### Development of Biomarkers for Predicting the Risk to Develop an irAE

Multi-cohort metastatic melanoma samples for developing biomarker panels for irAE were obtained as follows.

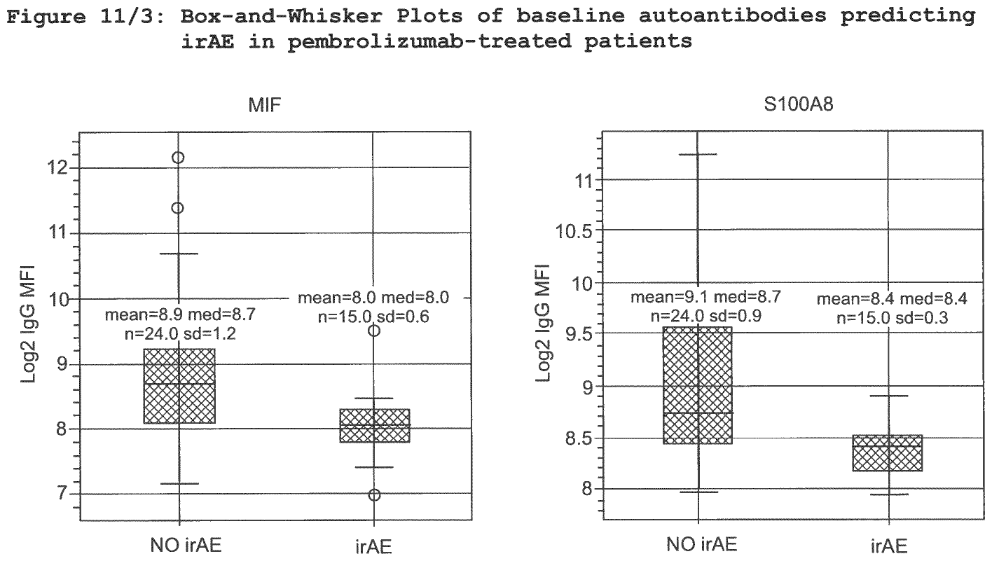

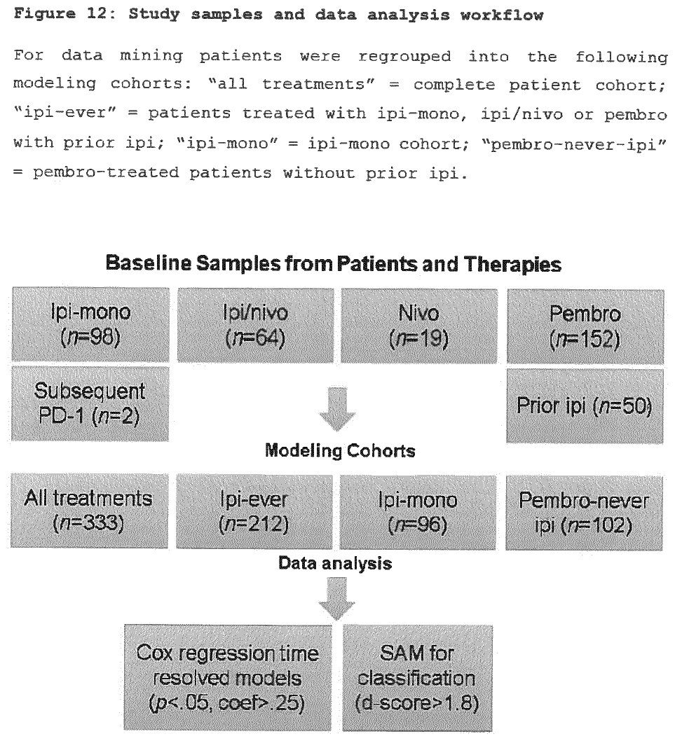

Serum samples from 333 metastatic melanoma patients were collected at 5 European cancer centers prior to treatment with the following therapeutic monoclonal antibodies ipilimumab (ipi, anti-CTLA-4), nivolumab (nivo, anti-PD-1), pembrolizumab (pembro, anti-PD-1), or ipilimumab with nivolumab combination therapy (FIG. 12). Serum samples were analyzed using a cancer immunotherapy antigen array (FIG. 1) comprising 832 antigens and were used to develop autoantibody biomarker panels for irAE and its subtype colitis.

All individuals provided written informed consent and the study was approved by the respective Ethics Committees. Patient data were provided including demographics (age, gender), treatment, date of therapy start, and best response (RECIST 1.1. criteria). Furthermore, irAEs were recorded including onset date and grade. As the risk for colitis might influence treatment choice in metastatic melanoma, namely the decision for anti-PD-1 monotherapy or ipi/nivo combination treatment we included colitis as an irAE of special interest.

The 333 included patients had a median age of 61 years, 38% were female. Overall, 103 patients (31%) developed irAEs including 44 patients with colitis (13%). Of the 98 patients who were treated with ipi monotherapy, 34 patients (35%) experienced an irAE of any grade and type, and 18 (18%) had colitis. Of 152 patients who were treated with pembrolizumab (pembro), 37 (24%) developed an irAE of any grade, 11 patients colitis (7%). 50 (32.9%) of pembro-treated patients had received ipi before, 14 patients (38%) of the irAE group and six (55%) of the colitis group.

Sixty-four patients were treated with ipi/nivo combination therapy of which 28 (44%) had any type of irAE and 15 had colitis (23%).

Statistical analysis for predicting the risk to develop an irAE was undertaken as follows.

To encode the different types of checkpoint inhibitors (anti-CTLA-4 and anti-PD-1) as a factor, we produced 5 modeling cohorts for data analysis (FIG. 12). We generated two CPI monotherapy groups (“ipi-mono” and “pembro-never-ipi”), which only include patients who received no other than the current CPI, and one combination therapy group “ipi/nivo”. Patients treated with ipi-mono, ipi-nivo or who have previously been treated with ipi were combined into the “ipi-ever” group. All 333 patients were also jointly investigated in the “all-treatments” analysis group.

To identify the most relevant biomarkers, we used a combination of linear and nonlinear data mining methods, which complement each other for feature selection. Significance Analysis of Microarrays (SAM) was used to compare patients according to the class label irAE or colitis. We used 1,000 permutations in a multiple testing approach for each autoantibody feature to ensure robust modeling. Feature ranking was achieved using the absolute value of the output d-score.

Candidate biomarkers were included in the set of final biomarker candidates using a threshold of the SAM score |d|>1.8. A positive SAM score-d and fold-change greater than 1 indicates that the autoantibody is elevated in patients who have had irAEs or colitis compared to those without irAEs or colitis. A negative SAM score-d and fold-change less than 1 indicates that the autoantibody level is lower in patients who have had irAEs or colitis compared to those without irAEs or colitis.

As a second approach for feature selection, Cox regression analysis was performed to investigate if pre-treatment autoantibody levels are related to the hazard ratio of an event using the R's survival package (https://cran.r-project.org/web/packages/survival/index.html). For Cox regression the treatment regime was included using the three treatment classes (PD-1, CTLA-4, PD-1+CTLA-4) as covariate factors. Within time-to-event, all relevant treatments with respect to the presence of PD-1 or CTLA-4 inhibition were considered in the covariate factor. The models were created in a one factor bottom up multiple testing approach (i.e. each biomarker was investigated one after another). For feature selection, we utilized the unadjusted p-value (p<0.05) of the Cox regression in combination with a minimum coefficient (coef>0.25). “Last contact” (and “death” for irAEs and colitis) were taken to censor the data to acknowledge data points from patients dropped out.

Kaplan Meier curves were calculated in combination with the Logrank test (using “survdiff” from R's survival package) for the same groups as for Cox regression, except for the all treatments group (http://www.sthda.com/english/rpkgs/survminer). Time-to-event was recorded starting at CPI therapy. The autoantibody data were dichotomized into autoantibody high versus low using the mean MFI value+1 SD of the healthy control sera as a marker-specific threshold.

As a complementary approach for feature ranking, Random Forests (RF) were calculated. We used a modification of the two-class classification method described by [10] using the “Tree-ensemble-learner” from KNIME. A number of 10,000 different models were generated. The tree depth was limited to 4 to investigate small panels with shallow trees, minimum split node size was 10 with minimum child node size of 5. The fraction of training data used for each model was 80% and attribute sampling was sampling a square root of total attributes combined with resampling for each tree node. Feature ranking was performed creating a score of the relative marker contribution for the first two levels of each tree.

Final feature ranking was performed by ranking markers according to their appearance in the respective tests. Final marker selection was performed to yield markers, which were above threshold in at least three tests.

Table 9 shows the top 47 autoantibodies predicting irAE or colitis.

The predictive autoantibody signature comprises the following antigen specificities:

SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT, SPTBN1, MUM1, RPLP2, KRT7, FN1, MAGEB4, CTSW, NCOA1, MIF, SPA17, FGFR1, KRT19, TPM2, ATG4D.

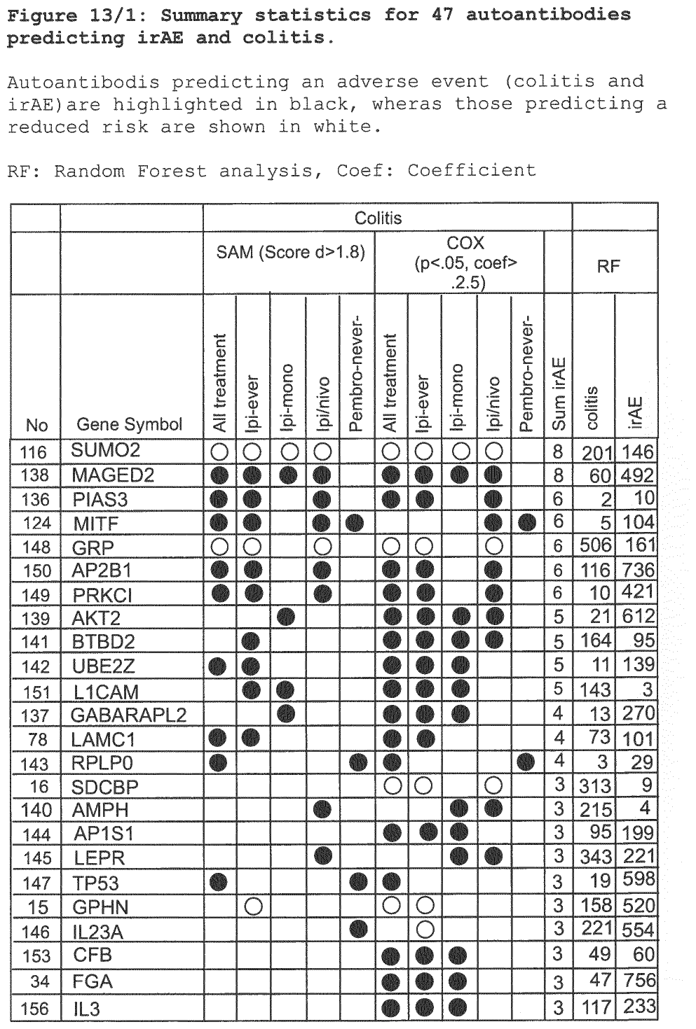

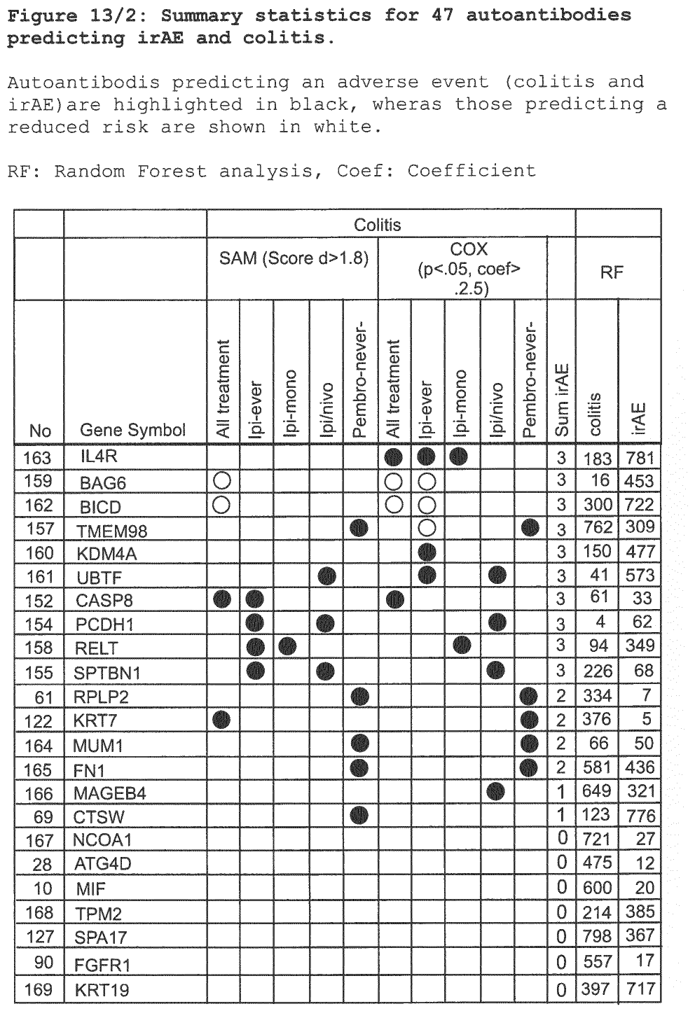

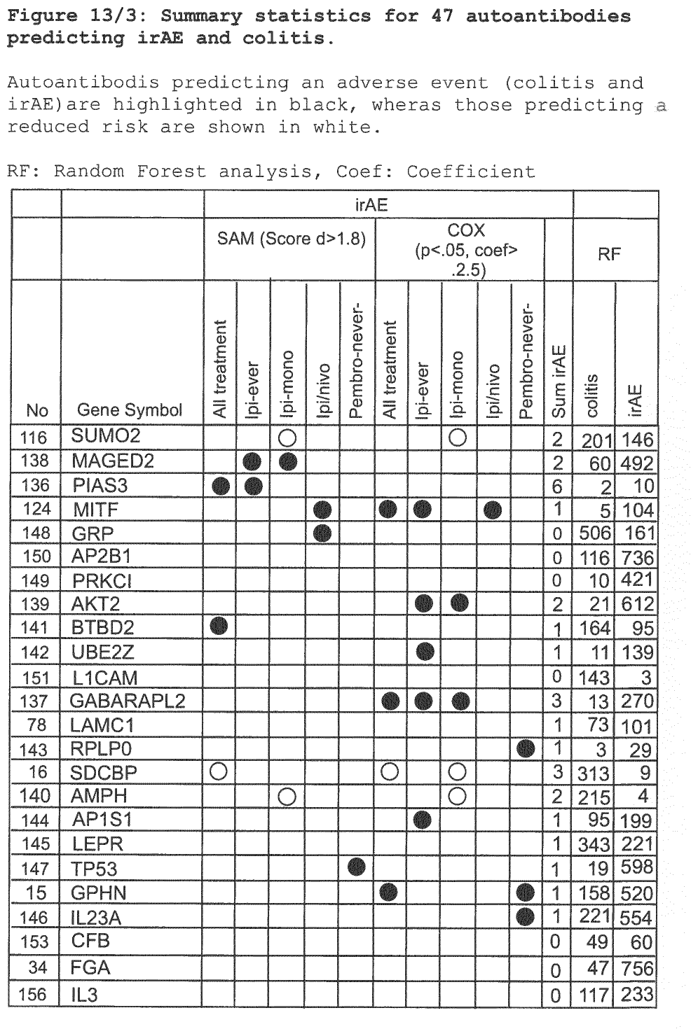

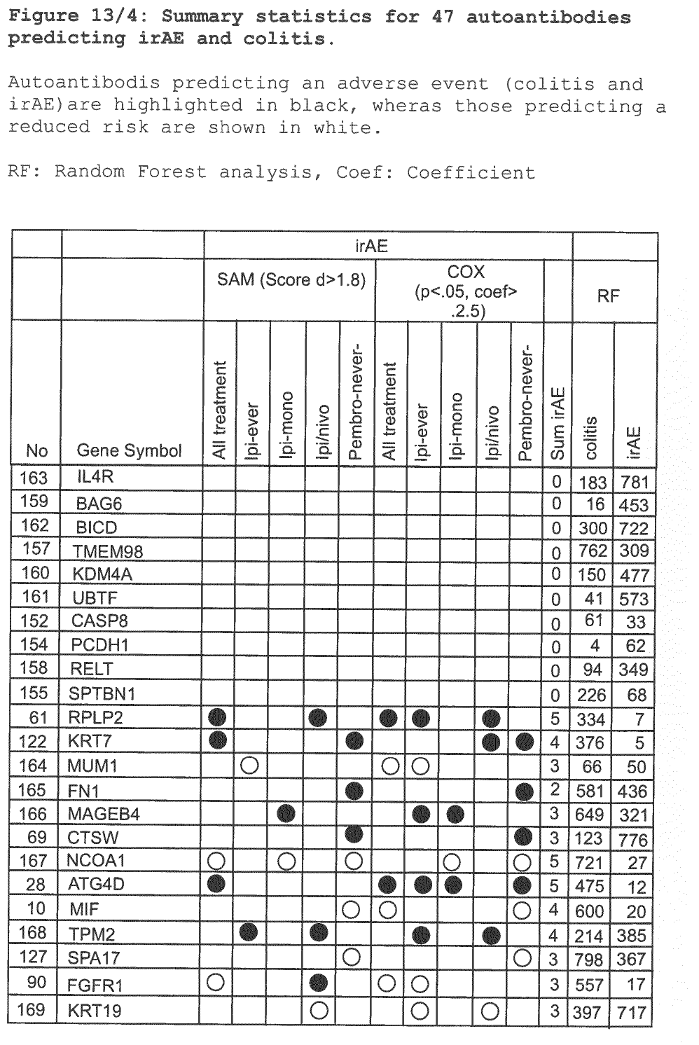

FIG. 13 summarizes the statistical test results and highlights autoantibodies that positively (black circles) or negatively (white circles) predict irAE or colitis.

TABLE 9
List of top 47 marker predicting irAE or colitis
The thresholds were: SAM analysis (Score d > 1.8) and
Cox regression analysis (p < 0.05, coefficient0 > .25)
in any of the modeling cohorts.

| | | Gene | | | | | |
|---|---|---|---|---|---|---|---|
| | | Symbol | | | | | |
| | | (and | | | | | |
| | | Exemplary | | | | | |
| | | Antigen | | Colitis | | irAE | |
| No | Gene ID | Sequence) | Gene name | SAM | Cox | SAM | Cox |
| 10 | 4282 | MIF | Macrophage migration | | | x | x |
| | | | inhibitory factor | | | | |
| 15 | 10243 | GPHN | Gephyrin | x | x | | x |
| 16 | 6386 | SDCBP | Syntenin-1 | | x | x | x |
| 28 | 84971 | ATG4D | Cysteine protease ATG4D | | | x | x |
| 34 | 2243 | FGA | Fibrinogen alpha chain | | x | | |
| 61 | 6181 | RPLP2 | 60S acidic | x | x | x | x |
| | | | ribosomal protein | | | | |
| | | | P2 | | | | |
| 69 | 1521 | CTSW | Cathepsin W | x | | x | x |
| 78 | 3915 | LAMC1 | Laminin subunit gamma-1 | x | x | x | |
| 90 | 2260 | FGFR1 | Fibroblast growth factor | | | x | x |
| | | | receptor 1 (CD331) | | | | |
| 116 | 6613 | SUMO2 | Small ubiquitin-related | x | x | x | x |
| | | | modifier 2 | | | | |
| 122 | 3855 | KRT7 | Cytokeratin-7 | x | x | x | x |
| 124 | 4286 | MITF | Microphthalmia-associated | x | x | x | |
| | | | transcription factor | | | | |
| 127 | 53340 | SPA17 | Sperm surface protein Sp17 | | | x | x |
| | | | (CT22) | | | | |
| 136 | 10401 | PIAS3 (SEQ | E3 SUMO-protein ligase | x | x | x | x |
| | | ID NO: 136) | PIAS3 | | | | |
| 137 | 11345 | GABARAPL | (GABA(A) receptor- | x | x | | x |
| | | 2(SEQ ID | associated protein-like 2 | | | | |
| | | NO: 137) | | | | | |
| 138 | 10916 | MAGED2 | Melanoma-associated antigen | x | x | x | |
| | | (SEQ ID NO: | D2 | | | | |
| | | 138) | | | | | |
| 139 | 208 | AKT2(SEQ | RAC-beta serine/threonine- | x | x | | x |
| | | ID NO: 139) | protein kinase | | | | |
| 140 | 273 | AMPH(SEQ | Amphiphysin | x | x | x | x |
| | | ID NO: 140) | | | | | |
| 141 | 55643 | BTBD2(SEQ | BTB/POZ domain-containing | x | x | x | |
| | | ID NO: 141) | protein 2 | | | | |
| 142 | 65264 | UBE2Z(SEQ | Ubiquitin-conjugating enzyme | x | x | | x |
| | | ID NO: 142) | E2Z | | | | |
| 143 | 6175 | RPLP0(SEQ | 60S acidic ribosomal protein | x | x | | x |
| | | ID NO: 143) | P0 | | | | |
| 144 | 1174 | AP1S1(SEQ | AP-1 complex subunit sigma- | | x | | x |
| | | ID NO: 144) | 1A | | | | |
| 145 | 3953 | LEPR(SEQ | Leptin receptor (CD295) | x | x | x | |
| | | ID NO: 145) | | | | | |
| 146 | 51561 | IL23A(SEQ | Interleukin-23 subunit alpha | x | x | | x |
| | | ID NO: 146) | (IL-23p19) | | | | |
| 147 | 7157 | TP53(SEQ | Cellular tumor antigen p53 | x | x | x | |
| | | ID NO: 147) | | | | | |
| 148 | 2922 | GRP(SEQ | Gastrin-releasing peptide | x | x | | |
| | | ID NO: 148) | | | | | |
| 149 | 5584 | PRKCI(SEQ | Protein kinase C iota type | x | x | | |
| | | ID NO: 149) | | | | | |
| 150 | 163 | AP2B1 (SEQ | AP-2 complex subunit beta | x | x | | |
| | | ID NO: 150) | | | | | |
| 151 | 3897 | L1CAM(SEQ | Neural cell adhesion molecule | x | x | | |
| | | ID NO: 151) | L1 (CD171) | | | | |
| 152 | 841 | CASP8(SEQ | Caspase-8 | x | x | | |
| | | ID NO: 152) | | | | | |
| 153 | 629 | CFB(SEQ ID | Complement factor B | | x | | |
| | | NO: 153) | | | | | |
| 154 | 5097 | PCDH1(SEQ | Protocadherin-1 | x | x | | |
| | | ID NO: 154) | | | | | |
| 155 | 6711 | SPTBN1 | Spectrin beta chain | x | x | | |
| | | (SEQ ID NO: | | | | | |
| | | 155) | | | | | |
| 156 | 3562 | IL3(SEQ ID | lnterleukin-3 | | x | | |
| | | NO: 156) | | | | | |
| 157 | 26022 | TMEM98 | Transmembrane protein 98 | x | x | | |
| | | (SEQ ID NO: | (Protein TADA1) | | | | |
| | | 157) | | | | | |
| 158 | 84957 | RELT(SEQ | Tumor necrosis factor | x | x | | |
| | | ID NO: 158) | receptor superfamily member | | | | |
| | | | 19L | | | | |
| 159 | 7917 | BAG6(SEQ | Large proline-rich protein | x | x | | |
| | | ID NO: 159) | BAG6 | | | | |
| 160 | 9682 | KDM4A | Lysine-specific demethylase | x | x | | |
| | | (SEQ ID NO: | 4A | | | | |
| | | 160) | | | | | |
| 161 | 7343 | UBTF(SEQ | Nucleolar transcription factor | x | x | | |
| | | ID NO: 161) | 1 (Autoantigen NOR-90) | | | | |
| 162 | 23299 | BICD2(SEQ | Protein bicaudal D homolog 2 | x | x | | |
| | | ID NO: 162) | | | | | |
| 163 | 3566 | IL4R(SEQ ID | lnterleukin-4 receptor subunit | | x | | |
| | | NO: 163) | alpha (CD124) | | | | |
| 164 | 84939 | MUM1 (SEQ | Mutated melanoma- | x | x | x | x |
| | | ID NO: 164) | associated antigen 1 | | | | |
| 165 | 2335 | FN1(SEQ ID | Fibronectin | x | x | x | x |
| | | NO: 165) | | | | | |
| 166 | 4115 | MAGEB4 | Melanoma-associated antigen | | x | x | x |
| | | (SEQ ID NO: | B4 | | | | |
| | | 166) | | | | | |
| 167 | 8648 | NCOA1 | Nuclear receptor coactivator 1 | | | x | x |
| | | (SEQ ID NO: | | | | | |
| | | 167) | | | | | |
| 168 | 7169 | TPM2(SEQ | Tropomyosin beta chain | | | x | x |
| | | ID NO: 168) | | | | | |
| 169 | 3880 | KRT19(SEQ | Cytokeratin-19 | | | x | x |
| | | ID NO: 169) | | | | | |

Association rule mining was performed using the software Natto Ef Prime Inc. (Japan) and network graphs with corresponding associations were created. Autoantibody intensity data were categorized into 3 categories (low, medium and high intensity). We computed a description score as an index, which represents the proportion of uncertainty in Y that X can explain for each edge in the network as mutual information. We selected irAE and colitis as targets to highlight the relevant attributes, which have the highest description scores (mutual information) in the model.

### Example 16

### Exploration of an Autoantibody Signature for Prediction of Colitis

SAM analysis and Cox regression analysis was performed to identify autoantibody reactivities associated with the development of colitis following checkpoint inhibitor treatment. The following treatment groups were investigated: All 333 patients were combined into the “all-treatments” group, “ipi-mono”, “ipi-nivo” combination therapy, “ipi-ever” and “pembro-never-ipi” group. This analysis yielded 34 autoantibodies for predicting colitis, which were found in at least three group comparisons as shown in FIG. 13.

The results of the Cox regression analysis and the associated hazard risk (HR) for developing an irAE in patients with high autoantibody levels is shown in Table 10 for the autoantibody signature predicting irAE and colitis.

The 34 autoantibodies comprise the following antigen specificities: SUMO2, MAGED2, PIAS3, MITF, GRP, AP2B1, PRKCI, BTBD2, AKT2, UBE2Z, L1CAM, LAMC1, GABARAPL2, RPLP0, SDCBP, AP1S1, CFB, FGA, IL3, IL4R, AMPH, LEPR, TP53, GPHN, IL23A, BAG6, BICD2, TMEM98, KDM4A, UBTF, CASP8, PCDH1, RELT and SPTBN1.

Six of the 34 autoantibody reactivities were associated with a reduced risk to develop colitis following checkpoint inhibitor treatment. Patients without colitis had higher autoantibody levels compared to those with colitis: SUMO2, GRP, SDCBP, GPHN, BAG6, and BICD2.

26 of the 34 autoantibody reactivities were associated with a higher risk to develop colitis.

Following checkpoint inhibitor treatment, patients who have had colitis had higher autoantibody levels compared to patients without colitis comprise the following antigen specificities: MAGED2, PIAS3, MITF, AP2B1, PRKCI, BTBD2, AKT2, UBE2Z, L1CAM, LAMC1, GABARAPL2, RPLP0, AP1S1, CFB, FGA, IL3, IL4R, AMPH, LEPR, TP53, KDM4A, UBTF, CASP8, PCDH1, RELT, and SPTBN1.

There were two autoantibodies anti-TMEM98 and anti-IL23A that were associated with a reduced risk to develop colitis in patients of the “ipi-ever” group, but were elevated in baseline samples of patients who have had colitis when treated with pembro.

30 of the 34 antigens were identified in the ipi-ever group, demonstrating a strong association of anti-CTLA-4 therapy with the development of colitis (FIG. 13, Table 10).

However, there were also differences seen in autoantibody patterns between ipi-mono and ipi/nivo combination therapy. For example, autoantibodies against UBE2Z, L1CAM, GABARAPL2, CFB, IL3, RELT, FGA, and IL4R predict colitis in the ipi-mono group, whereas autoantibodies targeting PIAS3, SUMO2, MITF, GRP, PRKCI, AP2B1, SDCBP, PDCH1, SPTBN1, and UBTF were predictive in the ipi/nivo cohort. The autoantibodies with the highest score for predicting colitis were MAGED2, PIAS3, MITF, PRKC1, and AP2B1 (FIG. 13). Two high scoring markers predicted a reduced risk to develop colitis, which were autoantibodies targeting SUMO2, and GRP.

The marker with the highest score for predicting colitis was anti-MAGED2 with significant associations found for the “all treatment” (HR 1.35, p=0.002), “ipi-ever” (HR 1.36, p=0.0012), (ipi-mono” (HR 1.48, p=0.024), and “ipi/nivo group” (HR 1.31, p=0.036). The marker with the smallest p-value in the ipi-ever group was anti-PIAS3 with significant associations for the all treatment (HR 1.42, p=0.00005), ipi-ever (HR 1.46, p=0.000009), and ipi/nivo group (HR 1.52, p=0.0004).

Higher levels of anti-SUMO2 autoantibodies predicted a lower risk to develop colitis in the “all treatment” (HR 0.53, p=0.0022), “ipi-ever” (HR 0.51, p=0.0026), “ipi-mono” (HR 0.32, p=0.0012), and “ipi/nivo” group (HR 0.5, p=0.049).

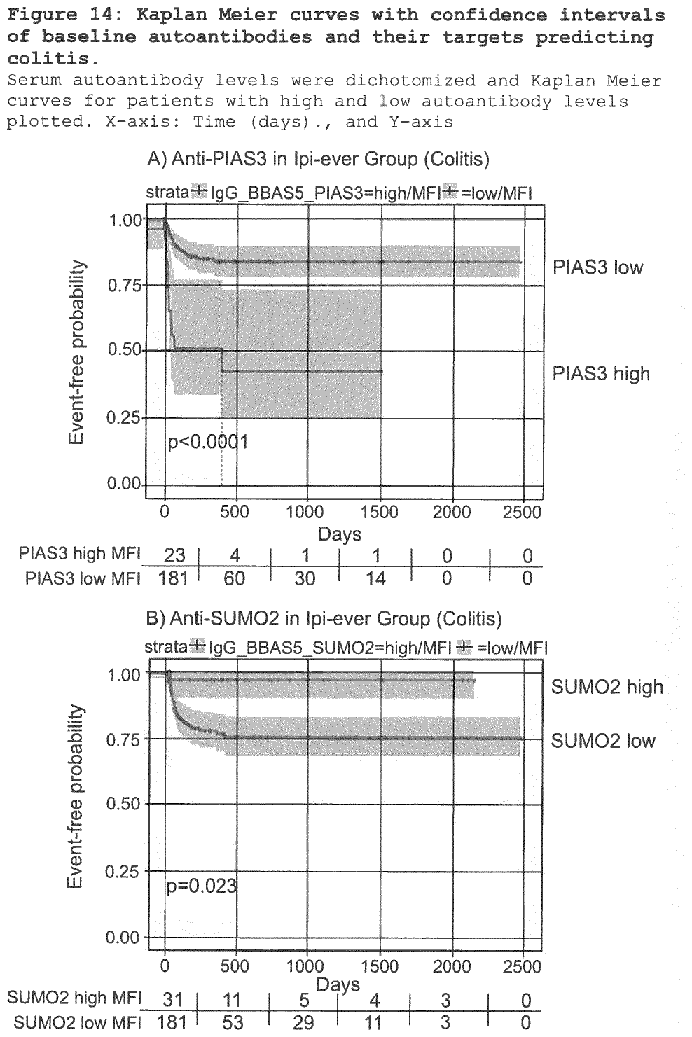

FIG. 14 shows examples of Kaplan-Meier curves for anti-PIAS3 and anti-SUM02 in the “ipi-ever” group and the risk to develop colitis. Patients with higher baseline anti-PIAS3 autoantibody levels had an increased risk to develop irAEs compared to those with lower autoantibody levels.

Patients with higher baseline anti-SUMO2 autoantibody levels had a reduced risk to develop colitis compared to those with lower autoantibody levels.

TABLE 10
Results of Cox regression analysis of autoantibodies predicting colitis
p-values < 0.05 are highlighted in bold; HR = hazard ratio

| | All | | | | | | | | Pembro | |
|---|---|---|---|---|---|---|---|---|---|---|
| Gene | Treatments | | Ipi Ever | | Ipi Mono | | Ipi/Nivo | | Never Ipi | |
| Symbol of | P- | | P- | | P- | | P- | | P- | |
| Antigen | value | HR | value | HR | value | HR | value | HR | value | HR |
| AKT2 | 0.0006 | 1.75 | 0.0008 | 1.80 | 0.0006 | 2.25 | 0.0307 | 1.96 | 0.1640 | 1.95 |
| AMPH | 0.3562 | 1.09 | 0.3764 | 1.09 | 0.0449 | 0.75 | 0.0020 | 1.70 | 0.4250 | 1.26 |
| AP1S1 | 0.0344 | 1.49 | 0.0124 | 1.58 | 0.0451 | 1.62 | 0.2169 | 1.45 | 0.2509 | 0.31 |
| AP2B1 | 0.0040 | 1.33 | 0.0092 | 1.30 | 0.5221 | 1.19 | 0.0165 | 1.31 | 0.2165 | 1.74 |
| ATG4D | 0.9413 | 1.02 | 0.6092 | 1.14 | 0.2384 | 1.44 | 0.7893 | 1.15 | 0.3340 | 0.36 |
| BAG6 | 0.0386 | 0.71 | 0.0430 | 0.69 | 0.1749 | 0.69 | 0.2718 | 0.76 | 0.5946 | 0.79 |
| BICD2 | 0.0128 | 0.58 | 0.0354 | 0.63 | 0.2746 | 0.67 | 0.0611 | 0.49 | 0.1658 | 0.30 |
| BTBD2 | 0.0052 | 1.37 | 0.0015 | 1.41 | 0.0205 | 1.47 | 0.0369 | 1.34 | 0.3489 | 0.55 |
| CASP8 | 0.0069 | 1.30 | 0.0175 | 1.28 | 0.2020 | 1.21 | 0.4043 | 1.23 | 0.2672 | 1.38 |
| CFB | 0.0296 | 2.00 | 0.0147 | 2.16 | 0.0029 | 2.73 | 0.6696 | 1.33 | 0.5528 | 0.41 |
| CTSW | 0.4987 | 1.11 | 0.6748 | 0.92 | 0.3918 | 0.75 | 0.3826 | 0.74 | 0.2066 | 1.38 |
| FGA | 0.0430 | 1.56 | 0.0135 | 1.72 | 0.0137 | 2.45 | 0.3334 | 1.46 | 0.1526 | 0.18 |
| FGFR1 | 0.1905 | 0.72 | 0.1950 | 0.71 | 0.4259 | 0.78 | 0.3725 | 0.61 | 0.8267 | 0.84 |
| FN1 | 0.2507 | 1.20 | 0.9314 | 1.02 | 0.8925 | 0.95 | 0.5645 | 0.78 | 0.0356 | 2.05 |
| GABARAPL2 | 0.0140 | 1.62 | 0.0036 | 1.84 | 0.0035 | 2.09 | 0.5476 | 0.70 | 0.5252 | 0.58 |
| GPHN | 0.0203 | 0.57 | 0.0062 | 0.48 | 0.0539 | 0.49 | 0.0583 | 0.34 | 0.4375 | 1.45 |
| GRP | 0.0025 | 0.71 | 0.0048 | 0.71 | 0.0696 | 0.69 | 0.0473 | 0.66 | 0.2100 | 0.50 |
| IL23A | 0.6077 | 0.90 | 0.0333 | 0.55 | 0.0928 | 0.41 | 0.1936 | 0.60 | 0.0232 | 1.86 |
| IL3 | 0.0258 | 1.31 | 0.0361 | 1.31 | 0.0351 | 1.56 | 0.7551 | 0.91 | 0.0928 | 2.41 |
| IL4R | 0.0444 | 1.74 | 0.0398 | 1.77 | 0.0283 | 2.15 | 0.1662 | 2.15 | 0.7443 | 0.72 |
| KDM4A | 0.0085 | 1.39 | 0.0027 | 1.45 | 0.4092 | 1.35 | 0.3540 | 1.18 | 0.5818 | 0.68 |
| KRT19 | 0.5878 | 0.93 | 0.4218 | 0.88 | 0.7679 | 0.92 | 0.0944 | 0.58 | 0.4050 | 1.31 |
| KRT7 | 0.0166 | 1.25 | 0.0620 | 1.21 | 0.8772 | 1.03 | 0.1078 | 1.27 | 0.0360 | 1.74 |
| L1CAM | 0.0482 | 1.29 | 0.0171 | 1.38 | 0.0082 | 1.65 | 0.2069 | 1.36 | 0.4026 | 0.63 |
| LAMC1 | 0.0017 | 1.31 | 0.0015 | 1.33 | 0.1737 | 1.19 | 0.2561 | 1.18 | 0.8534 | 1.05 |
| LEPR | 0.1914 | 1.11 | 0.0730 | 1.17 | 0.0397 | 1.29 | 0.0357 | 1.31 | 0.6056 | 0.86 |
| MAGEB4 | 0.3248 | 1.11 | 0.1395 | 1.18 | 0.5536 | 1.11 | 0.0237 | 1.36 | 0.4077 | 0.68 |
| MAGED2 | 0.0020 | 1.35 | 0.0012 | 1.36 | 0.0241 | 1.38 | 0.0361 | 1.31 | 0.5827 | 0.65 |
| MIF | 0.0902 | 0.67 | 0.1591 | 0.71 | 0.4832 | 0.81 | 0.2980 | 0.57 | 0.3325 | 0.53 |
| MITF | 0.0018 | 1.28 | 0.0217 | 1.22 | 0.7467 | 0.95 | 0.0007 | 1.47 | 0.0484 | 1.54 |
| MUM1 | 0.3432 | 1.13 | 0.6524 | 0.93 | 0.4233 | 0.80 | 0.3924 | 1.19 | 0.0098 | 1.71 |
| NCOA1 | 0.0912 | 0.85 | 0.1552 | 0.87 | 0.1005 | 0.75 | 0.4389 | 1.11 | 0.1812 | 0.57 |
| PCDH1 | 0.0916 | 1.13 | 0.0204 | 1.20 | 0.2108 | 1.14 | 0.0147 | 1.35 | 0.2909 | 0.70 |
| PIAS3 | 0.0000 | 1.42 | 0.0000 | 1.46 | 0.2308 | 1.22 | 0.0004 | 1.52 | 0.8245 | 0.88 |
| PRKCI | 0.0002 | 1.39 | 0.0001 | 1.41 | 0.0601 | 1.35 | 0.0014 | 1.44 | 0.5171 | 0.64 |
| RELT | 0.0249 | 1.19 | 0.0116 | 1.23 | 0.0289 | 1.29 | 0.7263 | 0.92 | 0.9069 | 0.96 |
| RPLP0 | 0.0102 | 1.40 | 0.2227 | 1.22 | 0.0591 | 1.36 | 0.6500 | 0.79 | 0.0007 | 3.26 |
| RPLP2 | 0.1858 | 1.22 | 0.5715 | 1.10 | 0.3298 | 1.32 | 0.8022 | 1.06 | 0.0132 | 2.43 |
| SDCBP | 0.0068 | 0.44 | 0.0304 | 0.52 | 0.3783 | 0.73 | 0.0271 | 0.27 | 0.1167 | 0.24 |
| SPA17 | 0.8799 | 1.01 | 0.7909 | 1.02 | 0.2891 | 0.85 | 0.3274 | 1.13 | 0.9692 | 1.01 |
| SPTBN1 | 0.0460 | 1.21 | 0.0245 | 1.23 | 0.7310 | 1.05 | 0.0218 | 1.39 | 0.4612 | 0.79 |
| SUMO2 | 0.0022 | 0.53 | 0.0026 | 0.51 | 0.0093 | 0.32 | 0.0488 | 0.50 | 0.9235 | 0.96 |
| TMEM98 | 0.1323 | 0.85 | 0.0207 | 0.73 | 0.0516 | 0.64 | 0.5518 | 0.88 | 0.0107 | 1.77 |
| TP53 | 0.0118 | 1.29 | 0.4205 | 1.12 | 0.3735 | 1.15 | 0.5922 | 0.82 | 0.1227 | 1.29 |
| TPM2 | 0.9090 | 0.99 | 0.8150 | 1.03 | 0.9717 | 1.01 | 0.8859 | 1.03 | 0.2451 | 0.58 |
| UBE2Z | 0.0001 | 1.74 | 0.0000 | 1.79 | 0.0144 | 1.78 | 0.0976 | 1.41 | 0.2703 | 0.24 |
| UBTF | 0.0538 | 1.39 | 0.0119 | 1.52 | 0.8610 | 1.07 | 0.0214 | 1.53 | 0.0796 | 0.17 |

### Example 17

### Exploration of an Autoantibody Signature Predicting irAE

SAM analysis and Cox regression analysis was performed to identify autoantibody reactivities associated with the development of irAEs following checkpoint inhibitor treatment. The following treatment groups were investigated: All 333 patients were combined into the “all-treatments” group, “ipi-mono”, “ipi-nivo” combination therapy, “ipi-ever” and “pembro-never-ipi” group.

A feature ranking approach was applied to select the 15 most important biomarker candidates for irAE, which were found in at least three group comparisons as shown in FIG. 13. The results of the Cox regression analysis and the associated hazard risk (HR) for developing an irAEs for the different treatment groups is shown in Table 11.

The 15 most important autoantibody specificities for predicting an irAE comprise the following antigens: PIAS3, RPLP2, NCOA1, ATG4D, KRT7, MIF, TPM2, GABARAPL2, SDCBP, MUM1, MAGEB4, CTSW, SPA17, FGFR1, KRT19.

Seven of the 15 autoantibodies were associated with an increased risk of irAE and target the following antigens: PIAS3, RPLP2, ATG4D, KRT7, TPM2, GABARAPL2, and MAGEB4. Patients who have had IrAEs had higher autoantibody levels to these antigens compared to patients without irAEs. Six of the 15 autoantibodies were associated with a reduced risk of irAE and target the following antigens: NCOA1, MIF, SDCB4, MUM1, FGFR1, and KRT19. Patients who have had IrAEs had higher autoantibody levels to these antigens compared to patients without irAEs.

Therapy-related differences were also observed, for example anti-KRT7 and anti-FN1 were only predictive in anti-PD-1 treated patients, which comprise the “pembro-never-ipi” and “ipi/nivo” groups. Anti-MAGEB4 and anti-MAGED2 were preferentially predictive in anti-CTLA-4 therapies, which comprise the “ipi-mono” and “ipi-ever” treatment groups.

The top biomarker for irAE associated with anti-CTLA-4 therapy was anti-PIAS3 antibodies with significant associations found for the “all treatment group” (HR 1.29, p=0.0001), the “ipi-ever” group (HR 1.29, p=0.0002; HR 1.35) and the “ipi/nivo” group (HR 1.32, p=0.0035).

The top biomarker for irAE associated with anti-PD1 therapy was anti-KRT7 with significant associations found for the ipi/nivo group (HR 1.31, p=0.04) and pembro-never-ipi group (HR 1.55, p=0.0008).

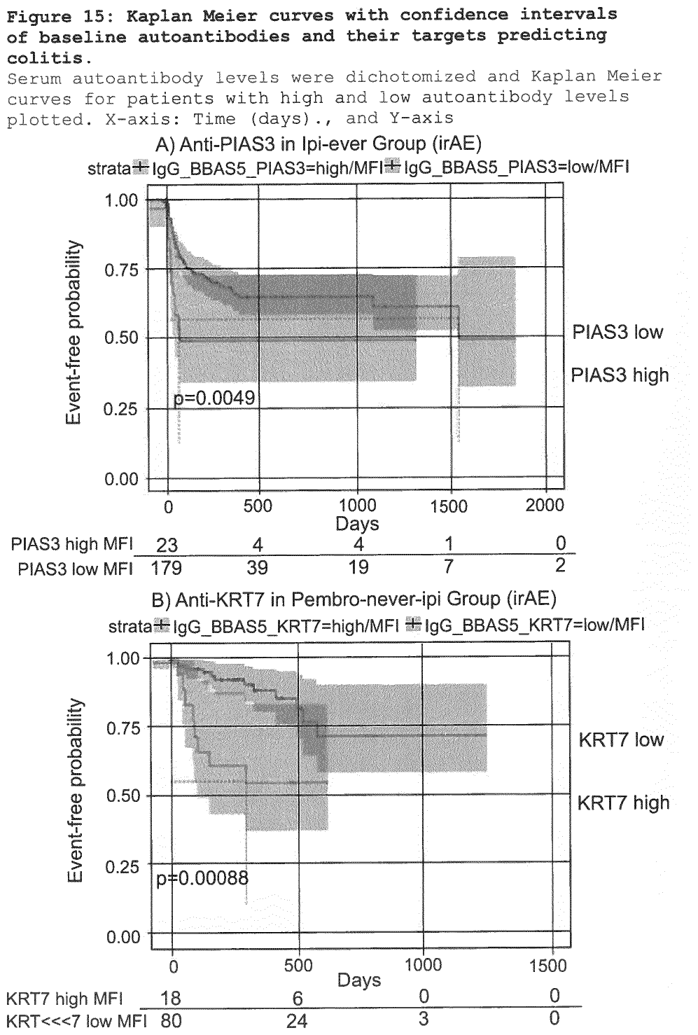

FIG. 15 shows examples of Kaplan-Meier curves for irAE and anti-PIAS3 and anti-KRT7 antibodies.

Patients with higher baseline anti-PIAS3 and anti-KRT7 autoantibody levels had an increased risk to develop irAEs compared to patients with lower autoantibody levels.

Therapy-related differences were found for autoantibodies predicting a reduced risk of irAE.

Whereas anti-MUM1 (HR 0.69, p=0.0074) and anti-FGFR1 (HR 0.69, p=0.037) antibodies were associated with anti-CTLA-4 therapy (“ipi-ever” group), anti-MIF1 antibodies predicted a reduced risk of irAE for the “pembro-never-ipi” group (HR 0.49, p=0.032).

TABLE 11
Results of Cox regression analysis of autoantibodies predicting irAE
p-values < 0.05 are highlighted in bold; HR = hazard ratio

| | | | | | | | | | Pembro | |
|---|---|---|---|---|---|---|---|---|---|---|
| Gene | All | | | | Ipi Mono | | Ipi/Nivo | | Never Ipi | |
| Symbol of | Treatments | | Ipi Ever | | P- | | P- | | P- | |
| Antigen | P-value | HR | P-value | HR | value | HR | value | HR | value | HR |
| AKT2 | 0.3314 | 1.16 | 0.0328 | 1.44 | 0.0016 | 2.00 | 0.1799 | 1.54 | 0.2841 | 0.64 |
| AMPH | 0.1705 | 0.92 | 0.6275 | 0.97 | 0.0120 | 0.78 | 0.0835 | 1.24 | 0.1184 | 0.82 |
| AP1S1 | 0.1047 | 1.26 | 0.0341 | 1.39 | 0.0674 | 1.50 | 0.5742 | 1.16 | 0.4057 | 0.72 |
| AP2B1 | 0.0991 | 1.17 | 0.0577 | 1.20 | 0.9027 | 1.03 | 0.0578 | 1.22 | 0.8031 | 0.92 |
| ATG4D | 0.0007 | 1.38 | 0.0104 | 1.47 | 0.0002 | 2.85 | 0.7261 | 1.14 | 0.0102 | 1.41 |
| BAG6 | 0.4876 | 0.95 | 0.2034 | 0.88 | 0.1664 | 0.78 | 0.6080 | 0.93 | 0.4482 | 1.12 |
| BICD2 | 0.2381 | 1.12 | 0.3004 | 1.12 | 0.9776 | 0.99 | 0.2951 | 1.17 | 0.4416 | 1.14 |
| BTBD2 | 0.0059 | 1.27 | 0.0112 | 1.27 | 0.1549 | 1.26 | 0.0615 | 1.25 | 0.1832 | 1.37 |
| CASP8 | 0.0216 | 1.21 | 0.0210 | 1.22 | 0.2165 | 1.16 | 0.6609 | 1.09 | 0.7828 | 1.07 |
| CFB | 0.8012 | 1.07 | 0.5560 | 1.18 | 0.0829 | 1.71 | 0.7846 | 0.87 | 0.5894 | 0.68 |
| CTSW | 0.9341 | 0.99 | 0.2161 | 0.84 | 0.7345 | 0.93 | 0.0459 | 0.56 | 0.0286 | 1.40 |
| FGA | 0.8311 | 1.04 | 0.6930 | 1.08 | 0.3998 | 1.28 | 0.6633 | 1.15 | 0.8176 | 0.90 |
| FGFR1 | 0.0092 | 0.64 | 0.0366 | 0.69 | 0.1384 | 0.71 | 0.3325 | 0.71 | 0.1673 | 0.56 |
| FN1 | 0.7124 | 0.96 | 0.0883 | 0.75 | 0.2792 | 0.74 | 0.1251 | 0.58 | 0.0135 | 1.58 |
| GABARA | 0.0326 | 1.35 | 0.0049 | 1.66 | 0.0112 | 1.90 | 0.6362 | 1.22 | 0.7671 | 1.14 |
| PL2 | | | | | | | | | | |
| GPHN | 0.5741 | 0.93 | 0.0867 | 0.76 | 0.8016 | 0.95 | 0.0756 | 0.60 | 0.0074 | 1.63 |
| GRP | 0.1150 | 0.91 | 0.3888 | 0.94 | 0.9402 | 1.01 | 0.4915 | 0.92 | 0.2440 | 0.84 |
| IL23A | 0.7794 | 0.96 | 0.1160 | 0.76 | 0.0775 | 0.53 | 0.6231 | 0.88 | 0.0136 | 1.47 |
| IL3 | 0.1571 | 1.15 | 0.3842 | 1.10 | 0.2913 | 1.23 | 0.3890 | 0.83 | 0.3693 | 1.28 |
| IL4R | 0.6152 | 1.11 | 0.3383 | 1.23 | 0.2050 | 1.44 | 0.4637 | 1.37 | 0.4812 | 0.68 |
| KDM4A | 0.0594 | 1.20 | 0.0698 | 1.22 | 0.3602 | 1.27 | 0.8725 | 0.97 | 0.4712 | 1.15 |
| KRT19 | 0.0293 | 0.81 | 0.0275 | 0.77 | 0.1589 | 0.74 | 0.0176 | 0.59 | 0.8006 | 0.96 |
| KRT7 | 0.0491 | 1.15 | 0.5026 | 1.06 | 0.2201 | 0.84 | 0.0396 | 1.31 | 0.0008 | 1.55 |
| L1CAM | 0.0725 | 1.19 | 0.1297 | 1.19 | 0.0689 | 1.41 | 0.9284 | 0.98 | 0.3973 | 1.20 |
| LAMC1 | 0.0242 | 1.13 | 0.0003 | 1.26 | 0.1425 | 1.15 | 0.0819 | 1.22 | 0.0763 | 0.80 |
| LEPR | 0.4214 | 1.05 | 0.3180 | 1.07 | 0.6410 | 1.05 | 0.0425 | 1.24 | 0.6934 | 0.96 |
| MAGEB4 | 0.0347 | 1.17 | 0.0022 | 1.28 | 0.0001 | 1.60 | 0.3936 | 1.11 | 0.5817 | 0.91 |
| MAGED2 | 0.0517 | 1.17 | 0.0069 | 1.24 | 0.0281 | 1.28 | 0.4067 | 1.10 | 0.0867 | 0.49 |
| MIF | 0.0195 | 0.70 | 0.1566 | 0.79 | 0.5506 | 0.89 | 0.3626 | 0.73 | 0.0320 | 0.49 |
| MITF | 0.1663 | 1.08 | 0.4909 | 1.05 | 0.2028 | 0.86 | 0.0060 | 1.28 | 0.1509 | 1.18 |
| MUM1 | 0.0259 | 0.78 | 0.0074 | 0.69 | 0.0787 | 0.69 | 0.2835 | 0.81 | 0.4382 | 1.15 |
| NCOA1 | 0.0092 | 0.85 | 0.0363 | 0.87 | 0.0166 | 0.74 | 0.7861 | 0.97 | 0.0233 | 0.68 |
| PCDH1 | 0.6480 | 0.98 | 0.7551 | 0.98 | 0.3527 | 0.92 | 0.2217 | 1.13 | 0.5825 | 0.94 |
| PIAS3 | 0.0001 | 1.29 | 0.0002 | 1.29 | 0.3222 | 1.14 | 0.0035 | 1.32 | 0.6339 | 1.09 |
| PRKCI | 0.0476 | 1.16 | 0.0454 | 1.18 | 0.3070 | 1.15 | 0.1695 | 1.16 | 0.5245 | 1.14 |
| RELT | 0.3255 | 1.06 | 0.1089 | 1.11 | 0.0555 | 1.19 | 0.4862 | 0.88 | 0.2962 | 0.85 |
| RPLP0 | 0.0200 | 1.28 | 0.2950 | 1.14 | 0.3784 | 1.15 | 0.1426 | 1.40 | 0.0073 | 1.74 |
| RPLP2 | 0.0013 | 1.37 | 0.0073 | 1.35 | 0.3761 | 1.21 | 0.0025 | 1.75 | 0.1249 | 1.40 |
| SDCBP | 0.0106 | 0.66 | 0.0624 | 0.71 | 0.0275 | 0.52 | 0.8785 | 0.97 | 0.1147 | 0.55 |
| SPA17 | 0.9909 | 1.00 | 0.2617 | 1.07 | 0.1289 | 0.85 | 0.0165 | 1.25 | 0.0026 | 0.61 |
| SPTBN1 | 0.2718 | 1.08 | 0.1657 | 1.11 | 0.7897 | 1.03 | 0.4152 | 1.11 | 0.1780 | 0.75 |
| SUMO2 | 0.0215 | 0.80 | 0.0571 | 0.81 | 0.0019 | 0.46 | 0.2611 | 0.85 | 0.1995 | 0.76 |
| TMEM98 | 0.0389 | 0.87 | 0.0147 | 0.82 | 0.0612 | 0.78 | 0.9159 | 1.01 | 0.2175 | 1.17 |
| TP53 | 0.5806 | 1.06 | 0.3057 | 0.86 | 0.9439 | 0.99 | 0.1028 | 0.60 | 0.0863 | 1.21 |
| TPM2 | 0.0648 | 1.14 | 0.0007 | 1.33 | 0.0562 | 1.29 | 0.0085 | 1.38 | 0.0612 | 0.72 |
| UBE2Z | 0.0558 | 1.25 | 0.0136 | 1.35 | 0.1463 | 1.34 | 0.5452 | 1.11 | 0.4863 | 0.74 |
| UBTF | 0.6388 | 1.07 | 0.1961 | 1.21 | 0.8329 | 0.94 | 0.1965 | 1.23 | 0.0961 | 0.47 |

### Example 18

### Development of Optimized Marker Panels for Colitis

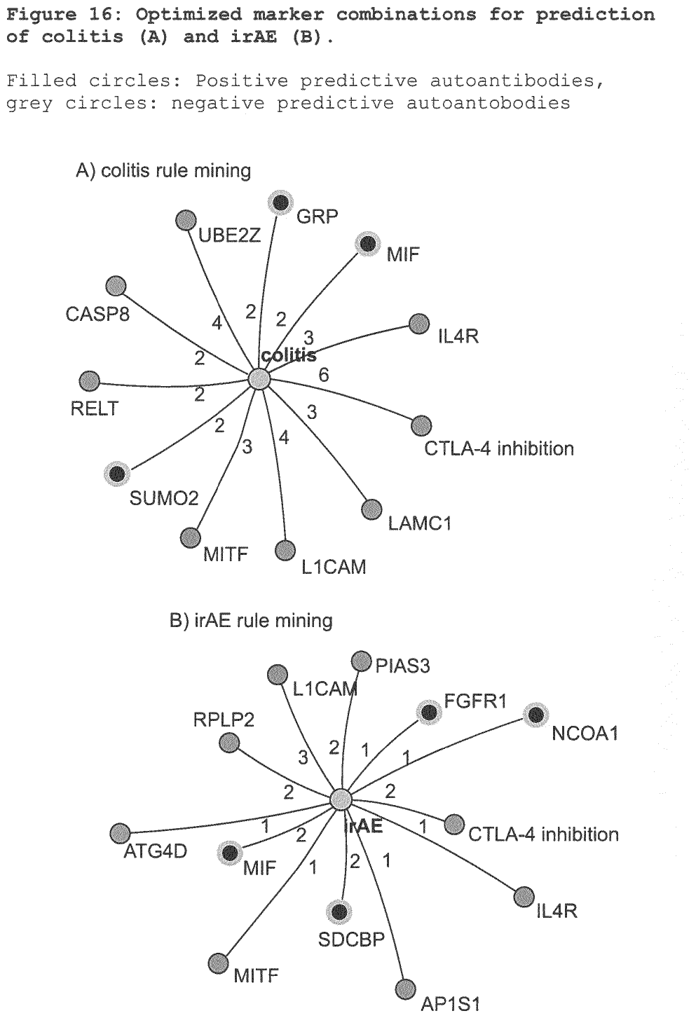

As single markers show very limited sensitivity to predict colitis, we explored association rules of markers exhibiting the highest mutual information for colitis. FIG. 16A shows a set of the best 10 markers for colitis prediction. The sets include autoantibody reactivities predicting an increased risk (RELT, CASP8, UBE2Z, IL4R, LAMC1, L1CAM, MITF) but also a reduced risk (SUMO2, GRP, MIF) to develop colitis.

### Example 19

### Development of Optimized Marker Panels for irAE

As single markers show very limited sensitivity to predict irAE, we explored association rules of markers exhibiting the highest mutual information for irAE. FIG. 16B shows a set of the best autoantibody markers for irAE prediction. The sets include autoantibody specificities predicting an increased risk (IL4R, L1CAM, MITF, PIAS3, AP1S1, ATG4D, RPLP2) but also a predicting reduced risk (MIF, NCOA1, FGFR1, SDCBP) to develop an irAE.

### REFERENCES

Bei, R., Masuelli, L., Palumbo, C., Modesti, M., and Modesti, A. (2009). A common repertoire of autoantibodies is shared by cancer and autoimmune disease patients: Inflammation in their induction and impact on tumor growth. Cancer Lett. 281, 8-23.

Bresnick, A. R., Weber, D. J., and Zimmer, D. B. (2015). S100 proteins in cancer. Nat. Rev. Cancer 15, 96-109.

Buchbinder, E. I., and Desai, A. (2016). CTLA-4 and PD-1 Pathways: Similarities, Differences, and Implications of Their Inhibition. Am. J. Clin. Oncol. 39, 98-106.

Budde, P., Zucht, H.-D., Vordenbäumen, S., Goehler, H., Fischer-Betz, R., Gamer, M., Marquart, K., Rengers, P., Richter, J., Lueking, A., et al. (2016). Multiparametric detection of autoantibodies in systemic lupus erythematosus. Lupus 25, 812-822.

Callahan, M. K., Postow, M. A., and Wolchok, J. D. (2013). Immunomodulatory therapy for melanoma: Ipilimumab and beyond. Clin. Dermatol. 31, 191-199.

Chiaruttini, G., Mele, S., Opzoomer, J., Crescioli, S., Ilieva, K. M., Lacy, K. E., and Karagiannis, S. N. (2017). B cells and the humoral response in melanoma: The overlooked players of the tumor microenvironment. OncoImmunology 6, e1294296.

Cuda, C. M., Pope, R. M., and Perlman, H. (2016). The inflammatory role of phagocyte apoptotic pathways in rheumatic diseases. Nat. Rev. Rheumatol. 12, 543-558.

Dai, N., Ji, F., Wright, J., Minichiello, L., Sadreyev, R., and Avruch, J. (2017). IGF2 mRNA binding protein-2 is a tumor promoter that drives cancer proliferation through its client mRNAs IGF2 and HMGA1. ELife 6.

Das, S. K., Bhutia, S. K., Kegelman, T. P., Peachy, L., Oyesanya, R. A., Dasgupta, S., Sokhi, U. K., Azab, B., Dash, R., Quinn, B. A., et al. (2012). MDA-9/syntenin: a positive gatekeeper of melanoma metastasis. Front. Biosci. Landmark Ed. 17, 1-15.

Eisenhauer, E. A., Therasse, P., Bogaerts, J., Schwartz, L. H., Sargent, D., Ford, R., Dancey, J., Arbuck, S., Gwyther, S., Mooney, M., et al. (2009). New response evaluation criteria in solid tumours: Revised RECIST guideline (version 1.1). Eur. J. Cancer 45, 228-247.

Ferrer, C. M., Sodi, V. L., and Reginato, M. J. (2016). O-GlcNAcylation in Cancer Biology: Linking Metabolism and Signaling. J. Mol. Biol. 428, 3282-3294.

Garg, H., Suri, P., Gupta, J. C., Talwar, G. P., and Dubey, S. (2016). Survivin: a unique target for tumor therapy. Cancer Cell Int. 16.

Gulley, J. L., Madan, R. A., Tsang, K. Y., Jochems, C., Marté, J. L., Farsaci, B., Tucker, J. A., Hodge, J. W., Liewehr, D. J., Steinberg, S. M., et al. (2014). Immune Impact Induced by PROSTVAC (PSA-TRICOM), a Therapeutic Vaccine for Melanoma. Cancer Immunol. Res. 2, 133-141.

Hodi, F. S., Chesney, J., Pavlick, A. C., Robert, C., Grossmann, K. F., McDermott, D. F., Linette, G. P., Meyer, N., Giguere, J. K., Agarwala, S. S., et al. (2016). Combined nivolumab and ipilimumab versus ipilimumab alone in patients with advanced melanoma: 2-year overall survival outcomes in a multicentre, randomised, controlled, phase 2 trial. Lancet Oncol. 17, 1558-1568.

Houles, T., and Roux, P. P. (2017). Defining the role of the RSK isoforms in cancer. Semin. Cancer Biol.

Kindt, N., Journe, F., Laurent, G., and Saussez, S. (2016). Involvement of macrophage migration inhibitory factor in cancer and novel therapeutic targets. Oncol. Lett. 12, 2247-2253.

Larkin, J., Chiarion-Sileni, V., Gonzalez, R., Grob, J. J., Cowey, C. L., Lao, C. D., Schadendorf, D., Dummer, R., Smylie, M., Rutkowski, P., et al. (2015). Combined Nivolumab and Ipilimumab or Monotherapy in Untreated Melanoma. N. Engl. J. Med. 373,23-34.

Luo, L. Y., and Hahn, W. C. (2015). Oncogenic Signaling Adaptor Proteins. J. Genet. Genomics 42, 521-529.

Manson, G., Norwood, J., Marabelle, A., Kohrt, H., and Houot, R. (2016). Biomarkers associated with checkpoint inhibitors. Ann. Oncol. 27, 1199-1206.

Moscat, J., Karin, M., and Diaz-Meco, M. T. (2016). p62 in Cancer: Signaling Adaptor Beyond Autophagy. Cell 167, 606-609.

Peng, S., Wang, K., Gu, Y., Chen, Y., Nan, X., Xing, J., Cui, Q., Chen, Y., Ge, Q., and Zhao, H. (2015). TRAF3IP3, a novel autophagy up-regulated gene, is involved in marginal zone B lymphocyte development and survival. Clin. Exp. Immunol. 182, 57-68.

Robin, X., Turck, N., Hainard, A., Tiberti, N., Lisacek, F., Sanchez, J.-C., and Müller, M. (2011). pROC: an open-source package for R and S+ to analyze and compare ROC curves. BMC Bioinformatics 12, 77.

Shi, Y., Feng, Y., Kang, J., Liu, C., Li, Z., Li, D., Cao, W., Qiu, J., Guo, Z., Bi, E., et al. (2007). Critical regulation of CD4+ T cell survival and autoimmunity by beta-arrestin 1. Nat. Immunol. 8, 817-824.

Smolinsky, B., Eichler, S. A., Buchmeier, S., Meier, J. C., and Schwarz, G. (2008). Splice-specific Functions of Gephyrin in Molybdenum Cofactor Biosynthesis. J. Biol. Chem. 283, 17370-17379.

Spain, L., Diem, S., and Larkin, J. (2016). Management of toxicities of immune checkpoint inhibitors. Cancer Treat. Rev. 44, 51-60.

Topalian, S. L., Taube, J. M., Anders, R. A., and Pardoll, D. M. (2016). Mechanism-driven biomarkers to guide immune checkpoint blockade in cancer therapy. Nat. Rev. Cancer 16, 275-287.

Tusher, V. G., Tibshirani, R., and Chu, G. (2001). Significance analysis of microarrays applied to the ionizing radiation response. Proc. Natl. Acad. Sci. U.S.A. 98, 5116-5121.

Uehara, T., Kage-Nakadai, E., Yoshina, S., Imae, R., and Mitani, S. (2015). The Tumor Suppressor BCL7B Functions in the Wnt Signaling Pathway. PLoS Genet. 11, e1004921.

The present invention is not to be limited in scope by the specific embodiments described herein.

Indeed, various modifications of the invention in addition to those described herein will become apparent to those skilled in the art from the foregoing description and accompanying figures.

Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

1. A method of selecting a melanoma patient for treatment with one or more checkpoint inhibitors, the method comprising:
(a) determining in a sample obtained from the patient the level(s) of autoantibodies specifically binding to one or more of the antigens selected from the following:
ACTB, AMPH, AQP4, BAG6, BICD2, BIRC5, C15orf48, C17orf85, CALR, CCNB1, CENPH, CENPV, CEP131, CTAG1B, CTSW, EIF3E, EOMES, FGFR1, FLNA, FRS2, GNAI2, GPHN, GRP, GSK3A, HES1, IGF2BP2, IL23A, IL36RN, KRT19, MAZ, MIF, MLLT6, MUM1, NCOA1, NOVA2, NRIP1, PAPOLG, PPP1R2, PTPRR, RALY, SDCBP, SIVA1, SNRNP70, SNRPA, SNRPD1, SPA17, SSB, SUM02, TEX264, TMEM98, TRAF3IP3, XRCC5 and XRCC6; and
(b) comparing the level(s) of autoantibodies determined in (a) with a predetermined cut-off value for autoantibodies specifically binding to the same one or more antigens,
wherein if the level of autoantibodies determined in the patient sample is higher than the predetermined cut-off value, the patient is selected for treatment with the checkpoint inhibitor(s).

2. The method of claim 1, wherein the one or more antigens are selected from the following:
SIVA1, IGF2BP2, AQP4, C15orf48, CTAG1B and PAPOLG.

3. The method of claim 1, wherein the one or more antigens are selected from the following:
FRS2, BIRC5, EIF3E, CENPH and PAPOLG.

4. The method of claim 1, wherein the one or more antigens are selected from the following:
NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2 and IL36RN.

5. The method of claim 1, wherein the one or more antigens are selected from the following:
SUM02, GRP, SDCBP, AMPH, IL23A, GPHN, BAG6, BICD2, TMEM98, MUM1, CTSW, NCOA1, MIF, SPA17, FGFR1 and KRT19.

6. A method of selecting a melanoma patient for treatment with one or more checkpoint inhibitors, the method comprising:
(a) determining in a sample obtained from the patient the level(s) of autoantibodies specifically binding to one or more of the antigens selected from the following:
ABCB8, AKT2, AMPH, AP1S1, AP2B1, ATG4D, ATP13A2, BTBD2, BTRC, CAP2, CASP10, CASP8, CFB, CREB3L1, CTSW, EGFR, EIF4E2, ELMO2, EOMES, ERBB3, FADD, FGA, FN1, FOXO1, FRS2, GABARAPL2, HSPA1B, HSPB1, IL23A, IL3, IL4R, KDM4A, KLKB1, KRT7, L1CAM, LAMB2, LAMC1, LEPR, LGALS3BP, MAGEB4, MAGED2, MAPT, MITF, MUC12, MUM1, OGT, PCDH1, PDCD6IP, PECAM1, PIAS3, PLIN2, PPL, PPP1R12A, PRKCI, RAPGEF3, RELT, RPLP0, RPLP2, SIGIRR, SIPA1L1, SPA17, SPTB, SPTBN1, SUFU, TEX264, TMEM98, TOLLIP, TONSL, TP53, TPM2, TRIP4, UBAP1, UBE2Z, UBTF, XRCC5 and XRCC6;
and
(b) comparing the level(s) of autoantibodies determined in (a) with a predetermined cut-off value for autoantibodies specifically binding to the same one or more antigens,
wherein if the level of autoantibodies determined in the patient sample is not higher than the predetermined cut-off value, the patient is selected for treatment with the checkpoint inhibitor(s).

7. The method of claim 6, wherein the one or more antigens are selected from the following:
TEX264, CREB3L1, HSPA1B, SPTB, MUC12, ERBB3, CASP10, FOXO1, FRS2, PPP1R12A and CAP2.

8. The method of claim 6, wherein the one or more antigens are selected from the following:
EOMES, CREB3L1, FRS2, PLIN2, SIPA1L1, ABCB8, MAPT, XRCC5, XRCC6, UBAP1, TRIP4 and EIF4E2.

9. The method of claim 6, wherein the one or more antigens are selected from the following:
FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

10. The method of claim 6, wherein the one or more antigens are selected from the following:
MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17.

11. The method of any one of claims 6-10, additionally comprising the steps of the methods according to any one of claims 1-5.

12. A method of selecting a melanoma patient for treatment with one or more checkpoint inhibitors, the method comprising:
(a) determining in a sample obtained from the patient the level(s) of autoantibodies specifically binding to one or more of the antigens selected from the following:
ARRB1, BCL7B, CCDC51, CEACAM5, CSNK2A1, DFFA, DHFR, FGFR1, GNG12, GRAMD4, GRK6, HDAC1, LAMC1, MSH2, MIF, MMP3, RPS6KA1, S100A8, S100A14, SHC1 and USB1;
and
(b) comparing the level(s) of autoantibodies determined in (a) with a predetermined cut-off value for autoantibodies specifically binding to the same one or more antigens,
wherein if the level of autoantibodies determined in the patient sample is lower than the pre-determined cut-off value, the patient is selected for treatment with the checkpoint inhibitor(s).

13. The method of claim 12, wherein the one or more antigens are selected from the following:
GRK6, MIF, FGFR1 and GRAMD4.

14. The method of claim 12, wherein the one or more antigens are selected from the following:
GNG12, CCDC51, USB1, GRAMD4, RPS6KA1 and BCL7B.

15. The method of claim 12, wherein the one or more antigens are selected from the following:
S100A14, MMP3, SHC1, CSNK2A1, DFFA, S100A8, HDAC1, MSH2, CEACAM5, DHFR, LAMC1 and ARRB1.

16. The method of any one of claims 12-15, additionally comprising the steps of the methods according to any one of claims 1-11.

17. A method of selecting a melanoma patient for treatment with one or more checkpoint inhibitors, the method comprising:
(a) determining in a sample obtained from the patient the level(s) of autoantibodies specifically binding to one or more of the antigens selected from the following:
CXXC1, EGLN2, ELMO2, HIST2H2AA3, HSPA2, HSPD1, IL17A, LARP1, POLR3B, RFWD2, RPRM, S100A8, SMAD9, SQSTM1 and WHSC1L1; and
(b) comparing the level(s) of autoantibodies determined in (a) with a predetermined cut-off value for autoantibodies specifically binding to the same one or more antigens,
wherein if the level of autoantibodies determined in the patient sample is not lower than the pre-determined cut-off value, the patient is selected for treatment with the checkpoint inhibitor(s).

18. The method of claim 17, wherein the one or more antigens are selected from the following:
HSPA2, SMAD9, HIST2H2AA3 and S100A8.

19. The method of claim 17, wherein the one or more antigens are selected from the following:
POLR3B, ELMO2, RFWD2, SQSTM1, HSPD1 and IL17A.

20. The method of claim 17, wherein the one or more antigens are selected from the following:
CXXC1, LARP1, EGLN2, RPRM, WHSC1L1 and S100A8.

21. The method of any one of claims 17-20, additionally comprising the steps of the methods according to any one of claims 1-16.

22. The method of any one of claims 1-21, wherein the one or more checkpoint inhibitors are selected from CTLA-4 inhibitors, PD-1 inhibitors and PD-L1 inhibitors.

23. The method of claim 22, wherein the checkpoint inhibitor is ipilimumab.

24. The method of claim 22, wherein the checkpoint inhibitor is nivolumab.

25. The method of claim 22, wherein the checkpoint inhibitor is pembrolizumab.

26. The method of claim 22, wherein the checkpoint inhibitors are a combination of ipilimumab and nivolumab.

27. The method of any one of claims 1-26, wherein the level of autoantibodies in the patient sample is determined by contacting the sample with antigen immobilized onto a solid support.

28. The method of any one of claims 1-27, wherein the levels of autoantibodies specifically binding to two or more, three or more, four or more, five or more antigens are determined in the patient sample.

29. The method of claim 28, wherein the levels of autoantibodies in the patient sample are determined by contacting the sample with a panel or array of the antigens immobilized onto a solid support.

30. The method of any one of claims 1-29, wherein the predetermined cut-off value for autoantibodies is the average level of autoantibodies specifically binding to the antigen determined for a control cohort of melanoma patients.

31. The method of any one of claims 1-30, further comprising administering the one or more checkpoint inhibitors to the patient.

32. A method of treating melanoma in a subject, the method comprising administering to the subject one or more checkpoint inhibitors, wherein the subject is selected for treatment in accordance with the methods of any one of claims 1-31.

33. A method of predicting a melanoma patient's responsiveness to treatment with a checkpoint inhibitor, the method comprising:
(a) determining in a sample obtained from the patient the level(s) of autoantibodies specifically binding to one or more of the antigens selected from the following:
ACTB, AQP4, BIRC5, C15orf48, C17orf85, CALR, CCNB1, CENPH, CENPV, CEP131, CTAG1B, EIF3B, EOMES, FLNA, FRS2, GNAI2, GPHN, GSK3A, HES1, IGF2BP2, IL36RN, MAZ, MLLT6, NOVA2, NRIP1, PAPOLG, PPP1R2, PTPRR, RALY, SIVA1, SNRNP70, SNRPA, SNRPD1, SSB, TEX264, TRAF3IP3, XRCC5 and XRCC6; and
(b) comparing the level(s) of autoantibodies determined in (a) with a predetermined cut-off value for autoantibodies specifically binding to the same one or more antigens,
wherein if the level of autoantibodies determined in the patient sample is higher than the predetermined cut-off value, improved responsiveness is predicted.

34. The method of claim 33, wherein the checkpoint inhibitor is ipilimumab and the one or more antigens are selected from the following: FRS2, GPHN, BIRC5, EIF3E, CENPH and PAPOLG.

35. The method of claim 33, wherein the checkpoint inhibitor is pembrolizumab and the one or more antigens are selected from the following: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264 SNRNP70, CEP131, SNRPA, CENPV, NRIP1, CCNB1, RALY, CALR, GNAI2, IL36RN, FGA and GHPN.

36. A method of predicting a melanoma patient's responsiveness to treatment with a checkpoint inhibitor, the method comprising:
(a) determining in a sample obtained from the patient the level(s) of autoantibodies specifically binding to one or more of the antigens selected from the following:
GRK6, MIF, FGFR1 GRAMD4, GNG12, CCDC51, USB1, RPS6KA1, BCL7B, S100A14, MMP3, SHC1, CSNK2A1, DFFA, S100A8, HDAC1, MSH2, CEACAM5, DHFR, LAMC1 and ARRB1; and
(b) comparing the level of autoantibodies determined in (a) with a predetermined cut-off value for autoantibodies specifically binding to the same one or more antigens,
wherein if the level of autoantibodies determined in the patient sample is lower than the predetermined cut-off value, improved responsiveness is predicted.

37. The method of claim 36, wherein the checkpoint inhibitor is ipilimumab and the one or more antigens are selected from the following: GNG12, CCDC51, USB1, GRAMD4, RPS6KA1, BCL7B.

38. The method of claim 36, wherein the checkpoint inhibitor is pembrolizumab and the one or more antigens are selected from the following: S100A14, MMP3, SHC1, CSNK2A1, DFFA, S100A8, HDAC1, MSH2, CEACAM5, DHFR, LAMC1 and ARRB1.

39. The method of any one of claims 36-38, wherein the method additionally comprises the steps of the methods according to any one of claims 33-35.

40. The method of any one of claims 33-39, wherein responsiveness to treatment is assessed by measuring complete response (CR), partial response (PR) or stable disease (SD).

41. A method of predicting survival in a melanoma patient responsive to treatment with a checkpoint inhibitor, the method comprising:
(a) determining in a sample obtained from the patient the level(s) of autoantibodies specifically binding to one or more of the antigens selected from the following:
ACTB, AQP4, BIRC5, C15orf48, C17orf85, CALR, CCNB1, CENPH, CENPV, CEP131, CTAG1B, EIF3B, EOMES, FLNA, FRS2, GNAI2, GPHN, GSK3A, HES1, IGF2BP2, IL36RN, MAZ, MLLT6, NOVA2, NRIP1, PAPOLG, PPP1R2, PTPRR, RALY, SIVA1, SNRNP70, SNRPA, SNRPD1, SSB, TEX264, TRAF3IP3, XRCC5 and XRCC6; and
(b) comparing the level(s) of autoantibodies determined in (a) with a predetermined cut-off value for autoantibodies specifically binding to the same one or more antigens,
wherein if the level of autoantibodies determined in the patient sample is higher than the predetermined cut-off value, improved survival is predicted.

42. The method of claim 41, wherein the checkpoint inhibitor is ipilimumab and the one or more antigens are selected from the following: FRS2, GPHN, BIRC5, EIF3E, CENPH and PAPOLG.

43. The method of claim 41, wherein the checkpoint inhibitor is pembrolizumab and the one or more antigens are selected from the following: NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264 SNRNP70, CEP131, SNRPA, CENPV, NRIP1, CCNB1, RALY, CALR, GNAI2 IL36RN, FGA and GHPN.

44. A method of predicting survival in a melanoma patient responsive to treatment with a checkpoint inhibitor, the method comprising:
(a) determining in a sample obtained from the patient the level(s) of autoantibodies specifically binding to one or more of the antigens selected from the following:
GRK6, MIF, FGFR1 GRAMD4, GNG12, CCDC51, USB1, GRAMD4, RPS6KA1, BCL7B, S100A14, MMP3, SHC1, CSNK2A1, DFFA, S100A8, HDAC1, MSH2, CEACAM5, DHFR, LAMC1 and ARRB1; and
(b) comparing the level of autoantibodies determined in (a) with a predetermined cut-off value for autoantibodies specifically binding to the same one or more antigens,
wherein if the level of autoantibodies determined in the patient sample is lower than the predetermined cut-off value, improved survival is predicted.

45. The method of claim 44, wherein the checkpoint inhibitor is ipilimumab and the one or more antigens are selected from the following: GNG12, CCDC51, USB1, GRAMD4, RPS6KA1 and BCL7B.

46. The method of claim 44, wherein the checkpoint inhibitor is pembrolizumab and the one or more antigens are selected from the following: S100A14, MMP3, SHC1, CSNK2A1, DFFA, S100A8, HDAC1, MSH2, CEACAM5, DHFR, LAMC1 and ARRB1.

47. The method of any one of claims 44-46, wherein the method additionally comprises the steps of the methods according to any one of claims 41-43.

48. The method of any one of claims 41-47, wherein survival is overall survival or progression-free survival.

49. A method of predicting the risk of immune-related adverse events (irAEs) in a melanoma patient treated with one or more checkpoint inhibitors, the method comprising:
(a) determining in a sample obtained from the patient the level(s) of autoantibodies specifically binding to one or more of the antigens selected from the following:
TEX264, CREB3L1, HSPA1B, SPTB, MUC12, ERBB3, CASP10, FOXO1, FRS2, PPP1R12A, CAP2, EOMES, CREB3L1, PLIN2, SIPA1L1, ABCB8, MAPT, XRCC5, XRCC6, UBAP1, TRIP4, EIF4E2, FADD, OGT, HSPB1, ATP13A2, SIGIRR, HSPA1B, SPTB, PDCD6IP, RAPGEF3, PECAM1, PPL, TONSL, ELMO2, LAMB2, BTRC, SUFU, LGALS3BP, KLKB1, EGFR, TOLLIP, MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17; and
(b) comparing the level of autoantibodies determined in (a) with a predetermined cut-off value for autoantibodies specifically binding to the same one or more antigens,
wherein if the level of autoantibodies determined in the patient sample is higher than the predetermined cut-off value, the patient is determined to be at higher risk of irAEs.

50. The method of claim 49, wherein the one or more antigens are selected from the following:
TEX264, CREB3L1, HSPA1B, SPTB, MUC12, ERBB3, CASP10, FOXO1, FRS2, PPP1R12A and CAP2.

51. The method of claim 49, wherein the one or more antigens are selected from the following:
EOMES, CREB3L1, FRS2, PLIN2, SIPA1L1, ABCB8, MAPT, XRCC5, XRCC6, UBAP1, TRIP4 and EIF4E2.

52. The method of claim 49, wherein the one or more antigens are selected from the following:
FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

53. The method of claim 49, wherein the one or more antigens are selected from the following:
MAGED2, PIAS3, MITF, AP2B1, PRKCI, AKT2, BTBD2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17.

54. The method of claim 53, wherein the irAE is colitis and the one or more antigens are selected from: MAGED2, PIAS3, MITF, AP2B1, PRKCI, AKT2, UBE2Z, L1CAM, GABARAPL2, LAMC1, RPLP0, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, FN1, BTBD2, MAGEB4, CTSW and MUM1.

55. The method of claim 53, wherein the one or more antigens are selected from IL4R, L1CAM, MITF, PIAS3, AP1S1, ATG4D and RPLP2.

56. The method of claim 53 or claim 54, wherein the one or more antigens are selected from:
RELT, CASP8, UBE2Z, IL4R, LAMC1, L1CAM and MITF.

57. The method of claim 53 or claim 54, wherein the one or more antigens are selected from:
PIAS3, RPLP2, ATG4D, KRT7, TPM2, GABARAPL2 and MAGEB4.

58. The method of claim 53 or claim 54, wherein the one or more antigens are selected from MAGED2, PIAS3, MITF, AP2B1 and PRKCI.

59. The method of claim 53 or claim 54, wherein the antigen is MAGED2.

60. The method of claim 53, wherein the antigen is KRT7.

61. The method of claim 53 or claim 54, wherein the checkpoint inhibitor is ipilimumab and the one or more antigens are selected from: UBE2Z, L1CAM, GABARAPL2, CFB, IL3, RELT, FGA, and IL4R.

62. The method of claim 53, wherein the checkpoint inhibitors are ipilimumab and nivolumab and the one or more antigens are selected from PIAS3, MITF, PRKCI, AP2B1, PDCH1, SPTBN1 and UBTF.

63. A method of predicting the risk of immune-related adverse events (irAEs) in a melanoma patient treated with one or more checkpoint inhibitors, the method comprising:
(a) determining in a sample obtained from the patient the level(s) of autoantibodies specifically binding to one or more of the antigens selected from the following:
SUM02, GRP, SDCBP, AMPH, GPHN, BAG6, BICD2, TMEM98, MUM1, CTSW, NCOA1, MIF, SPA17, FGFR1 and KRT19; and
(ii) comparing the level of autoantibodies determined in (a) with a predetermined cut-off value for autoantibodies specifically binding to the same one or more antigens,
wherein if the level of autoantibodies determined in the patient sample is higher than the predetermined cut-off value, the patient is determined to be at lower risk of irAEs.

64. The method of claim 63, wherein the one or more antigens are selected from the following:
NCOA1, MIF, SDCB4, MUM1, FGFR1 and KRT19.

65. The method of claim 63, wherein the one or more antigens are selected from the following:
MIF, NCOA1, FGFR1 and SDCBP.

66. The method of claim 63, wherein the one or more antigens are selected from SUMO2, GRP and MIF.

67. The method of claim 63, wherein the irAE is colitis and the one or more antigens are selected from the following: SUM02, GRP, SDCBP, GPHN, BAG6, BICD2 and TMEM98.

68. The method of any one of claims 63-67, wherein the method additionally comprises the steps of the method of any one of claims 49-62.

69. A method of predicting the risk of immune-related adverse events (irAEs) in a melanoma patient treated with one or more checkpoint inhibitors, the method comprising:
(a) determining in a sample obtained from the patient the level(s) of autoantibodies specifically binding to one or more of the antigens selected from the following:
CXXC1, EGLN2, ELMO2, HIST2H2AA3, HSPA2, HSPD1, IL17A, LARP1, POLR3B, RFWD2, RPRM, S100A8, SMAD9, SQSTM1, and WHSC1L1; and
(b) comparing the level of autoantibodies determined in (a) with a predetermined cut-off value for autoantibodies specifically binding to the same one or more antigens, wherein if the level of autoantibodies determined in the patient sample is lower than the predetermined cut-off value, the patient is determined to be at higher risk of irAEs.

70. The method of claim 69, wherein the one or more antigens are selected from the following:
HSPA2, SMAD9, HIST2H2AA3 and S100A8.

71. The method of claim 69, wherein the one or more antigens are selected from the following:
POLR3B, ELMO2, RFWD2, SQSTM1, HSPD1 and IL17A.

72. The method of claim 69, wherein the one or more antigens are selected from the following:
CXXC1, LARP1, EGLN2, RPRM, WHSC1L1 and S100A8.

73. The method of any one of claims 69-72, wherein the method additionally comprises the steps of the method of any one of claims 49-68.

74. The method of any one of claims 33-73, wherein the checkpoint inhibitors are selected from CTLA-4 inhibitors, PD-1 inhibitors and PD-L1 inhibitors.

75. The method of claim 74, wherein the checkpoint inhibitor is ipilimumab.

76. The method of claim 74, wherein the checkpoint inhibitor is nivolumab.

77. The method of claim 74, wherein the checkpoint inhibitor is pembrolizumab.

78. The method of claim 74, wherein the checkpoint inhibitors are a combination of ipilimumab and nivolumab

79. The method of any one of claims 33-78, wherein the level of autoantibodies in the patient sample is determined by contacting the sample with antigen immobilized onto a solid support.

80. The method of any one of claims 33-79, wherein the levels of autoantibodies specifically binding to two or more, three or more, four or more, five or more antigens are determined in the patient sample.

81. The method of claim 80, wherein the levels of autoantibodies in the patient sample are determined by contacting the sample with a panel or array of the antigens immobilized onto a solid support.

82. The method of any one of claims 33-81, wherein the predetermined cut-off value for autoantibodies is the average level of autoantibodies specifically binding to the antigen determined for a control cohort of melanoma patients.

83. A method of detecting melanoma in a mammalian subject by detecting an autoantibody in a sample obtained from the mammalian subject,
wherein the autoantibody specifically binds to an antigen selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4 and NOVA2, and
wherein the presence of autoantibodies at a level above a pre-determined cut-off value is indicative of melanoma;
and/or
wherein the autoantibody specifically binds to an antigen selected from: SNRPA, NRIP1, UBAP1, TEX264, PLIN2, LAMC1, CENPH, USB1, ABCB8, C15orf48/NMES1 and MAGED1, and wherein the presence of autoantibodies at a level below a pre-determined cut-off value is indicative of melanoma.

84. A method of diagnosing melanoma in a mammalian subject by detecting an autoantibody in a sample obtained from the mammalian subject,
wherein the autoantibody specifically binds to an antigen selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4 and NOVA2, and
wherein the subject is diagnosed as having melanoma if the presence of autoantibodies is at a level above a pre-determined cut-off value;
and/or
wherein the autoantibody specifically binds to an antigen selected from: SNRPA, NRIP1, UBAP1, TEX264, PLIN2, LAMC1, CENPH, USB1, ABCB8, C15orf48/NMES1 and MAGED1, and wherein the subject is diagnosed as having melanoma if the presence of autoantibodies is at a level below a pre-determined cut-off value.

85. The method of claim 83 or claim 84, wherein the method comprises:
(a) contacting the sample with the melanoma antigen; and
(b) determining the presence of complexes of the melanoma antigen bound to autoantibodies so as to determine the level of autoantibodies in the sample; and
(c) comparing the level of autoantibodies in the sample with a pre-determined cut-off value.

86. The method of any one of claims 83-85, wherein the pre-determined cut-off value is based upon a healthy cohort of mammalian subjects.

87. The method of any one of claims 83-86, wherein the autoantibody specifically binds to an antigen selected from: CREB3L1, CXCL5 and NME1.

88. The method of any one of claims 83-87, wherein autoantibodies specifically binding to two or more antigens selected from: RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4 and NOVA2 are detected.

89. The method of claim 88, wherein autoantibodies specifically binding to CREB3L1, CXCL5 and NME1 are detected.

90. The method of any one of claims 83-89, wherein autoantibodies specifically binding to two or more antigens selected from: SNRPA, NRIP1, UBAP1, TEX264, PLIN2, LAMC1, CENPH, USB1, ABCB8, C15orf48/NMES1 and MAGED1 are detected.

91. The method of any one of claims 88-90, wherein the method comprises:
(a) contacting the sample with a panel of two or more antigens;
(b) determining the presence of autoantibody-antigen complexes for each of the antigens so as to determine the level of autoantibodies specifically binding each antigen in the sample; and
(c) comparing the levels of autoantibodies for each antigen with pre-determined cut-off values.

92. The method of claim 91, wherein if the level of autoantibodies specifically binding to one or more of the antigens is above or below the pre-determined cut-off value, the result is indicative of melanoma or a positive melanoma diagnosis.

93. The method of claim 91, wherein if the level of autoantibodies specifically binding to each of the antigens tested is above or below the predetermined cut-off value, the result is indicative of melanoma or a positive melanoma diagnosis.

94. The method of any one of claims 83-93, wherein the mammalian subject is a human.

95. A kit suitable for performing the method of any one of the preceding claims, wherein the kit comprises:
(a) one or more melanoma antigens; and
(b) a reagent capable of detecting complexes of the melanoma antigen bound to autoantibodies present in the sample obtained from the melanoma patient or mammalian subject.

96. A kit for the detection of autoantibodies in a test sample obtained from a mammalian subject, the kit comprising:
(a) one or more melanoma antigens selected from the following:
RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, NOVA2, SNRPA, NRIP1, UBAP1, TEX264, PLIN2, LAMC1, CENPH, USB1, ABCB8, C15orf48/NMES1 and MAGED1; and
(b) a reagent capable of detecting complexes of the melanoma antigen bound to autoantibodies present in the test sample obtained from the mammalian subject.

97. A kit for the detection of autoantibodies in a test sample obtained from a melanoma patient, the kit comprising:
(a) one or more melanoma antigens selected from the following:
ABCB8, ACTB, AKT2, AMPH, AP1S1, AP2B1, AQP4, ARRB1, ATG4D, ATP13A2, BAG6, BCL7B, BICD2, BIRC5, BTBD2, BTRC, C15orf48, C17orf85, CALR, CAP2, CASP10, CASP8, CCDC51, CCNB1, CEACAM5, CENPH, CENPV, CEP131, CFB, CREB3L1, CSNK2A1, CTAG1B, CTSW, CXXC1, DFFA, DHFR, EGFR, EGLN2, EIF4E2, ELMO2, EOMES, ERBB3, FADD, FGA, FGFR1, FLNA, FN1, FOXO1, FRS2, GABARAPL2, GNAI2, GNG12, GPHN, GRAMD4, GRK6, GRP, GSK3A, HDAC1, HES1, HIST2H2AA3, HSPA1B, HSPA2, HSPB1, HSPD1, IGF2BP2, IL3, IL4R, IL17A, IL23A, IL36RN, KDM4A, KLKB1, KRT7, KRT19, L1CAM, LAMB2, LAMC1, LARP1, LEPR, LGALS3BP, MAGEB4, MAGED2, MAPT, MAZ, MIF, MITF, MLLT6, MMP3, MSH2, MUM1, MUC12, NCOA1, NOVA2, NRIP1, OGT, PAPOLG, PCDH1, PDCD6IP, PECAM1, PIAS3, PLIN2, POLR3B, PPL, PPP1R12A, PPP1R2, PRKCI, PTPRR, RALY, RAPGEF3, RELT, RFWD2, RPLP0, RPLP2, RPRM, RPS6KA1, S100A8, S100A14, SDCBP, SHC1, SIGIRR, SIPA1L1, SIVA1, SMAD9, SNRNP70, SNRPA, SNRPD1, SQSTM1, SPA17, SPTB, SPTBN1, SSB, SUFU, SUM02, TEX264, TMEM98, TOLLIP, TONSL, TP53, TPM2, TRAF3IP3, TRIP4, UBAP1, UBE2Z, UBTF, USB1, WHSC1L1, XRCC5 and XRCC6;
and
(b) a reagent capable of detecting complexes of the melanoma antigen bound to autoantibodies present in the test sample obtained from the melanoma patient.

98. A kit for the detection of autoantibodies in a test sample obtained from a melanoma patient, the kit comprising:
(a) one or more melanoma antigens selected from the following:
ABCB8, ACTB, AQP4, ARRB1, ATP13A2, BCL7B, BIRC5, BTRC, C15orf48, C17orf85, CALR, CAP2, CASP10, CCDC51, CCNB1, CEACAM5, CENPH, CENPV, CEP131, CREB3L1, CSNK2A1, CTAG1B, CXXC1, DFFA, DHFR, EGFR, EGLN2, EIF4E2, ELMO2, EOMES, ERBB3, FADD, FLNA, FOXO1, FRS2, GNAI2, GNG12, GRAMD4, GRK6, GSK3A, HDAC1, HES1, HIST2H2AA3, HSPA1B, HSPA2, HSPB1, HSPD1, IGF2BP2, IL17A, IL36RN, KLKB1, LAMB2, LARP1, LGALS3BP, MAPT, MAZ, MLLT6, MMP3, MSH2, MUC12, NOVA2, NRIP1, OGT, PAPOLG, PDCD6IP, PECAM1, PLIN2, POLR3B, PPL, PPP1R12A, PPP1R2, PTPRR, RALY, RAPGEF3, RFWD2, RPRM, RPS6KA1, S100A8, S100A14, SHC1, SIGIRR, SIPA1L1, SIVA1, SMAD9, SNRNP70, SNRPA, SNRPD1, SQSTM1, SPTB, SSB, SUFU, TEX264, TOLLIP, TONSL, TRAF3IP3, TRIP4, UBAP1, USB1, WHSC1L1, XRCC5 and XRCC6;
and
(b) a reagent capable of detecting complexes of the melanoma antigen bound to autoantibodies present in the test sample obtained from the melanoma patient.

99. The kit of any one of claims 95-98, further comprising:
(c) means for contacting the melanoma antigen with the test sample obtained from the mammalian subject or melanoma patient.

100. The kit of claim 99, wherein the means for contacting the melanoma antigen with the test sample comprises the antigen immobilised on a chip, slide, plate, wells of a microtitre plate, bead, membrane or nanoparticle.

101. The kit of any one of claims 95-100, wherein the melanoma antigen is present within a panel of two or more distinct melanoma antigens.

102. The kit of claim 101, wherein the panel comprises SIVA1, IGF2BP2, AQP4, C15orf48, CTAG1B and PAPOLG.

103. The kit of claim 101 or claim 102, wherein the panel comprises HSPA2, SMAD9, HIST2H2AA3 and S100A8.

104. The kit of any one of claims 101-103, wherein the panel comprises FRS2, BIRC5, EIF3E, CENPH and PAPOLG.

105. The kit of any one of claims 101-104, wherein the panel comprises POLR3B, ELMO2, RFWD2, SQSTM1, HSPD1 and IL17A.

106. The kit of any one of claims 101-105, wherein the panel comprises NOVA2, EOMES, SSB, IGF2BP2, ACTB, MLLT6, SNRPD1, TRAF3IP3, C17orf85, HES1, GSK3A, XRCC5, XRCC6, PPP1R2, C15orf48, PTPRR, MAZ, FLNA, TEX264, SNRNP70, CEP131, SNRPA, CENPV, NRIP1, CCNB1, RALY, CALR, GNAI2 and IL36RN.

107. The kit of any one of claims 100-106, wherein the panel comprises CXXC1, LARP1, EGLN2, RPRM, WHSC1L1 and S100A8.

108. The kit of any one of claims 101-107, wherein the panel comprises SUM02, GRP, SDCBP, AMPH, GPHN, BAG6, BICD2, TMEM98, MUM1, CTSW, NCOA1, MIF, SPA17, FGFR1 and KRT19.

109. The kit of any one of claims 101-108, wherein the panel comprises NCOA1, MIF, SDCB4, MUM1, FGFR1 and KRT19.

110. The kit of any one of claims 101-109, wherein the panel comprises: MIF, NCOA1, FGFR1 and SDCBP.

111. The kit of any one of claims 101-110, wherein the panel comprises: SUMO2, GRP and MIF.

112. The kit of any one of claims 101-111, wherein the panel comprises: GRK6 and GRAMD4.

113. The kit of any one of claims 101-112, wherein the panel comprises: TEX264, CREB3L1, HSPA1B, SPTB, MUC12, ERBB3, CASP10, FOXO1, FRS2, PPP1R12A and CAP2.

114. The kit of any one of claims 101-113, wherein the panel comprises: GNG12, CCDC51, USB1, GRAMD4, RPS6KA1 and BCL7B.

115. The kit of any one of claims 101-114, wherein the panel comprises: EOMES, CREB3L1, FRS2, PLIN2, SIPA1L1, ABCB8, MAPT, XRCC5, XRCC6, UBAP1, TRIP4 and EIF4E2.

116. The kit of any one of claims 101-115, wherein the panel comprises: S100A14, MMP3, SHC1, CSNK2A1, DFFA, S100A8, HDAC1, MSH2, CEACAM5, DHFR and ARRB1.

117. The kit of any one of claims 101-116, wherein the panel comprises: FADD, OGT, HSPB1, CAP2, ATP13A2, SIGIRR, TEX264, HSPA1B, SPTB, PDCD6IP, RAPGEF3, ERBB3, PECAM1, PPL, TONSL, ELMO2, LAMB2, BTRC, SUFU, LGALS3BP, KLKB1, EGFR and TOLLIP.

118. The kit of any one of claims 101-117, wherein the panel comprises MAGED2, PIAS3, MITF, AP2B1, PRKCI, AKT2, BTBD2, UBE2Z, L1CAM, GABARAPL2, LAMC1, RPLP2, AMPH, AP1S1, LEPR, TP53, IL23A, CFB, FGA, IL3, IL4R, TMEM98, KDM4A, UBTF, CASP8, PCDH1, RELT, SPTBN1, RPLP2, KRT7, MUM1, FN1, MAGEB4, CTSW, ATG4D, TPM2 and SPA17.

119. The kit of any one of claims 101-118, wherein the panel comprises IL4R, L1CAM, MITF, PIAS3, AP1S1, ATG4D and RPLP2.

120. The kit of any one of claims 101-119, wherein the panel comprises RELT, CASP8, UBE2Z, IL4R, LAMC1, L1CAM and MITF.

121. The kit of any one of claims 101-120, wherein the panel comprises PIAS3, RPLP2, ATG4D, KRT7, TPM2, GABARAPL2 and MAGEB4.

122. The kit of any one of claims 101-121, wherein the panel comprises MAGED2, PIAS3, MITF, AP2B1 and PRKC1.

123. The kit of any one of claims 95-122, wherein the test sample is selected from the group consisting of plasma, serum, whole blood, urine, sweat, lymph, faeces, cerebrospinal fluid, ascites fluid, pleural effusion, seminal fluid, sputum, nipple aspirate, post-operative seroma, saliva, amniotic fluid, tears and wound drainage fluid.

124. Use of one or more melanoma antigens selected from the following:
ABCB8, ACTB, AKT2, AMPH, AP1S1, AP2B1, AQP4, ARRB1, ATG4D, ATP13A2, BAG6, BCL7B, BICD2, BIRC5, BTBD2, BTRC, C15orf48, C17orf85, CALR, CAP2, CASP10, CASP8, CCDC51, CCNB1, CEACAM5, CENPH, CENPV, CEP131, CFB, CREB3L1, CSNK2A1, CTAG1B, CTSW, CXXC1, DFFA, DHFR, EGFR, EGLN2, EIF4E2, ELMO2, EOMES, ERBB3, FADD, FGA, FGFR1, FLNA, FN1, FOXO1, FRS2, GABARAPL2, GNAI2, GNG12, GPHN, GRAMD4, GRK6, GRP, GSK3A, HDAC1, HES1, HIST2H2AA3, HSPA1B, HSPA2, HSPB1, HSPD1, IGF2BP2, IL3, IL4R, IL17A, IL23A, IL36RN, KDM4A, KLKB1, KRT7, KRT19, L1CAM, LAMB2, LAMC1, LARP1, LEPR, LGALS3BP, MAGEB4, MAGED2, MAPT, MAZ, MIF, MITF, MLLT6, MMP3, MSH2, MUM1, MUC12, NCOA1, NOVA2, NRIP1, OGT, PAPOLG, PCDH1, PDCD6IP, PECAM1, PIAS3, PLIN2, POLR3B, PPL, PPP1R12A, PPP1R2, PRKCI, PTPRR, RALY, RAPGEF3, RELT, RFWD2, RPLP0, RPLP2, RPRM, RPS6KA1, S100A8, S100A14, SDCBP, SHC1, SIGIRR, SIPA1L1, SIVA1, SMAD9, SNRNP70, SNRPA, SNRPD1, SQSTM1, SPA17, SPTB, SPTBN1, SSB, SUFU, SUM02, TEX264, TMEM98, TOLLIP, TONSL, TP53, TPM2, TRAF3IP3, TRIP4, UBAP1, UBE2Z, UBTF, USB1, WHSC1L1, XRCC5 and XRCC6;
in a method for selecting a melanoma patient for treatment with a checkpoint inhibitor wherein the method is performed in accordance with any one of claims 1-31.

125. Use of one or more melanoma antigens selected from the following:
ACTB, AQP4, ARRB1, BCL7B, BIRC5, C15orf48, C17orf85, CALR, CCDC51, CCNB1, CEACAM5, CENPH, CENPV, CEP131, CSNK2A1, CTAG1B, DFFA, DHFR, EIF3E, EOMES, FGA, FGFR1, FLNA, FRS2, GNAI2, GNG12, GPHN, GRAMD4, GRK6, GSK3A, HDAC1, HES1, IGF2BP2, IL36RN, MAZ, MIF, MLLT6, MMP3, MSH2, NOVA2, NRIP1, PAPOLG, PPP1R2, PTPRR, RALY, RPS6KA1, S100A14, S100A8, SHC1, SIVA1, SNRNP70, SNRPA, SNRPD1, SSB, TEX264, TRAF3IP3, USB1, XRCC5 and XRCC6;
in a method for predicting a melanoma patient's responsiveness to treatment with a checkpoint inhibitor wherein the method is performed in accordance with any one of claims 33-40.

126. Use of one or more melanoma antigens selected from the following:
ACTB, AQP4, ARRB1, BCL7B, BIRC5, C15orf48, C17orf85, CALR, CCDC51, CCNB1, CEACAM5, CENPH, CENPV, CEP131, CSNK2A1, CTAG1B, DFFA, DHFR, EIF3E, EOMES, FGA, FGFR1, FLNA, FRS2, GNAI2, GNG12, GPHN, GRAMD4, GRK6, GSK3A, HDAC1, HES1, IGF2BP2, IL36RN, MAZ, MIF, MLLT6, MMP3, MSH2, NOVA2, NRIP1, PAPOLG, PPP1R2, PTPRR, RALY, RPS6KA1, S100A14, S100A8, SHC1, SIVA1, SNRNP70, SNRPA, SNRPD1, SSB, TEX264, TRAF3IP3, USB1, XRCC5 and XRCC6;
in a method for predicting a melanoma patient's survival responsive to treatment with a checkpoint inhibitor wherein the method is performed in accordance with any one of claims 41-48.

127. Use of one or more melanoma antigens selected from the following:
ABCB8, AKT2, AMPH, AP1S1, AP2B1, ARRB1, ATG4D, ATP13A2, BAG6, BICD2, BTBD2, BTRC, CAP2, CASP10, CASP8, CEACAM5, CFB, CREB3L1, CSNK2A1, CTSW, CXXC1, DFFA, DHFR, EGFR, EGLN2, EIF4E2, ELMO2, EOMES, ERBB3, FADD, FGA, FGFR1, FN1, FOXO1, FRS2, GABARAPL2, GPHN, GRP, HDAC1, HIST2H2AA3, HSPA1B, HSPA2, HSPD1, IL17A, IL23A, IL3, IL4R, KDM4A, KLKB1, KRT19, KRT7, L1CAM, LAMB2, LAMC1, LARP1, LEPR, LGALS3BP, MAGEB4, MAGED2, MAPT, MIF, MITF, MMP3, MSH2, MUC12, MUM1, NCOA1, OGT, PCDH1, PDCD6IP, PECAM1, PIAS3, PLIN2, POLR3B, PPL, PPP1R12A, PRKCI, RAPGEF3, RELT, RFWD2, RPLP0, RPLP2, RPRM, S100A14, S100A8, SDCBP, SHC1, SIGIRR, SIPA1L1, SMAD9, SPA17, SPTB, SPTBN1, SQSTM1, SUFU, SUM02, TEX264, TMEM98, TOLLIP, TONSL, TP53, TPM2, TRIP4, UBAP1, UBE2Z, UBTF, WHSC1L1, XRCC5 and XRCC6;
in a method for predicting an immune-related adverse event (irAE) in a melanoma patient treated with a checkpoint inhibitor wherein the method is performed in accordance with any one of claims 49-82.

128. Use of one or more melanoma antigens selected from the following:
RPLP2, CTAG1B, EEF2, CXCL5, DNAJC8, CREB3L1, AKT3, CXCL13, NME1, ANXA4, AKAP13, CDR2L, ATP1B3, DUSP3, SDC1, CPSF1, GRK2, TRA2B, BCR, CSNK2A1, ARRB1, GRK6, CTAG2, MIF, ERBB3, SUFU, BTRC, SIGIRR, SIPA1L1, ACTB, MLLT6, SHC1, CAP2, GPHN, AQP4, NOVA2, SNRPA, NRIP1, UBAP1, TEX264, PLIN2, LAMC1, CENPH, USB1, ABCB8, C15orf48/NMES1 and MAGED1;
in a method for detecting or diagnosing melanoma in a mammalian subject wherein the method is performed in accordance with any one of claims 83-94.
